SEQUENCE LISTING (4 sequences)

SEQ ID NO: 1 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 2 - protein, 176 aa, Homo sapiens
MRIFAVFIFMTYWHLLNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 3 - DNA, 3691 bp, Homo sapiens
ggcgcaacgctgagcagctggcgcgtcccgcgcggccccagttctgcgcagcttcccgaggctccgcaccagccgcgcttctgtccgcctgcagggcattccagaaagatgaggatatttgctgtctttatattcatgacctactggcatttgctgaacgcatttactgtcacggttcccaaggacctatatgtggtagagtatggtagcaatatgacaattgaatgcaaattcccagtagaaaaacaattagacctggctgcactaattgtctattgggaaatggaggataagaacattattcaatttgtgcatggagaggaagacctgaaggttcagcatagtagctacagacagagggcccggctgttgaaggaccagctctccctgggaaatgctgcacttcagatcacagatgtgaaattgcaggatgcaggggtgtaccgctgcatgatcagctatggtggtgccgactacaagcgaattactgtgaaagtcaatgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaagcttttcaatgtgaccagcacactgagaatcaacacaacaactaatgagattttctactgcacttttaggagattagatcctgaggaaaaccatacagctgaattggtcatcccagaactacctctggcacatcctccaaatgaaaggactcacttggtaattctgggagccatcttattatgccttggtgtagcactgacattcatcttccgtttaagaaaagggagaatgatggatgtgaaaaaatgtggcatccaagatacaaactcaaagaagcaaagtgatacacatttggaggagacgtaatccagcattggaacttctgatcttcaagcagggattctcaacctgtggtttaggggttcatcggggctgagcgtgacaagaggaaggaatgggcccgtgggatgcaggcaatgtgggacttaaaaggcccaagcactgaaaatggaacctggcgaaagcagaggaggagaatgaagaaagatggagtcaaacagggagcctggagggagaccttgatactttcaaatgcctgaggggctcatcgacgcctgtgacagggagaaaggatacttctgaacaaggagcctccaagcaaatcatccattgctcatcctaggaagacgggttgagaatccctaatttgagggtcagttcctgcagaagtgccctttgcctccactcaatgcctcaatttgttttctgcatgactgagagtctcagtgttggaacgggacagtatttatgtatgagtttttcctatttattttgagtctgtgaggtcttcttgtcatgtgagtgtggttgtgaatgatttcttttgaagatatattgtagtagatgttacaattttgtcgccaaactaaacttgctgcttaatgatttgctcacatctagtaaaacatggagtatttgtaaggtgcttggtctcctctataactacaagtatacattggaagcataaagatcaaaccgttggttgcataggatgtcacctttatttaacccattaatactctggttgacctaatcttattctcagacctcaagtgtctgtgcagtatctgttccatttaaatatcagctttacaattatgtggtagcctacacacataatctcatttcatcgctgtaaccaccctgttgtgataaccactattattttacccatcgtacagctgaggaagcaaacagattaagtaacttgcccaaaccagtaaatagcagacctcagactgccacccactgtccttttataatacaatttacagctatattttactttaagcaattcttttattcaaaaaccatttattaagtgcccttgcaatatcaatcgctgtgccaggcattgaatctacagatgtgagcaagacaaagtacctgtcctcaaggagctcatagtataatgaggagattaacaagaaaatgtattattacaatttagtccagtgtcatagcataaggatgatgcgaggggaaaacccgagcagtgttgccaagaggaggaaataggccaatgtggtctgggacggttggatatacttaaacatcttaataatcagagtaattttcatttacaaagagaggtcggtacttaaaataaccctgaaaaataacactggaattccttttctagcattatatttattcctgatttgcctttgccatataatctaatgcttgtttatatagtgtctggtattgtttaacagttctgtcttttctatttaaatgccactaaattttaaattcatacctttccatgattcaaaattcaaaagatcccatgggagatggttggaaaatctccacttcatcctccaagccattcaagtttcctttccagaagcaactgctactgcctttcattcatatgttcttctaaagatagtctacatttggaaatgtatgttaaaagcacgtatttttaaaatttttttcctaaatagtaacacattgtatgtctgctgtgtactttgctatttttatttattttagtgtttcttatatagcagatggaatgaatttgaagttcccagggctgaggatccatgccttctttgtttctaagttatctttcccatagcttttcattatctttcatatgatccagtatatgttaaatatgtcctacatatacatttagacaaccaccatttgttaagtatttgctctaggacagagtttggatttgtttatgtttgctcaaaaggagacccatgggctctccagggtgcactgagtcaatctagtcctaaaaagcaatcttattattaactctgtatgacagaatcatgtctggaacttttgttttctgctttctgtcaagtataaacttcactttgatgctgtacttgcaaaatcacattttctttctggaaattccggcagtgtaccttgactgctagctaccctgtgccagaaaagcctcattcgttgtgcttgaacccttgaatgccaccagctgtcatcactacacagccctcctaagaggcttcctggaggtttcgagattcagatgccctgggagatcccagagtttcctttccctcttggccatattctggtgtcaatgacaaggagtaccttggctttgccacatgtcaaggctgaagaaacagtgtctccaacagagctccttgtgttatctgtttgtacatgtgcatttgtacagtaattggtgtgacagtgttctttgtgtgaattacaggcaagaattgtggctgagcaaggcacatagtctactcagtctattcctaagtcctaactcctccttgtggtgttggatttgtaaggcactttatcccttttgtctcatgtttcatcgtaaatggcataggcagagatgatacctaattctgcatttgattgtcactttttgtacctgcattaatttaataaaatattcttatttattttgttacttggtacaccagcatgtccattttcttgtttattttgtgtttaataaaatgttcagtttaacatcccagtggagaaagttaaaaaa

SEQ ID NO: 4 - DNA, 3349 bp, Homo sapiens
ggcgcaacgctgagcagctggcgcgtcccgcgcggccccagttctgcgcagcttcccgaggctccgcaccagccgcgcttctgtccgcctgcagggcattccagaaagatgaggatatttgctgtctttatattcatgacctactggcatttgctgaacgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaagcttttcaatgtgaccagcacactgagaatcaacacaacaactaatgagattttctactgcacttttaggagattagatcctgaggaaaaccatacagctgaattggtcatcccagaactacctctggcacatcctccaaatgaaaggactcacttggtaattctgggagccatcttattatgccttggtgtagcactgacattcatcttccgtttaagaaaagggagaatgatggatgtgaaaaaatgtggcatccaagatacaaactcaaagaagcaaagtgatacacatttggaggagacgtaatccagcattggaacttctgatcttcaagcagggattctcaacctgtggtttaggggttcatcggggctgagcgtgacaagaggaaggaatgggcccgtgggatgcaggcaatgtgggacttaaaaggcccaagcactgaaaatggaacctggcgaaagcagaggaggagaatgaagaaagatggagtcaaacagggagcctggagggagaccttgatactttcaaatgcctgaggggctcatcgacgcctgtgacagggagaaaggatacttctgaacaaggagcctccaagcaaatcatccattgctcatcctaggaagacgggttgagaatccctaatttgagggtcagttcctgcagaagtgccctttgcctccactcaatgcctcaatttgttttctgcatgactgagagtctcagtgttggaacgggacagtatttatgtatgagtttttcctatttattttgagtctgtgaggtcttcttgtcatgtgagtgtggttgtgaatgatttcttttgaagatatattgtagtagatgttacaattttgtcgccaaactaaacttgctgcttaatgatttgctcacatctagtaaaacatggagtatttgtaaggtgcttggtctcctctataactacaagtatacattggaagcataaagatcaaaccgttggttgcataggatgtcacctttatttaacccattaatactctggttgacctaatcttattctcagacctcaagtgtctgtgcagtatctgttccatttaaatatcagctttacaattatgtggtagcctacacacataatctcatttcatcgctgtaaccaccctgttgtgataaccactattattttacccatcgtacagctgaggaagcaaacagattaagtaacttgcccaaaccagtaaatagcagacctcagactgccacccactgtccttttataatacaatttacagctatattttactttaagcaattcttttattcaaaaaccatttattaagtgcccttgcaatatcaatcgctgtgccaggcattgaatctacagatgtgagcaagacaaagtacctgtcctcaaggagctcatagtataatgaggagattaacaagaaaatgtattattacaatttagtccagtgtcatagcataaggatgatgcgaggggaaaacccgagcagtgttgccaagaggaggaaataggccaatgtggtctgggacggttggatatacttaaacatcttaataatcagagtaattttcatttacaaagagaggtcggtacttaaaataaccctgaaaaataacactggaattccttttctagcattatatttattcctgatttgcctttgccatataatctaatgcttgtttatatagtgtctggtattgtttaacagttctgtcttttctatttaaatgccactaaattttaaattcatacctttccatgattcaaaattcaaaagatcccatgggagatggttggaaaatctccacttcatcctccaagccattcaagtttcctttccagaagcaactgctactgcctttcattcatatgttcttctaaagatagtctacatttggaaatgtatgttaaaagcacgtatttttaaaatttttttcctaaatagtaacacattgtatgtctgctgtgtactttgctatttttatttattttagtgtttcttatatagcagatggaatgaatttgaagttcccagggctgaggatccatgccttctttgtttctaagttatctttcccatagcttttcattatctttcatatgatccagtatatgttaaatatgtcctacatatacatttagacaaccaccatttgttaagtatttgctctaggacagagtttggatttgtttatgtttgctcaaaaggagacccatgggctctccagggtgcactgagtcaatctagtcctaaaaagcaatcttattattaactctgtatgacagaatcatgtctggaacttttgttttctgctttctgtcaagtataaacttcactttgatgctgtacttgcaaaatcacattttctttctggaaattccggcagtgtaccttgactgctagctaccctgtgccagaaaagcctcattcgttgtgcttgaacccttgaatgccaccagctgtcatcactacacagccctcctaagaggcttcctggaggtttcgagattcagatgccctgggagatcccagagtttcctttccctcttggccatattctggtgtcaatgacaaggagtaccttggctttgccacatgtcaaggctgaagaaacagtgtctccaacagagctccttgtgttatctgtttgtacatgtgcatttgtacagtaattggtgtgacagtgttctttgtgtgaattacaggcaagaattgtggctgagcaaggcacatagtctactcagtctattcctaagtcctaactcctccttgtggtgttggatttgtaaggcactttatcccttttgtctcatgtttcatcgtaaatggcataggcagagatgatacctaattctgcatttgattgtcactttttgtacctgcattaatttaataaaatattcttatttattttgttacttggtacaccagcatgtccattttcttgtttattttgtgtttaataaaatgttcagtttaacatcccagtggagaaagttaaaaaa

(19) United States
(12) Patent Application Publication
(10) Pub. No.: US 2022/0107320 A1
(43) Pub. Date: Apr. 7, 2022

(54) Assaying Bladder-Associated Samples, Identifying and Treating Bladder-Associated Neoplasia, and Kits for Use Therein

(71) Applicant: IncellDx, Inc., San Carlos, CA (US)

(72) Inventors: Amanda Noel Chargin, San Carlos, CA (US); Pin-I Chen, San Carlos, CA (US); Bruce K. Patterson, San Carlos, CA (US)

(21) Appl. No.: 17/429,817

(22) PCT Filed: Feb. 14, 2020
(86) PCT No.: PCT/US2020/018367

§ 371 Date: Aug. 10, 2021

Related U.S. Application Data
(60) Provisional application No. 62/806,531, filed on Feb. 15, 2019.
(60) Provisional application No. 62/836,526, filed on Apr. 19, 2019.

(51) Int. Cl. G01N 33/574; G01N 1/30; G01N 15/14

(52) U.S. Cl. CPC G01N 33/57407; G01N 1/30; G01N 15/1434; G01N 2015/1006; G01N 2333/70589; G01N 2333/4742; G01N 2333/70596

(57) ABSTRACT

Methods are provided for assaying bladder-associated samples. Aspects of the methods include detecting per cell programmed-death ligand 1 (PD-L1) expression in a bladder-associated sample. In some instances, the methods include detecting whether an immune cell that expresses PD-Ll above a predetermined threshold is present in a bladder-associated sample and/or detecting a PD-L1-aneuploid-to-PD-L1-epithelial ratio of a bladder-associated sample. Aspects of the methods may also include identifying whether a malignant bladder-associated neoplasia is present. Methods are also provided for treating a subject for a malignant bladder-associated neoplasia, wherein aspects of such methods include administering a therapeutic to a subject having an identified malignant bladder-associated neoplasia. In addition, kits that find use in practicing the subject methods are also provided.

### CROSS-REFERENCE TO RELATED APPLICATIONS

Pursuant to 35 U.S.C. § 119 (e), this application claims priority to the filing dates of U.S. Provisional Patent Application Ser. No. 62/806,531 filed Feb. 15, 2019 and 62/836,526 filed Apr. 19, 2019, the disclosures of which applications are herein incorporated by reference.

### INTRODUCTION

Cancer remains one of the leading causes of death globally, with an estimated 12.7 million annual cases around the world affecting both sexes. This number is expected to increase to 21 million by 2030. Currently, there are over 70,000 cases of bladder cancer annually with a male-to-female ratio of 4 to 1 resulting in over 2 cases per 100,000 females and nearly 10 cases per 100,000 males. Bladder cancer is most common in people of Caucasian descent and overall cases appear in subjects with a median age of 60-70 years. Bladder cancer is responsible for about 13,000 deaths annually.

Hematuria is one symptom of bladder cancer. While many cases of hematuria (blood in urine) are due to benign causes, hematuria can also be an indication of serious conditions, including bladder cancer. Visible blood in the urine is referred to as gross hematuria, whereas blood that is only visible under a microscope is referred to as microscopic hematuria or microhematuria. Rates of microhematuria range from 2.4% to 31.1% depending on demographic grouping, with higher rates in males over age 60 years and in men who are current or past smokers. Complicating diagnosis, the threshold to define microhematuria depends on age, gender, frequency of testing, and study group characteristics, such as the presence of risk factors (e.g., past or current smoking). Higher rates of microhematuria are also found in samples that are repeatedly tested.

Asymptomatic microhematuria (AMH) is defined as three or greater red blood cells (RBC) per high powered field (HPF) on a properly collected urinary specimen in the absence of an obvious benign cause. Once benign causes have been ruled out, the presence of AMH generally prompts a urologic evaluation. For the urologic evaluation of AMH, a cystoscopy may be performed, particularly on patients aged 35 years and older. A cystoscopy is generally performed on all patients who present with risk factors for urinary tract malignancies (e.g., irritative voiding symptoms, current or past tobacco use, chemical exposures, etc.) regardless of age. The initial evaluation for AMH generally includes a radiologic evaluation. Multi-phasic computed tomography (CT) urography (without and with intravenous (IV) contrast), including sufficient phases to evaluate the renal parenchyma to rule out a renal mass and an excretory phase to evaluate the urothelium of the upper tracts, is the imaging procedure of choice because it has the highest sensitivity and specificity for imaging the upper tracts. For persistent asymptomatic microhematuria after negative urologic work up, yearly urinalyses are generally conducted. For persistent or recurrent asymptomatic microhematuria after initial negative urologic work-up, repeat evaluation within three to five years should be considered. Overall, microhematuria is associated with malignancy in 3-5% of cases.

The immune system is intimately involved with malignancy, playing a particularly decisive role during disease progression to metastasis. The impact of the immune system on a cancer is not strictly inhibitory as the complex cross talk between immunity and cancer cells also enhances tumor growth. The involvement of the immune system in cancer progression is now generally regarded as a hallmark of cancer. Thus, how the immune system responds to a cancer determines the eventual outcome. Even in cases where a subject's immune system does mount a significant initial response to a cancer, the cancer may still evade the destructive elements of the immune response through various mechanisms including the expression of immune check-point proteins to trigger immune suppression. Further mechanisms resulting in evasion of immune attack include the selection of tumor variants resistant to immune effectors (i.e., “immuno-editing”) and progressive formation of an immune suppressive environment within the tumor.

Immunotherapies seek to rationally redirect a subject's immune system to effectively target the cancer and/or prevent immune evasion.

### SUMMARY

Methods are provided for assaying bladder-associated samples. Aspects of the methods include detecting per cell programmed-death ligand 1 (PD-L1) expression in a bladder-associated sample. In some instances, the methods include detecting whether an immune cell that expresses PD-L1 above a predetermined threshold is present in a bladder-associated sample and/or detecting a PD-L1-aneuploid-to-PD-L1-epithelial ratio of a bladder-associated sample. Aspects of the methods may also include identifying whether a malignant bladder-associated neoplasia is present. Methods are also provided for treating a subject for a malignant bladder-associated neoplasia, wherein aspects of such methods include administering a therapeutic to a subject having an identified malignant bladder-associated neoplasia. In addition, kits that find use in practicing the subject methods are also provided.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIG. 1 shows nucleated cell recovery from urine samples and sample adequacy obtained using various cell preparation reagents.

FIG. 2 shows epithelial cell recovery from urine samples and sample adequacy obtained using various cell preparation reagents.

FIG. 3 provides ROC curves for the DNA index parameter as analyzed from bladder-associated cells prepared with two different cell preparation reagents.

FIG. 4 provides ROC curves for the percent (%) aneuploid epithelial cell parameter as analyzed from bladder-associated cells prepared with two different cell preparation reagents.

FIG. 5 provides ROC curves for the percent (%) single nucleated white blood cells (WBC) parameter as analyzed from bladder-associated cells prepared with two different cell preparation reagents.

FIG. 6 provides ROC curves for the percent (%) PD-L1(+) white blood cells parameter as analyzed from bladder-associated cells prepared with two different cell preparation reagents.

FIG. 7 provides box-and-whisker plots summarizing the statistical findings for percent single nucleated white blood cells (% WBC) of normal and bladder cancer patient samples.

FIG. 8 provides box-and-whisker plots summarizing the statistical findings for percent (%) aneuploid epithelial cells of normal and bladder cancer patient samples.

FIG. 9 provides box-and-whisker plots summarizing the statistical findings for DNA index of normal and bladder cancer patient samples.

FIG. 10 provides box-and-whisker plots summarizing the statistical findings for percent (%) PD-L1(+) WBC of normal and bladder cancer patient samples.

FIG. 11 provides box-and-whisker plots summarizing the statistical findings for percent (%) PD-L1(+) epithelial cells of normal and bladder cancer patient samples.

FIG. 12 provides a flow chart depicting the determination of a PD-L1-aneuploid-to-PD-L1-epithelial ratio (also referred to as “PDL1 Ratio”) using measured and determined features as described below.

FIG. 13 provides a plot of determined PDL1 Ratio and determined epithelial-aneuploid content value (also referred to as “Aneuploid Content”) of normal samples (closed circles) and cancerous samples (open circles) as described below.

FIG. 14 depicts the plot provided in FIG. 13 with a PDL1 Ratio threshold of 0.18 and an Aneuploid Content threshold of 0.09 as described below.

FIGS. 15 to 17 depict results from an experiment detailed in the Experimental section, below.

### DETAILED DESCRIPTION

Methods are provided for assaying bladder-associated samples. Aspects of the methods include detecting per cell programmed-death ligand 1 (PD-L1) expression in a bladder-associated sample. In some instances, the methods include detecting whether an immune cell that expresses PD-L1 above a predetermined threshold is present in a bladder-associated sample and/or detecting a PD-L1-aneuploid-to-PD-L1-epithelial ratio of a bladder-associated sample. Aspects of the methods may also include identifying whether a malignant bladder-associated neoplasia is present. Methods are also provided for treating a subject for a malignant bladder-associated neoplasia, wherein aspects of such methods include administering a therapeutic to a subject having an identified malignant bladder-associated neoplasia. In addition, kits that find use in practicing the subject methods are also provided.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

Methods

As summarized above, methods are provided for assaying bladder-associated samples, including detecting per cell programmed-death ligand 1 (PD-L1) expression in a bladder-associated sample. In some instances, aspects of the methods involve detecting immune cells that expresses PD-L1 in the bladder-associated sample and/or detecting a PD-L1-aneuploid-to-PD-L1-epithelial ratio of a bladder-associated sample. By “bladder-associated sample” is meant a sample that is physically and/or functionally associated with the bladder of a subject. A bladder-associated sample may be used to detect whether an immune cell expressing PD-L1 is present therein and/or whether a malignant bladder-associated neoplasia is present in the subject from which the sample was derived. A bladder-associated sample may be used to detect a PD-L1-aneuploid-to-PD-L1-epithelial ratio of the sample, including e.g., where such a ratio is employed to identify the presence or absence of a malignant bladder-associated neoplasm in the subject from which the sample was derived. In some instances, the ratio may be employed to differentiate between high grade (HG) and low grade (LG) transitional cell carcinoma of the bladder (BC). As described in more detail below, useful bladder-associated samples include but are not limited to e.g., urine, cytology effluent, bladder biopsy samples, samples from the urinary tract or an organ or tissue related thereto, and the like.

Bladder associated samples may be obtained from subjects that do or do not have one or more risk factors for a bladder-related neoplasia. For example, in some instances, a bladder associated sample may be obtained from a healthy subject, e.g., for screening purposes, where such subject may or may not have one or more risk factors for a bladder-related neoplasia. In some instances, a bladder associated sample may be obtained from a subject having one or more symptoms associated with a disorder, such as e.g., a bladder-related neoplasia. Subjects having one or more symptoms associated with bladder-related neoplasia may or may not have one or more risk factors for bladder-related neoplasia.

Risk factors for bladder-related neoplasia, that may or may not be present in a relevant subject, include but are not limited to e.g., smoking, previous urothelial cancer, exposure to carcinogens (e.g., aromatic amines, benzidine, alanine dyes, etc.), urinary stasis (including e.g., diverticulum), chronic bladder and/or urinary tract infection/irritation (including e.g., indwelling catheter (IDC), urinary stones, urinary tract infection (UTI), etc.), and the like. Other common risk factors for urinary tract malignancy, such as in patients with microhematuria, include male gender, age (e.g., >35 years), past or current smoking, occupational or other exposure to chemicals or dyes (benzenes or aromatic amines), analgesic abuse, history of gross hematuria, history of urologic disorder or disease, history of irritative voiding symptoms, history of pelvic irradiation, history of chronic urinary tract infection, history of exposure to known carcinogenic agents or chemotherapy such as alkylating agents, history of chronic indwelling foreign body, and the like.

Symptoms of bladder-associated neoplasia may vary and may include but are not limited to e.g., gross hematuria, microhematuria, pain or burning sensation during urination (irritative voiding symptoms), frequent urination, nocturia or nocturnal polyuria, feeling the need to urinate without being able to pass urine, lower back pain (including asymmetrical pain, e.g., on one side of the body), loss of appetite and weight loss, feeling tired or weak, swelling in the feet, bone pain, and the like.

As summarized above, in some instances, microhematuria may be a symptom of a bladder-associated neoplasm. Accordingly, in some instances, a bladder-associated sample of a method of the present disclosure may be obtained from a subject having microhematuria or having been previously diagnosed with microhematuria. In some instances, a bladder-associated sample is a urine sample, including where such a urine sample is a urine sample with microhematuria. In some instances, a bladder-associated sample of a method of the present disclosure may be a healthy urine sample, i.e., a urine sample obtained from a subject that does not display one or more symptoms associated with a bladder-associated neoplasm. Samples, and the subjects from which samples may be derived, are described in greater detail below.

As summarized above, methods of the present disclosure may relate to the detection of a bladder-associated neoplasm. By “bladder-associated neoplasm”, as used herein, is generally meant any neoplasm associated with the bladder or urinary tract or genitourinary system of a subject. Non-limiting examples of bladder-associated neoplasms include but are not limited to e.g., bladder cancers, kidney cancers, penile cancers, vaginal cancers, testicular cancers, urethral cancers, ureter cancers, renal pelvis cancers, and the like.

In some instances, methods of the present disclosure may identify a subject as having a bladder-associated carcinoma, such as but not limited to e.g., a urothelial carcinoma, noninvasive bladder papillary carcinoma, noninvasive bladder carcinoma in situ (CIS), non-muscle-invasive bladder cancer, muscle-invasive bladder cancer, flat carcinoma, squamous cell carcinoma, small cell carcinoma, and adenocarcinoma. Bladder-associated neoplasms can occur at various positions along the urinary tract, including e.g., at the kidney (e.g., in the renal pelvis), in the upper urinary tract (e.g., the ureter), in the bladder, and in the lower urinary tract (e.g., the urethra). Other bladder-associated neoplasms that may be detected, in some instances, include sarcomas, paragangliomas, melanomas, and lymphomas.

Methods of the present disclosure may involve indirectly detecting the presence of a bladder-associated neoplasm, including e.g., where the detection of an immune cell expressing PD-L1 in a bladder-associated sample indirectly indicates the presence of a bladder-associated neoplasm and/or the presence of a malignant bladder neoplasm in the subject from which the same was derived. In some instances, methods of the present disclosure may further involve direct detection of neoplastic cells. In some instances, methods of the present disclosure may involve detecting the presence of a bladder-associated neoplasm based on a parameter derived from measured features of the sample, including but not limited to e.g., a PD-L1-aneuploid-to-PD-L1-epithelial ratio, an epithelial-aneuploid content value, combinations thereof, and the like.

As summarized above, embodiments are directed to methods of detecting per cell programmed-death ligand 1 (PD-L1) expression and detecting a cell that expresses PD-L1 above a predetermined threshold. Cells in which PD-L1 expression may be detected in the subject methods include, in various instances, immune cells, and in some instances non-immune cells, such as e.g., epithelial cells, neoplastic cells, and the like. In some embodiments, PD-L1 detection and/or quantification may be limited only to immune cells. In some embodiments, PD-L1 detection and/or quantification may be limited to only epithelial cells and/or aneuploid cells. Accordingly, in some instances a cell e.g., an immune cell, an epithelial cell, an aneuploid cell, etc., detected in the subject methods may have a per cell expression level of PD-L1 protein that exceeds a predetermined threshold.

Various types of immune cells may be assayed and/or detected. As used herein, the term “immune cells” generally includes white blood cells (leukocytes) which are derived from hematopoietic stem cells (HSC) produced in the bone marrow. “Immune cells” includes, e.g., lymphocytes (T cells, B cells, natural killer (NK) cells) and myeloid-derived cells (neutrophil, eosinophil, basophil, monocyte, macrophage, dendritic cells). “T cell” includes all types of immune cells expressing CD3 including T-helper cells (CD4⁺ cells), cytotoxic T-cells (CD8⁺ cells), T-regulatory cells (Treg) and gamma-delta T cells. A “cytotoxic cell” includes CD8⁺ T cells, natural-killer (NK) cells, and neutrophils, which cells are capable of mediating cytotoxicity responses.

In some instances, immune cells may be defined based on expression (including presence or absence) of one or more markers. For example, in some instances, an immune cell may be a CD45 expressing (i.e., CD45(+)) immune cell. Useful immune cell markers include but are not limited to e.g., CD114, CD117, CD11a, CD11b, CD14, CD15, CD16, CD182, CD19, CD20, CD22, CD24, CD25, CD3, CD30, CD31, CD34, CD38, CD4, CD45, CD56, CD61, CD8, CD91, Foxp3, and the like. Accordingly, in some instances, a detected immune cell may be an immune cell identified based on the presence and/or absence of one or more of CD114, CD117, CD11a, CD11b, CD14, CD15, CD16, CD182, CD19, CD20, CD22, CD24, CD25, CD3, CD30, CD31, CD34, CD38, CD4, CD45, CD56, CD61, CD8, CD91, and Foxp3.

By “per cell expression level of PD-L1” or “per cell expression of PD-L1”, as used herein, is meant the quantity of PD-L1 molecules present on the surface of a cell. Methods of the present disclosure include cytometrically assaying a cellular sample to quantify the per cell expression of PD-L1 and subsequently detecting one or more cells in the sample that have a per cell expression level of PD-L1 protein that exceeds the predetermined threshold. Various means of cytometrically assaying a cellular sample, described in more detail below, may be employed in the subject methods.

As described in more detail below, the level of PD-L1 expression in immune cells assayed in the methods of the present disclosure will vary. For example, in some instances, an immune cell expressing PD-L1 may express PD-L1 above a predetermined threshold where the threshold is 100 or more PD-L1 molecules per cell. In some instances, an immune cell expressing PD-L1 may express PD-L1 above a predetermined threshold where the threshold is 500 or more PD-L1 molecules per cell. In some instances, an immune cell expressing PD-L1 may express PD-L1 above a predetermined threshold where the threshold is 1000 or more PD-L1 molecules per cell.

Accordingly, in some instances, a bladder-associated sample may be assayed for the percentage of cells that are PD-L1(+) immune cells (i.e., immune cells that express PD-L1 above a predetermined threshold). Such assaying may involve contacting the bladder-associated sample with at least one labeled binding member specific for immune cells and a labeled binding member specific for PD-L1, and cytometrically detecting cells to which the labeled binding members are bound. In some instances, the at least one labeled binding member specific for immune cells may include an anti-CD45 antibody. A population of cells, in some instances referred to as events in a cytometric assay, may then be quantified based on the labeled binding members and the percentage of cells of the population that are PD-L1(+) immune cells (based on labeling with the labeled binding members) may be determined.

The percentage of cells that are PD-L1(+) immune cells in a population of cells of an assayed bladder-associated sample may vary and may range from 1% or less to 90% or more, including but not limited to e.g., less than 1%, less than 2%, less than 3%, less than 4%, less than 5%, less than 6%, less than 7%, less than 8%, less than 9%, less than 10%, less than 15%, less than 20% less than 25%, less than 30%, less than 40%, less than 45%, less than 50%, 1% or more, 2% or more, 3% or more, 4%, or more, 5% or more, 6% or more, 7% or more, 8% or more, 9% or more, 10% or more, 15% or more, 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 95% or more, etc.

In some instances, a predetermined threshold may be employed where a percentage of cells that are PD-L1(+) immune cells in an assayed population that is above the predetermined threshold, alone or in combination with other assayed parameters, indicates the presence of a bladder-associated neoplasm and/or a malignant bladder associated neoplasm in the subject from which the sample was derived. Such predetermined thresholds will vary and may include e.g., 1% or more, 2% or more, 3% or more, 4%, or more, 5% or more, 6% or more, 7% or more, 8% or more, 9% or more, 10% or more, 15% or more, 20% or more, 25% or more, 30% or more, 35% or more, 40% or more, 45% or more, 50% or more, 55% or more, 60% or more, 65% or more, 70% or more, 75% or more, 80% or more, etc.

Correspondingly, in some instances, a predetermined threshold may be employed where a percentage of cells that are PD-L1(+) immune cells in an assayed population that is below the predetermined threshold, alone or in combination with other assayed parameters, indicates the absence of a bladder-associated neoplasm and/or a malignant bladder associated neoplasm in the subject from which the sample was derived. Such predetermined thresholds will vary and may include e.g., less than 1%, less than 2%, less than 3%, less than 4%, less than 5%, less than 6%, less than 7%, less than 8%, less than 9%, less than 10%, less than 15%, less than 20% less than 25%, less than 30%, less than 40%, less than 45%, less than 50%, etc.

Methods of the present disclosure may or may not be limited to detecting PD-L1 expressing immune cells. For example, in some instances, methods may alternatively, or further, include cytometrically assaying a labeled cell suspension of the bladder-associated sample to quantify other or additional sample and/or cell parameters, e.g., in place of or in addition to detecting immune cells expressing PD-L1.

Non-limiting examples of sample and/or cell parameters that may be assayed and obtained include the percentage of single cells that are nucleated, the percentage of single cells that are nucleated epithelial cells, the percentage of cells that are immune cells, per cell DNA index, mean sample DNA index, epithelial cell aneuploidy, the percentage of cells that are PD-L1 positive epithelial cells, the percentage of cells that are aneuploid epithelial cells, the percentage of cells that are PD-L1 positive aneuploid epithelial cells, mean fluorescence of PD-L1 positive epithelial cells, mean fluorescence of PD-L1 negative epithelial cells, mean fluorescence of PD-L1 positive aneuploid cells, mean fluorescence of PD-L1 negative aneuploid cells, mean fluorescence of PD-L1 positive aneuploid epithelial cells, mean fluorescence of PD-L1 negative aneuploid epithelial cells, and combinations thereof.

In some instances, assayed parameters may be combined into a composite cytometric parameter (also referred to as a “derived feature”). For example, a composite cytometric parameter may represent a combination or two or more measured parameters, including where the two or more measured parameters are combined according to an appropriate mathematical or statistical operator (e.g., sum, product, division, subtraction, etc.). In some instances, features may be derived for a sample where such derived features represent the composite of two or more measured sample features. In some instances, an individual parameter or the composite cytometric parameter may be employed alone or in combination with another parameter, e.g., together with PD-L1-expressing immune cell data, to identify a condition in the subject from which the sample was derived. In some instances, the condition is the presence of a bladder-associated neoplasia and/or the presence of a malignant bladder-associated neoplasia.

Useful measured features that may be employed to generate one or more derived features include but are not limited to e.g.: Perct_Single nucleated epithelial cells (corresponding to the percent of single cells in the sample that are nucleated and labeled with a labeled binding member specific for epithelial cells), Perct_PD-L1+Epithelial cells (corresponding to the percent of cells labeled with a labeled binding member specific for epithelial cells that are also labeled with a labeled binding member specific for PD-L1), Perct_Single nucleated WBC (corresponding to the percent of single cells in the sample that are nucleated and labeled with a labeled binding member specific for white blood cells), Perct_PD-L1+white blood cells (corresponding to the percent of cells labeled with a labeled binding member specific for WBCs that are also labeled with a labeled binding member specific for PD-L1), Perct_Aneuploid Epithelial cells (corresponding to the percent of cells labeled with a labeled binding member specific for epithelial cells that are also aneuploid), Perct_PD-L1+Aneuploid epithelial cells (corresponding to the percent of aneuploid cells that are also labeled with a labeled binding member specific for PD-L1), DNA Index (corresponding to the ratio of the amount of DNA labeling reagent fluorescence per cell of the sample to the amount expected of a normal diploid cell), PD-L1+Epithelial cells (Mean FI) (corresponding to the mean fluorescence attributable to cells labeled with a labeled binding member specific for epithelial cells and the labeled binding member specific for PD-L1), PD-L1 neg Epithelial cells (Mean FI) (corresponding to the mean fluorescence attributable to cells labeled with a labeled binding member specific for epithelial cells and not with the labeled binding member specific for PD-L1), PD-L1+Aneuploid cells (Mean FI) (corresponding to the mean fluorescence attributable to aneuploid cells labeled with the labeled binding member specific for PD-L1), PD-L1neg Aneuploid cells (Mean FI) (corresponding to the mean fluorescence attributable to aneuploid cells not with the labeled binding member specific for PD-L1), PD-L1+WBC cells (Mean FI) (corresponding to the mean fluorescence attributable to cells labeled with a labeled binding member specific for WBCs and labeled with the labeled binding member specific for PD-L1), and PD-L1neg WBC cells (Mean FI) (corresponding to the mean fluorescence attributable to cells labeled with a labeled binding member specific for WBCs and not with the labeled binding member specific for PD-L1).

Useful derived features, that may be produced from the non-limiting examples of measured features listed above, include but are not limited to e.g., derived index features, derived sample content features, and a derived PD-L1 Ratio parameter.

Useful derived index features include but are not limited to e.g., PDL1 index Epithelial Cells (corresponding to [PD-L1+Epithelial cells (Mean fluorescence)] divided by [PD-L1 negative Epithelial cells (Mean fluorescence)]), PDL1 index Aneuploid Cells (corresponding to [PD-L1+Aneuploid cells (Mean fluorescence)] divided by [PD-L1 negative Aneuploid cells (Mean fluorescence)]), and PDL1 index WBC Cells (corresponding to [PD-L1+WBC cells (Mean fluorescence)] divided by [PD-L1neg WBC cells (Mean FI)]).

Useful derived sample content features include but are not limited to e.g., PDL1 content Epithelial Cells (corresponding to the product of (Perct_Single nucleated epithelial cells), (Perct_PD-L1+Epithelial cells) and (PDL1 index Epithelial Cells)), PDL1 content WBC Cells (corresponding to the product of (Perct_Single nucleated WBC), (Perct_PD-L1+white blood cells) and (PDL1 index WBC Cells)), PDL1 content Aneuploid Cells (corresponding to the product of (Perct_Aneuploid Epithelial cells), (Perct_PD-L1+Aneuploid epithelial cells), and (PDL1 index Aneuploid Cells)), Aneuploid Content (corresponding to the product of (DNA Index) and (Perct_Aneuploid Epithelial cells)), Aneuploid Content PDL1 positive cells (corresponding to the product of (DNA Index), (Perct_Aneuploid Epithelial cells), and (Perct_PD-L1+Aneuploid epithelial cells)), and Aneuploid PDL1 content (corresponding to the product of (Perct_Aneuploid Epithelial cells), (Perct_PD-L1+Aneuploid epithelial cells), and (PDL1 index Aneuploid Cells)).

Useful derived features also include the PD-L1 Ratio parameter, which corresponds to the ratio of PDL1 content Aneuploid Cells to PDL1 content Epithelial Cells (also referred to herein as the “PD-L1-aneuploid-to-PD-L1-epithelial ratio”).

Accordingly, in some instances, a bladder-associated sample may be assayed for the percentage of cells that are immune cells. Such assaying may involve contacting the bladder-associated sample with at least one labeled binding member specific for immune cells and cytometrically detecting cells to which the labeled binding member specific for immune cells is bound. In some instances, the at least one labeled binding member specific for immune cells may include an anti-CD45 antibody. A population of cells, in some instances referred to as events in a cytometric assay, may then be quantified based on the labeled binding member specific for immune cells and the percentage of cells of the population that are immune cells (based on labeling with the labeled binding member) may be determined.

The percentage of cells that are immune cells in a population of cells of an assayed bladder-associated sample may vary and may range from 5% or less to 90% or more, including but not limited to e.g., less than 5%, less than 10%, less than 15%, less than 20% less than 25%, 5% or more, 10% or more, 15% or more, 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, etc.

In some instances, a predetermined threshold may be employed where a percentage of cells that are immune cells in an assayed population that is above the predetermined threshold, alone or in combination with other assayed parameters, indicates the presence of a bladder-associated neoplasm and/or a malignant bladder associated neoplasm in the subject from which the sample was derived. Such predetermined thresholds will vary and may include e.g., 5% or more, 10% or more, 15% or more, 20% or more, 25% or more, 30% or more, 35% or more, 40% or more, 45% or more, 50% or more, 55% or more, 60% or more, 65% or more, 70% or more, 75% or more, etc. For example, in some instances, a further cytometric parameter may be determined that includes the percentage of cells that are immune cells and a predetermined threshold may be employed where the immune cell percentage of 5% or greater.

Correspondingly, in some instances, a predetermined threshold may be employed where a percentage of cells that are immune cells in an assayed population that is below the predetermined threshold, alone or in combination with other assayed parameters, indicates the absence of a bladder-associated neoplasm and/or a malignant bladder associated neoplasm in the subject from which the sample was derived. Such predetermined thresholds will vary and may include e.g., less than 5%, less than 10%, less than 15%, etc.

In some instances, a bladder-associated sample may be assayed for per cell DNA index. Such assaying may involve contacting the bladder-associated sample with a DNA labeling reagent and cytometrically detecting cells labeled with the DNA labeling reagent and/or quantifying the per cell amount of DNA present in the cell based on the DNA labeling reagent. In some instances, the amount of DNA present in a cell may be referred to as the DNA index of the cell, which may be expressed relative to the amount of DNA present in a normal (i.e., diploid) cell.

The DNA index of a cell in an assayed bladder-associated sample may vary and may range from 1.0 to 1.8 or more, including but not limited to e.g., 1.0, 1.1, 1.2, 1.3, 1.4, 1.5, 1.6, 1.7, 1.8, 1.9, 1.1 or greater, 1.2 or greater, 1.3 or greater, 1.4 or greater, 1.5 or greater, 1.6 or greater, less than 1.2, less than 1.1, etc.

In some instances, a predetermined threshold may be employed where a DNA index of a cell (or a number of cells) in an assayed population that is above the predetermined threshold, alone or in combination with other assayed parameters, indicates the presence of a bladder-associated neoplasm and/or a malignant bladder associated neoplasm in the subject from which the sample was derived. Such predetermined thresholds will vary and may include e.g., 1.1 or greater, 1.2 or greater, 1.3 or greater, 1.4 or greater, etc. For example, in some instances, a further cytometric parameter may be determined that includes per cell DNA index and a predetermined threshold may be employed that includes a per cell DNA index of 1.1 or greater.

Correspondingly, in some instances, a predetermined threshold may be employed where a DNA index of a cell (or a number of cells) in an assayed population that is below the predetermined threshold, alone or in combination with other assayed parameters, indicates the absence of a bladder-associated neoplasm and/or a malignant bladder associated neoplasm in the subject from which the sample was derived. Such predetermined thresholds will vary and may include e.g., less than 1.2, less than 1.1, etc.

In some instances, a bladder-associated sample may be assayed for epithelial cell aneuploidy. Such assaying may involve contacting the bladder-associated sample with at least one labeled binding member specific for epithelial cells and a DNA labeling reagent, and cytometrically detecting cells to which the labeled binding member specific for epithelial cells is bound and quantifying DNA content based on the DNA labeling reagent in the detected epithelial cells. In some instances, the at least one labeled binding member specific for epithelial cells may include an anti-pan-cytokeratin antibody. A population of cells, in some instances referred to as events in a cytometric assay, may then be quantified based on the labeled binding member specific for epithelial cells and the DNA labeling reagent to determine a percentage of cells of the population that are epithelial cells (based on labeling with the labeled binding member) and are aneuploid.

Epithelial cell aneuploidy of an assayed bladder-associated sample may vary and may range from 1% or less to 70% or more, including but not limited to e.g., less than 1%, less than 2%, less than 3%, less than 4%, less than 5%, less than 10%, less than 15%, less than 20% less than 25%, 1% or more, 2% or more, 3% or more, 4% or more, 5% or more, 10% or more, 15% or more, 20% or more, 25% or more, 30% or more, 35% or more, 40% or more, 45% or more, 50% or more, 55% or more, 60% or more, 65% or more, 70% or more, etc.

In some instances, a predetermined threshold may be employed where epithelial cell aneuploidy in an assayed population that is above the predetermined threshold, alone or in combination with other assayed parameters, indicates the presence of a bladder-associated neoplasm and/or a malignant bladder associated neoplasm in the subject from which the sample was derived. Such predetermined thresholds will vary and may include e.g., 1% or more, 2% or more, 3% or more, 4% or more, 5%, or more, 10% or more, 15% or more, 20% or more, 25% or more, 30% or more, etc. For example, in some instances, a further cytometric parameter may be determined that includes epithelial cell aneuploidy and a predetermined threshold may be employed that includes an epithelial cell aneuploidy of 5% or greater.

Correspondingly, in some instances, a predetermined threshold may be employed where epithelial cell aneuploidy in an assayed population that is below the predetermined threshold, alone or in combination with other assayed parameters, indicates the absence of a bladder-associated neoplasm and/or a malignant bladder associated neoplasm in the subject from which the sample was derived. Such predetermined thresholds will vary and may include e.g., less than 1%, less than 2%, less than 3%, less than 4%, less than 5%, less than 10%, etc.

In some instances, a bladder-associated sample may be assayed for the percentage of cells that are PD-L1(+) epithelial cells. Such assaying may involve contacting the bladder-associated sample with at least one labeled binding member specific for epithelial cells and at least one labeled binding member specific for PD-L1, and cytometrically detecting cells to which the labeled binding members are bound. A population of cells, in some instances referred to as events in a cytometric assay, may then be quantified based on the labeled binding members and the percentage of cells of the population that are PD-L1(+) epithelial cells (based on labeling with the labeled binding members) may be determined.

The percentage of cells that are PD-L1(+) epithelial cells in a population of cells of an assayed bladder-associated sample may vary and may range from 5% or less to 90% or more, including but not limited to e.g., less than 5%, less than 10%, less than 15%, less than 20% less than 25%, 5% or more, 10% or more, 15% or more, 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, etc.

In some instances, a predetermined threshold may be employed where a percentage of cells that are PD-L1(+) epithelial cells in an assayed population that is above the predetermined threshold, alone or in combination with other assayed parameters, indicates the presence of a bladder-associated neoplasm and/or a malignant bladder associated neoplasm in the subject from which the sample was derived. Such predetermined thresholds will vary and may include e.g., 15% or more, 20% or more, 25% or more, 30% or more, 35% or more, 40% or more, 45% or more, 50% or more, 55% or more, 60% or more, 65% or more, 70% or more, 75% or more, etc.

Correspondingly, in some instances, a predetermined threshold may be employed where a percentage of cells that are PD-L1(+) epithelial cells in an assayed population that is below the predetermined threshold, alone or in combination with other assayed parameters, indicates the absence of a bladder-associated neoplasm and/or a malignant bladder associated neoplasm in the subject from which the sample was derived. Such predetermined thresholds will vary and may include e.g., less than 5%, less than 10%, less than 15%, etc.

Methods of the present disclosure may or may not also include identifying one or more neoplastic cells present in a sample. For example, in some instances, methods of the present disclosure may not directly identify neoplastic cells. In some instances, methods may include directly identifying neoplastic cells.

A cell may by identified as a neoplastic cell using various criteria. For example, in some instances, a cell may be identified as neoplastic, or potentially neoplastic, based on observation of the cellular and/or subcellular morphology of the cell alone or in the context of tissue surrounding the cell. In some instances, a cell behavior may be an indication that a cell is neoplastic, such as but not limited to e.g., proliferation, migration, etc. In some instances, markers, such as but not limited to e.g., proliferation markers, cell surface markers, cancer markers, etc., may be employed to identify a cell as neoplastic. In some instances, assaying gene expression, e.g., expression of a cancer marker, expression of a mutated gene, overexpression, underexpression, etc., may indicate whether a cell is neoplastic.

The methods described herein involve the detection and/or quantification of cell surface proteins, such as e.g., proteinaceous cell surface markers. As used herein, the term “cell surface protein” refers to proteinaceous components of the cell that are at least exposed, partially or completely, on the outer surface of the plasma membrane of cell and thus may be accessed (e.g., by a specific binding member for the cell surface protein) without modulating cell permeability, e.g., without the use of one or more permeabilizing reagents. Cell surface proteins are associated with the surface of the cell upon which such proteins are expressed and may or may not have a transmembrane and/or cytoplasmic domain. Cell surface proteins will include at least a portion of the protein that is outside of the cell, which may be referred to as an extracellular portion. In some instances, cell surface proteins include components of the cell that have a portion exposed on the outer surface of the cell membrane but also contain an intracellular portion and/or a transmembrane portion. The portion of the protein that is outside of the cell may have one or more epitopes to which a specific binding member may bind, such that the protein may display one or more epitopes.

As described in more detail below, the present disclosure also includes methods of treating a subject for bladder-associated neoplasia, including e.g., a malignant bladder-associated neoplasia. Such methods may involve assaying a subject of the bladder-associated neoplasia and/or malignant bladder-associated neoplasia, and when detected, treating the subject. Methods described herein for identifying a subject as having a bladder-associated neoplasia and/or a malignant bladder-associated neoplasia may be employed. Useful treatments, as also described further below, include administering to the subject an anti-PD-1/PD-L1 immunotherapy.

PD-L1 Expressing Cells

As summarized above, the present disclosure provides methods that involve quantifying PD-L1 expression, e.g., on a per cell basis and/or over a population of cells. The subject methods may involve detecting cells expressing PD-L1 above a predetermined threshold and/or the percent of cells of a population that express PD-L1 above the predetermined threshold. PD-L1 expression may be quantified on a per cell basis based on the level of PD-L1 protein expression or the level of PD-L1 encoding transcript (i.e., mRNA) expression. In some instances, the subject method quantifies only per cell PD-L1 protein expression and does not quantify per cell PD-L1 transcript expression. In some instances, a combined method of quantifying both PD-L1 protein levels and PD-L1 transcription levels may be employed. In some instances, the subject method quantifies only per cell PD-L1 transcript expression and does not quantify per cell PD-L1 protein expression.

As described in more detail below, embodiments of the instant methods may include cytometrically assaying a cell suspension to detect a cell expressing PD-L1 above a predetermined threshold. As used herein, the term “cytometrically assaying” describes the measuring of cellular parameters on a cell-by-cell basis where such measuring allows for the detection of individual cells that have, or the counting of a cell population that shares, a certain cellular parameter or set of parameters. One such parameter that is cytologically assayed in the subject methods is per cell expression of PD-L1.

PD-L1 (also known as CD274) binds programmed cell death protein 1 (PD-1), a protein encoded by the PDCD1 gene that is a cell surface receptor expressed on T-cells. PD-1 functions as an immune checkpoint by preventing the activation of T-cells, which reduces autoimmunity and promotes self-tolerance. PD-L1 has been found to be expressed on a number of different cancer cell types. The presence of PD-L1 on a cancer cell inhibits T cell activation, contributing to cancer cell immune evasion. A number of cancer therapies are directed to preventing cancer cell immune evasion by inhibiting the PD-1/PD-L1 interaction. The present disclosure includes detecting cells, including e.g., immune cells, epithelial cells, aneuploid cells, etc., expressing PD-L1 above a predetermined threshold. Samples containing immune cells having a per cell PD-L1 expression level above a predetermined threshold may be indicative of the presence of malignant neoplasia. Samples containing cells having a PD-L1-aneuploid-to-PD-L1-epithelial ratio, as determined in part by quantifying per cell PD-L1 expression, may be indicative of the presence of malignant neoplasia.

Cells detected in the methods of the present disclosure will have a level of PD-L1 expression above a predetermined threshold. As such, the methods of the instant disclosure may include cytometrically quantifying per cell expression levels of PD-L1 to identify cells expressing PD-L1 protein and/or PD-L1 transcript above a predetermined threshold. Such cells having a level of PD-L1 expression above a predetermined threshold may be referred to as PD-L1 positive cells and cells having a level of PD-L1 expression below the predetermined threshold may be referred to as PD-L1 negative cells.

Predetermined thresholds for PD-L1 expression useful in the instant disclosure will vary depending on various factors including e.g., the cell type assayed, other measured cellular parameters, and whether PD-L1 protein or transcript are detected. As described in more detail below, PD-L1 expression is determined cytometrically where PD-L1 protein expression may be determined by a variety of protocols including, but not limited to, contacting the cell with a labeled specific binding member that binds PD-L1 protein on the surface of the cell. In some instances, PD-L1 expression is determined cytometrically where PD-L1 transcript expression may be determined by a variety of protocols including, but not limited to, contacting the cell with a labeled specific binding member that binds PD-L1 transcripts within the cell.

In some instances, quantifying per cell PD-L1 expression may include calibrating PD-L1 fluorescence of PD-L1 specific binding partner labeled cells to a reference standard. Depending on the context, a reference standard may be cytometrically assayed in parallel, in series or simultaneously with the assayed cells. For example, in some instances, a reference standard may be cytometrically assayed to calibrate the assay for quantification and then the label cell suspension sample may be assayed using the calibrated cytometric assay. In some instances, a reference standard may be added to (i.e., spiked into) the label cell suspension sample and the calibration based on the reference standard for quantifying the per cell expression of the labeled cells may be performed during cytometric analysis of cells. In some instances, calibration with a reference standard may be performed between or during each run of a labeled cell sample. In some instances, calibration with a reference standard may be performed between or during each batch of runs.

Any convenient reference standard for calibrating labeled cell fluorescence to per cell marker expression may be employed in the herein described assays including but not limited to e.g., standardized microspheres (i.e., beads), standardized control cells, standardized fluorescent particles, and the like. In some instances, spectrally equivalent microsphere standards, such as e.g., Molecules of Equivalent Soluble Fluorochrome (MESF) beads or Mean Equivalent Fluorochrome (MEFL) beads, may be used. Microsphere standards useful in quantitative cytometry will vary any will generally include microspheres labeled with a known amount of fluorophore bound per microsphere or microspheres will a known valency for binding fluorophore labeled molecules. Microsphere standards for quantitative cytometry simulate fluorescent dye attachment to the cell membrane of target cells and allow for calibration of cytometric assays, including e.g., flow cytometric assays or cell cytometric assays, for quantification.

For example, in some instances, microsphere standards for quantitative cytometry will include two or more populations, including e.g., 2 populations, 3 populations, 4 populations, 5 populations, 6 populations, etc., of microspheres labeled with different amounts of a fluorophore. The fluorophore chosen will generally be the same as or equivalent to or comparable with the fluorophore used in one or more of the labeled specific binding members of the described methods. Useful fluorophores in microsphere standards include but are not limited to e.g., Alexa Fluor 488, Alexa Fluor 647, FITC, PE, Cy5, APC, etc. In some instances, two or more microsphere standards having different fluorophores may be mixed, e.g., where quantification of two or more differently labeled specific binding members are used in a subject method. In some instances, microsphere standards having different fluorophores are not mixed and different populations of microspheres having different amounts of a single type of fluorophore bound may be employed.

Microsphere standards may be directly conjugated to the fluorescent label or, in some instances, fluorescently labeled antibody may be bound to the microsphere standard. In some instances, a microsphere standard may be non-fluorescent but “label-able”. Label-able microsphere standards will generally have a known antibody binding capacity allowing for staining of the microsphere with a known amount of a user's antibody, including e.g., the same antibody used as a specific binding member in a herein described method. Label-able microsphere standards may, in some instances, be employed in conjunction with a pre-labeled microsphere standard allowing for determination of the fluorophore to protein (FTP) ratio of the particular labeled specific binding member employed in the method and/or further calibration. Various different microsphere standards, including e.g., fluorescently labeled microsphere standards and label-able microsphere standards, for quantitative cytometry that may find use in the herein described methods include but are not limited to e.g., those commercially available from Bangs Laboratories, Inc. (Fishers, Ind.), BD Biosciences (San Jose, Calif.), and the like.

In some embodiments, the fluorescence of a labeled specific binding member and/or cells labeled with such may be calibrated to microsphere standards (e.g., by assessing the fluorescence of two or more populations of microspheres labeled with different amounts of a fluorophore) to establish a standard curve. Following or during the establishment of a standard curve a labeled cell suspension sample may be assayed and per cell PD-L1 expression may be determined. Quantified per cell PD-L1 expression levels may be compared to a predetermined threshold, including e.g., a threshold established based on the number of molecules of PD-L1 protein expressed per cell, a threshold established based on background fluorescence, a threshold established based on background expression (including e.g., per cell expression) of PD-L1, and the like.

In some instances, a predetermined threshold for per cell PD-L1 expression may be expressed as a number of molecules of the PD-L1 protein per cell, including but not limited to e.g., a threshold of 10 molecules per cell, a threshold of 20 molecules per cell, a threshold of 30 molecules per cell, a threshold of 40 molecules per cell, a threshold of 50 molecules per cell, a threshold of 60 molecules per cell, a threshold of 70 molecules per cell, a threshold of 80 molecules per cell, a threshold of 90 molecules per cell, a threshold of 100 molecules per cell, a threshold of 200 molecules per cell, a threshold of 300 molecules per cell, a threshold of 400 molecules per cell, a threshold of 500 molecules per cell, a threshold of 600 molecules per cell, a threshold of 700 molecules per cell, a threshold of 800 molecules per cell, a threshold of 900 molecules per cell, a threshold of 1000 molecules per cell, a threshold of 1100 molecules per cell, a threshold of 1200 molecules per cell, a threshold of 1300 molecules per cell, a threshold of 1400 molecules per cell, a threshold of 1500 molecules per cell, a threshold of 1600 molecules per cell, a threshold of 1700 molecules per cell, a threshold of 1800 molecules per cell, a threshold of 1900 molecules per cell, a threshold of 2000 molecules per cell, etc.

Accordingly, in some instances, a cell is detected as expressing PD-L1 above a predetermined threshold if the cell is identified as having a per cell number of PD-L1 protein molecules expressed on the surface of the cell that is above one or more of the predetermined thresholds listed above.

In some instances, the methods described herein detect a single cell having a level of PD-L1 expression above a predetermined threshold. In some instances, the presence of a single detected cell having a level of PD-L1 expression above a predetermined threshold is considered significant. In some instances, the methods described herein may include a threshold of cells having a level of PD-L1 expression above a predetermined threshold for the detected cells to be considered significant (i.e., a minimum size for the population of cells having a level of PD-L1 expression above a predetermined threshold to be considered significant). Depending on the context, the size of the detected population of cells expressing PD-L1 above the threshold will vary and may range from one cell to millions of cells, including but not limited to e.g., one cell, one cell or more, 10 cells or more, 100 cells or more, 1,000 cells or more, 10,000 cells or more, 100,000 cells or more.

In some instances, the size of the detected population of cells expressing PD-L1 above the predetermined threshold may be expressed in relative terms. For example, the size of the population may be expressed as a percentage of all the cells in the sample, a percentage of all the cells analyzed, a percentage of all of the cells of a particular type within the sample, a percentage of all of the cells of a particular type that were analyzed, etc. In some instances, the size of the detected population may exceed 0.01% or more of the cells in the cell suspension sample, including but not limited to e.g., 0.1% or more, 1% or more, 10% or more, etc.

In some instances, in order to classify a cell as PD-L1 expressing the size of the population of cells detected as expressing PD-L1 above a predetermined threshold will exceed a predetermined threshold. As described above, the threshold for the size of the detected population will vary based on a number of factors and in some instances may be one cell. In some instances, the threshold for the size of the detected population the population must exceed more than one cell including two cells or more including but not limited to e.g., 0.01% or more of the cells in the sample or a subset of the cells of the sample, 0.1% or more of the cells in the sample or a subset of the cells of the sample, 1% or more of the cells of the sample or a subset of the cells of the sample, and the like.

Useful thresholds also include thresholds for derived features, where such derived features represent a composite of measured features combined according to a mathematical or statistical combination of measured features as described above. For example, in some instances, a method of the present disclosure may include a threshold for a PD-L1-aneuploid-to-PD-L1-epithelial ratio of a sample (i.e., also referred to as a “PD-L1 Ratio”), where such corresponds to the ratio of PD-L1 Content Aneuploid Cells to PD-L1 Content Epithelial Cells. A threshold of a PD-L1-aneuploid-to-PD-L1-epithelial ratio for a sample may be employed to identify whether a malignant bladder-associated neoplasia is present, or likely to be present, in a subject from which the sample was derived. In some instances, the ratio may be employed to differentiate between high grade (HG) and low grade (LG) transitional cell carcinoma of the bladder (BC). Useful PD-L1-aneuploid-to-PD-L1-epithelial ratio thresholds may vary, e.g., depending on the sensitivity and/or specificity desired, and may range from 0.30 or more to 0.10 or less, including but not limited to e.g., 0.30 or less, less than 0.30, 0.28 or less, less than 0.28, 0.26 or less, less than 0.26, 0.24 or less, less than 0.24, 0.22 or less, less than 0.22, 0.20 or less, less than 0.20, 0.18 or less, less than 0.18, 0.16 or less, less than 0.16, 0.14 or less, less than 0.14, 0.12 or less, less than 0.12, 0.10 or less, or less than 0.10.

In some instances, a method of the present disclosure may include a threshold for an epithelial-aneuploid content value (also referred to as an “Aneuploid Content” derived feature), where such corresponds to the product of DNA index and Perct_Aneuploid Epithelial cells. Useful epithelial-aneuploid content value thresholds may vary, e.g., depending on the sensitivity and/or specificity desired, and may range from 0.20 or more to 0.05 or less, including but not limited to e.g., 0.20 or less, less than 0.20, 0.18 or less, less than 0.18, 0.16 or less, less than 0.16, 0.14 or less, less than 0.14, 0.12 or less, less than 0.12, 0.10 or less, less than 0.10, 0.09 or less, less than 0.09, 0.08 or less, less than 0.08, 0.06 or less, less than 0.06, 0.05 or less, or less than 0.05.

Subjects having samples identified as containing PD-L1(+) immune cells above a predetermined threshold may be more likely to be responsive to therapies directed at disrupting the PD-1/PD-L1 interaction. In some instances, by effectively quantifying the presence of PD-L1 expressing immune cells, and detecting the presence of such cells above a threshold level, the effectiveness of therapies targeting the PD-1/PD-L1 interaction may be determined. Subjects having samples identified as containing a PD-L1-aneuploid-to-PD-L1-epithelial ratio above a predetermined threshold may be more likely to be responsive to therapies directed at disrupting the PD-1/PD-L1 interaction. In some instances, by effectively quantifying PD-L1 expression, and detecting a PD-L1-aneuploid-to-PD-L1-epithelial ratio above a threshold level, the effectiveness of therapies targeting the PD-1/PD-L1 interaction may be determined. In some instances, the ratio may be employed to differentiate between high grade (HG) and low grade (LG) transitional cell carcinoma of the bladder (BC) and, in some such instances, identify and/or employ an appropriate therapy based on such determination. Thus, such methods may be utilized in making treatment decisions, including whether to treat a subject with a therapy targeting the PD-1/PD-L1 interaction.

Accordingly, the present disclosure includes methods of identifying whether a bladder-associated neoplasia in a subject is anti-PD-1/PD-L1 immunotherapy responsive. As used herein, anti-PD-1/PD-L1 immunotherapy responsive generally refers to the responsiveness of a neoplasia to a treatment targeting the interaction between PD-1 and PD-L1, including e.g., by using an antagonist to PD-1 and/or PD-L1. As such, a neoplasia identified, or a subject having an identified neoplasia, may be referred to as an anti-PD-1/PD-L1 immunotherapy responsive. Methods of the invention also include differentiating between high grade (HG) and low grade (LG) transitional cell carcinoma of the bladder (BC) based on the ratio.

Cytometric Assays

As summarized above, methods of the present disclosure include cytometrically assaying a labeled cell suspension. Various methods of cytometrically assaying a labeled cell suspension may find use in the herein described methods including but not limited to e.g., flow cytometrically assaying using a flow cytometer, cell cytometrically assaying a labeled cell suspension, e.g., by using a cell cytometer, and the like. Labeled cell suspension samples may be assayed for per cell PD-L1 expression. In some cases, additional cellular parameters, assayed cytometrically, may also find use in detecting cells of the instant disclosure. Accordingly, various methods of cytometrically assaying a labeled cell suspension to measure various cellular parameters may be employed.

In some embodiments, cytometrically assaying a cellular sample may be performed using flow cytometry. Flow cytometry is a methodology using multi-parameter data for identifying and distinguishing between different particle (e.g., cell) types i.e., particles that vary from one another in terms of label (wavelength, intensity), size, etc., in a fluid medium. In flow cytometrically analyzing a sample, an aliquot of the sample is first introduced into the flow path of the flow cytometer. When in the flow path, the cells in the sample are passed substantially one at a time through one or more sensing regions, where each of the cells is exposed separately and individually to a source of light at a single wavelength (or in some instances two or more distinct sources of light) and measurements of cellular parameters, e.g., light scatter parameters, and/or marker parameters, e.g., fluorescent emissions, as desired, are separately recorded for each cell. The data recorded for each cell is analyzed in real time or stored in a data storage and analysis means, such as a computer, for later analysis, as desired.

In flow cytometry-based methods, the cells are passed, in suspension, substantially one at a time in a flow path through one or more sensing regions where in each region each cell is illuminated by an energy source. The energy source may include an illuminator that emits light of a single wavelength, such as that provided by a laser (e.g., He/Ne or argon) or a mercury arc lamp or an LED with appropriate filters. For example, light at 488 nm may be used as a wavelength of emission in a flow cytometer having a single sensing region. For flow cytometers that emit light at two distinct wavelengths, additional wavelengths of emission light may be employed, where specific wavelengths of interest include, but are not limited to: 405 nm, 535 nm, 561 nm, 635 nm, 642 nm, and the like. Following excitation of a labeled specific binding member bound to a polypeptide by an energy source, the excited label emits fluorescence and the quantitative level of the polypeptide on each cell may be detected, by one or more fluorescence detectors, as it passes through the one or more sensing regions.

In flow cytometry, in addition to detecting fluorescent light emitted from cells labeled with fluorescent markers, detectors, e.g., light collectors, such as photomultiplier tubes (or “PMT”), an avalanche photodiode (APD), etc., are also used to record light that passes through each cell (generally referred to as forward light scatter), light that is reflected orthogonal to the direction of the flow of the cells through the sensing region (generally referred to as orthogonal or side light scatter) as the cells pass through the sensing region and is illuminated by the energy source. Each type of data that is obtained, e.g., forward light scatter (or FSC), orthogonal light scatter (SSC), and fluorescence emissions (FL1, FL2, etc.), comprise a separate parameter for each cell (or each “event”).

Flow cytometers may further include one or more electrical detectors. In certain embodiments, an electrical detector may be employed for detecting a disturbance caused by a particle or cell passing through an electrical field propagated across an aperture in the path of the particles/cells. Such flow cytometers having electrical detectors will contain a corresponding electrical energy emitting source that propagates an electrical field across the flow path or an aperture through which cells are directed. Any convenient electrical field and/or combination of fields with appropriate detector(s) may be used for the detection and/or measurement of particles (or cells) passing through the field including but not limited to, e.g., a direct current electrical field, alternating current electrical field, a radio-frequency field, and the like.

Flow cytometers further include data acquisition, analysis and recording means, such as a computer, wherein multiple data channels record data from each detector for each cell as it passes through the sensing region. The purpose of the analysis system is to classify and count cells wherein each cell presents itself as a set of digitized parameter values and to accumulate data for the sample as a whole.

A particular cell subpopulation of interest may be analyzed by “gating” based on the data collected for the entire population. To select an appropriate gate, the data is plotted so as to obtain appropriate separation of subpopulations, e.g., by adjusting the configuration of the instrument, including e.g., excitation parameters, collection parameters, compensation parameters, etc. In some instances, this procedure is done by plotting forward light scatter (FSC) vs. side (i.e., orthogonal) light scatter (SSC) on a two dimensional dot plot. The flow cytometer operator then selects the desired subpopulation of cells (i.e., those cells within the gate) and excludes cells which are not within the gate. Where desired, the operator may select the gate by drawing a line around the desired subpopulation using a cursor on a computer screen. Only those cells within the gate are then further analyzed by plotting the other parameters for these cells, such as fluorescence.

Any flow cytometer that is capable of obtaining fluorescence data, e.g., as described above, may be employed. Useful flow cytometers include those utilizing various different means of flowing a cell through the sensing region substantially one at a time including, e.g., a flow cell, a microfluidics chip, etc. Non-limiting examples of flow cytometer systems of interest are those available from commercial suppliers including but not limited to, e.g., Becton-Dickenson (Franklin Lakes, N.J.), Life Technologies (Grand Island, N.Y.), Acea Biosciences (San Diego, Calif.), Beckman-Coulter, Inc. (Indianapolis, Ind.), Bio-Rad Laboratories, Inc. (Hercules, Calif.), Cytonome, Inc. (Boston, Mass.), Amnis Corporation (Seattle, Wash.), EMD Millipore (Billerica, Mass.), Sony Biotechnology, Inc. (San Jose, Calif.), Stratedigm Corporation (San Jose, Calif.), Union Biometrica, Inc. (Holliston, Mass.), Cytek Development (Fremont, Calif.), Propel Labs, Inc. (Fort Collins, Colo.), Orflow Technologies (Ketchum, Id.), handyem inc. (Québec, Canada), Sysmex Corporation (Kobe, Japan), Partec Japan, Inc. (Tsuchiura, Japan), Bay bioscience (Kobe, Japan), Furukawa Electric Co. Ltd. (Tokyo, Japan), On-chip Biotechnologies Co., Ltd (Tokyo, Japan), Apogee Flow Systems Ltd. (Hertfordshire, United Kingdom), and the like.

In some embodiments, cytometrically assaying a cellular sample may be performed using a cell cytometer. As used herein, the term “cell cytometer” (also referred to as an “imaging cytometer” or “automated imaging cytometer”) generally refers to an automated or semi-automated cell imaging device capable of imaging cells deposited on or in an imaging vessel to collect data on all or most of the cells of a sample. In cell cytometry, imaging may be performed according to a variety of different methods. In some instances, a cell cytometer may collect a widefield image at low magnification (e.g., 5×, 10×, etc.) of the cells present on or in an imaging vessel to identify the location of the cells and/or screen the cells for a particular parameter (e.g., size, shape, color, fluorescence, etc.). After identifying the location of the cells a cell cytometer may proceed to collect higher magnification (e.g., 20×, 40×, 60×, 100×, etc.) images of all or a portion of the identified cells, e.g., in a targeted manner.

In other instances, a cell cytometer may image cells present on or in an imaging vessel by scanning the imaging vessel. Scanning may be performed at low or high magnification. In some instances, scanning is performed at high magnification to capture images of all or most of the cells. In some instances, scanning is performed at low magnification to identify the location of the cells on or in the imaging vessel. After identifying the location of the cells a cell cytometer may proceed to collect higher magnification images of all or a portion of the identified cells, e.g., in a targeted manner, or may rescan the located cells at high magnification.

The imaging vessels used in cell cytometer systems will vary. In some instances, commonly used laboratory imaging devices such as e.g., microscope slides, may serve as an imaging vessel in a cell cytometer system. In some instances, a cell cytometer imaging vessel may be specifically designed for use with a particular cell cytometer. Useful imaging vessels include but are not limited to e.g., slides (e.g., microscope slides), dishes (e.g., glass bottom imaging dishes), plates (e.g., multi-well imaging plates), etc. Imaging vessels will generally have optical properties amendable to microscopy, e.g., optical clarity, in at least a portion of the vessel. Imaging vessels may or may not have individual compartments. For example, a microscope slide utilized as an imaging vessel does not generally have individual compartments and cells deposited on a slide may be spread about the surface of the slide. Alternatively, a multi-well imaging plate utilized as an imaging vessel does have individual compartments (i.e., wells) into which one or more cells may be deposited.

Cell cytometers include an imaging component such as, e.g., an automated microscope. The imaging component of a cell cytometer may include one or more objectives of various magnification power (e.g., 5×, 10×, 20×, 40× 60×, 100×, etc.) for collecting light transmitted, reflected or emitted from the object (e.g., cell) being imaged. Light collected by the objective will generally be processed through one or more dichroic mirrors, filters or lenses before being directed to an image capture device.

Suitable image capturing devices may include one or more digital cameras (including color and monochrome cameras) capable of capturing a digital image and a means of storing the digital image and/or transferring the image to attached image processing circuitry or to an attached storage device for later transfer to image processing circuitry. Suitable digital color cameras will vary and will generally include any digital camera (e.g., with one or more CCD or CMOS sensors). Suitable digital cameras include but are not limited to e.g., custom built digital cameras, consumer grade digital color cameras (e.g., consumer grade digital color cameras converted for microscopic use) and those digital microscopy color cameras commercially available from various manufactures including but not limited to e.g., Dino-Eye, Dino-Lite, Jenoptik ProgRes, KoPa, Leica, Motic, Olympus, Omano, OptixCam, PixelLINK, Zeiss, etc.

Cell cytometers further include data acquisition, analysis and recording means, such as a computer, wherein one or more data channels record data from one or more image capture devices for each cell or most of the cells of the imaging vessel. The purpose of the analysis system is to classify and count cells wherein each cell presents itself as a set of digitized parameter values and to accumulate data for the sample as a whole. In some cases, cell cytometers record images of each cell and may be connected to a user interface where such images may be reviewed by a user of the device.

Cell cytometer based methods for detecting cells expressing a particular polypeptide may include contacting the cells of a sample with a fluorescent labeled specific binding member and detecting fluorescently labeled cells by imaging using the cell cytometer. As described in more detail elsewhere herein, the fluorescence of each labeled cell may be cytometrically quantified to identify the per cell expression of a particular polypeptide, e.g., to detect whether a cell expresses the polypeptide above a predetermined threshold.

Any cell cytometer that is capable of obtaining fluorescence data, e.g., as described above, may be employed. Useful cell cytometers include those utilizing various different means of automated cell cytometric imaging to analyze all or most of the cells of a sample. Non-limiting examples of cell cytometer systems of interest are those available from commercial suppliers including but not limited to, e.g., Nexcelom Bioscience LLC (Lawrence, Mass.), Molecular Devices, LLC (Sunnyvale, Calif.), Thorlabs Inc. (Newton, N.J.), TTP Labtech Ltd. (United Kingdom), and the like.

The present disclosure also includes cytometric devices that include a bladder-associated sample, including e.g., those samples described herein. For example, in some instances, a cytometric device may include a labeled-bladder associated sample, including but not limited to e.g., where such a sample is labeled with one or more of a labeled binding member specific for PD-L1, a DNA labeling reagent, at least one labeled binding member specific for immune cells, at least one labeled binding member specific for epithelial cells, or a combination thereof. For example, in some instances, a sample present in a cytometric device may include an anti-PD-L1 antibody and one or more of an anti-CD45 antibody and an anti-pan-cytokeratin antibody. In some instances, the cytometric device that includes such a sample may be a flow cytometer. In some instances, the cytometric device that includes such a sample may be a cell cytometer.

Methods of the instant disclosure include cytometrically quantifying per cell expression levels of particular polypeptides to identify cells expressing the polypeptide above a predetermined threshold. Methods of the instant disclosure may include cytometrically quantifying per cell PD-L1 expression to identify one or more cells expressing PD-L1 above a predetermined threshold. However, the levels of other markers besides PD-L1 may also be assessed in the herein described methods including e.g., cell cycle associated expression products (e.g., cell cycle associated RNAs, cell cycle associated polypeptides, etc.), immune-related expression products (e.g., immune-related RNAs, immune-related polypeptides, etc.), DNA content, cell-type associated expression products (e.g., epithelial cell related RNAs, epithelial polypeptide markers, etc.), and the like. Detection of cells having a level of a biomarker, e.g., above or below a predetermined threshold, or not having such other markers may serve to identify further cell parameters useful in the herein described methods.

Predetermined thresholds may find use in identifying cells based on their expression of a particular polypeptide (PD-L1, immune cell-type markers, epithelial cell type markers, etc.), as described above, or other cellular parameters including but not limited to e.g., cell cycle markers, aneuploidy markers, and the like.

Predetermined thresholds for polypeptide expression useful in the instant disclosure will vary depending on the polypeptide detected and the particular context. In some instances, a predetermined threshold for per cell polypeptide expression may be expressed as a number of molecules of the polypeptide per cell, including but not limited to e.g., a threshold of 10 molecules per cell, a threshold of 20 molecules per cell, a threshold of 30 molecules per cell, a threshold of 40 molecules per cell, a threshold of 50 molecules per cell, a threshold of 60 molecules per cell, a threshold of 70 molecules per cell, a threshold of 80 molecules per cell, a threshold of 90 molecules per cell, a threshold of 100 molecules per cell, a threshold of 200 molecules per cell, a threshold of 300 molecules per cell, a threshold of 400 molecules per cell, a threshold of 500 molecules per cell, a threshold of 600 molecules per cell, a threshold of 700 molecules per cell, a threshold of 800 molecules per cell, a threshold of 900 molecules per cell, a threshold of 1000 molecules per cell, a threshold of 1100 molecules per cell, a threshold of 1200 molecules per cell, a threshold of 1300 molecules per cell, a threshold of 1400 molecules per cell, a threshold of 1500 molecules per cell, a threshold of 1600 molecules per cell, a threshold of 1700 molecules per cell, a threshold of 1800 molecules per cell, a threshold of 1900 molecules per cell, a threshold of 2000 molecules per cell, etc.

In other instances, a predetermined threshold may be a relative level of a marker. Relative levels of a marker may be determined by a variety of means including e.g., determined by making a comparison of the levels of expression of a marker in two separate populations of cells known to differ in their level of the subject marker. For example, a first cell population known to have a high level of Marker X is measured, e.g., on a cytometer, and compared to a second cell population, known to have a low level of Marker X and the comparison is used to determine a threshold level that may be used to categorize cells as either having a low or a high level of Marker X.

Relative levels of a marker may be determined by making a comparison of the levels of marker within a population of cells, e.g., a population of cells of unknown levels of Marker X or a population of cells suspected of containing subpopulations of cells having different levels of Marker X. For example, the level of Marker X is measured on a cytometer of at least a sufficient number of cells such that the measurements may be plotted, e.g., on a histogram, and separation between two or more subpopulations of cells is revealed based on individual cell levels of Marker X. Accordingly, the cytometer operator may then determine a threshold level between the subpopulations that may be used to categorize cells as belonging to a particular subpopulation, e.g., a subpopulation having a low level of Marker X or a subpopulation having high level of Marker X.

In some instances, a threshold may be based on the limit of detection of the cytometer. For example, cells of a population of cells may be identified as having a particular marker (i.e., being positive for a particular marker) if the cells have any detectable level of a particular marker. Likewise, cells of a population of cells may be identified as not having a particular marker (i.e., being negative for a particular marker) if the cells do not have a detectable level of a particular marker. Accordingly, the detection level of the cytometer may be used to determine the marker threshold, as desired.

In some instances, a threshold may be based on previously determined marker levels, e.g., from previously performed control experiments or previously acquired reference expression levels. For example, marker levels determined in previously analyzed samples may be used to determine marker threshold levels. In some instances, marker levels expected of cells obtained from healthy subjects may be used to determine normal marker levels such that a marker threshold that is representative of the normal marker range may be determined. In such instances, marker expression outside, i.e., above or below, the normal marker range is considered to be either above or below the particular marker threshold. In some instances, use of such previously determined marker levels or previously determined threshold levels allows analysis of cells and the identification of cellular subpopulations in the absence of a control or reference cellular sample.

In some instances, a threshold level of a particular parameter may be determined by a comparison between the levels of the parameter observed in known healthy control samples versus the levels of the parameter observed in known neoplastic samples, or a subset thereof including e.g., metastatic neoplastic samples. For example, the parameter may be analyzed in both healthy samples and neoplastic samples and a threshold level of the parameter may be determined that retrospectively differentiates the healthy and neoplastic samples. The determined threshold may then be employed in further analyses as a predetermined threshold for identifying a sample as, e.g., healthy, normal, neoplastic, metastatic, non-metastatic, etc.

In some instances, a predetermined threshold of a parameter may not individually differentiate samples (e.g., normal/healthy vs. neoplastic, non-metastatic vs. metastatic, etc.) alone, but may be employed in combination with one or more additional predetermined thresholds of one or more additional parameters to differentiate samples. Such combinations of predetermined thresholds may be determined by performing comparisons between the levels of the parameters observed in known healthy control samples versus the levels of the parameters observed in known neoplastic samples, or a subset thereof including e.g., metastatic neoplastic samples. Next the observed parameters may be statistically compared in one or more combinations to determine a combination of thresholds for the parameters that collectively differentiates the healthy, neoplastic, metastatic, and/or non-metastatic samples with an appropriate degree of reliability. The determined thresholds may then be employed in further analyses as predetermined thresholds that, in combination, identify a sample as, e.g., healthy, normal, neoplastic, metastatic, non-metastatic, etc.

In some instances, a threshold may be redetermined or reoptimized, e.g., when elements of the assay are altered, e.g., a new reagent is employed. Such modification of thresholds employed in the methods of the present disclosure, where necessary, are within the skill of the relevant ordinarily skilled artisan in view of the instant disclosure.

As noted above, methods of the instant disclosure may include assaying cell cycle parameters. Useful cell cycle parameters include but are not limited to e.g., proliferation, cell cycle phase (G₁, G₂, M, G₂-M, S, G₀, post G₁, and the like), etc. Cell cycle parameters may be assessed on a per cell basis, including e.g., identifying whether a cell is proliferative, identifying the cell cycle phase of a cell, etc. Any convenient means of determining a cell cycle parameter of a cell may be employed in the subject methods. In some instances, a method may not only quantify a particular cell type but also determine whether the quantified cell type is proliferative including e.g., the number or percent of proliferative cells within the quantified cell type. In some instances, a method may not only quantify a cell type but also determine whether the quantified cell type is proliferative including e.g., the number or percent of proliferative cells within the quantified cell type.

In some instances, assaying the cell cycle of a cell may include determining the DNA content of the cell (i.e., the per cell DNA content). Various methods may be employed for assaying the cell cycle of a cell by determining the per cell DNA content. In some instances, a DNA labeling reagent (e.g., a nucleic acid dye or stain that contains intrinsic fluorescence) may be employed to label the DNA of the cell and the amount of DNA may be quantified based on the measuring the intensity of the label. Depending on the method of cytometry employed in the method, DNA content may be used to assess cell cycle in various ways. In one embodiment, e.g., regardless of the type of cytometry employed (e.g., flow cytometry, cell cytometry, etc.), the fluorescent intensity of cells labeled with a DNA labeling reagent may analyzed on the cytometer and plotted on a histogram. From the histogram the relative amount of DNA content may be determined for each cell allowing for the identification of the cell cycle phase of each cell. In some instances, such a histogram may represent a cytometric cell cycle profile, also referred to as a cytometric DNA profile.

In some instances, assaying the cell cycle of a cell may include assaying an expressed cell cycle marker (also referred to as a cell cycle biomarker). Expressed cell cycle markers, as used herein, refer to those cellular markers (e.g., cell surface markers and intracellular markers) that are specifically expressed or absent during one or more particular phases of the cell cycle. Accordingly, a labeled binding member specific for an expressed cell cycle marker include to those specific binding members that bind components of the cell cycle machinery of the cell. Cell cycle biomarkers may be useful, in some instances, in determining the cell cycle phase of or determining whether or not a cell is proliferative. Cell cycle biomarkers useful in the methods described herein will vary depending on the particular assay and/or the particular cell type and/or cell population to be detected. In some instances, cell cycle biomarkers that may find use in the methods described herein include but are not limited to, e.g., Ki67, cyclin D1, cyclin E, phosphorylated histone H3, and the like. Expressed cell cycle markers may be detected in various ways. For example, an expressed cell cycle biomarker may be detected at the protein level, e.g., through the use of a labeled specific binding member specific for the cell cycle biomarker protein. In some instances, an expressed cell cycle biomarker may be detected at the RNA level, e.g., through the use of a labeled specific binding member specific for the cell cycle biomarker RNA.

As noted above, methods of the instant disclosure may include assaying aneuploidy. Any convenient method of measuring aneuploidy cytometrically may be employed in the subject methods. In some instances, a cell may be identified as aneuploid based on the measured DNA content of the cell where an aneuploid cell will generally have an abnormally high level of DNA content representing duplication of all or a portion of the cell's genome. Similar methods to those described above for assessing DNA content in regards to cell cycle assessments may be employed for detecting aneuploidy. In some instances, relative DNA content greater than or equal to a threshold DNA content value for a normal cell may indicate that the cell is aneuploid where the threshold may be greater than or equal to (≥) 1.05 times the DNA content of a normal cell including but not limited to, e.g., ≥1.06 times, ≥1.07 times, ≥1.08 times, ≥1.09 times, ≥1.10 times, ≥1.11 times, ≥1.12 times and ≥1.13 times the DNA content of a normal cell.

In some instances, chromosome specific probes or gene specific probes may be employed to assess aneuploidy. For example, fluorescent in situ hybridization (FISH) using gene specific or chromosome specific probes may be employed to determine the overall ploidy of a cell or to detect the duplication of a particular gene or chromosome. For example, in a diploid organism, the presence of more than two probes for a specific gene or a specific chromosome may indicate that the subject cell is aneuploid.

Ploidy assessments (e.g., assessing the ploidy of a cell, including e.g., whether a cell is aneuploid, diploid, etc.) may be employed in the subject methods for various purposes. For example, in some instances, a ploidy assessment may be employed to determine whether cells of a population are aneuploid or diploid, including e.g., to determine whether an epithelial cell is aneuploid or diploid, whether an immune cell is aneuploid or diploid, or the like. In some instances, a ploidy assessment may inform other characteristics of the sample and/or the subject, e.g., by a relationship between the ploidy status of a detected cell and other cell types that may be present in the subject. For example, in some instances, the identification of certain aneuploid cells may be indicative and/or predictive of the presence of a malignant neoplastic cell type in a subject other than the detected aneuploid cell type, e.g., the presence of aneuploid epithelial cells in a bladder-associated sample from the subject may be indicative of, alone or in combination with other factors, the presence of malignant tumor cells in the subject. In some instances, a ploidy assessment may be used in generating a derived feature, examples of which are described herein, that includes one or more other measured and/or derived features combined, according to a mathematical relationship, with the ploidy assessment. In some instances, the presence of aneuploid immune cells, alone or in combination with other factors, in a bladder-associated sample may be indicative of the presence of malignant tumor cells in the subject. In some instances, the presence of a derived feature that includes assessment of aneuploid cells, alone or in combination with other factors, in a bladder-associated sample may be indicative of the presence of malignant tumor cells in the subject.

Assessments of cellular parameters may be used in the subject methods to detect cells that have one or more characteristics detected by measuring the described parameters. For example, in some instances, a detected cell may be determined to be an aneuploid cell, e.g., based on one or more assessed aneuploidy parameters of the cell. In some instances, a detected cell may be determined to be a proliferative cell, e.g., based on one or more assessed cell cycle parameters of the cell. Cells may be detected as having a combination of characteristics detected by measuring the described parameters. For example, a cell may be determined to be both proliferative and aneuploid, both epithelial and aneuploid, both PD-L1 positive and epithelial, or PD-L1 positive, epithelial and aneuploid, etc. In addition, the absence of a characteristic may also be used when detecting a particular cell including e.g., where the cell is not proliferative, where the cell is not aneuploid, where the cell is not PD-L1 positive (i.e., PD-L1 negative), etc. In some instances, a detected cell may have one characteristic and lack another, e.g., where the cell is proliferative but not aneuploid, where the cell is aneuploid but no proliferative, where the cell is epithelial but not PD-L1 positive, where the cell is aneuploid but not PD-L1 positive, etc. Any combination of the herein described parameters may find use in the methods of the present disclosure.

As an example, useful combinations of determined parameters may include per cell PD-L1 expression combined with ploidy status, including e.g., where cells or a population of cells are detected that include per cell PD-L1 expression above a predetermined threshold and an aneuploid ploidy status. Useful combinations may also include per cell PD-L1 expression combined with a DNA content (i.e., DNA index) or cell cycle determination, PD-L1 expression combined with epithelial marker expression, ploidy status combined with epithelial marker expression, PD-L1 expression combined with ploidy status, and the like.

In some instances, the methods of the instant disclosure may further include determining whether a subject cell is or is not an immune cell. Various methods may be employed for determining whether a subject cell is or is not an immune cell including e.g., through detecting the presence or absence of one or more immune cell markers, e.g., through contacting the cell with a labeled specific binding member specific for an immune cell marker.

For example, in some instances, an immune cell may be detected based on the presence of labeling with an immune cell specific binding member added to the cell suspension. In some instances, a non-immune cell may be detected based on an absence of labeling with an immune cell specific binding member added to the cell suspension. Accordingly, in some instances, the method may further include determining that the identified cell is or is not an immune cell, e.g., by contacting the cell suspension with one or more labeled specific binding members for immune cells.

Accordingly, in some instances, a cell and/or a population of cells may be identified as being positive or negative for a particular immune cell marker or having a level of expression of an immune cell marker that is above or below a predetermined threshold indicative that the cell is, in fact, an immune cell or not an immune cell or a particular type of immune cell. Useful immune cell markers include but are not limited to e.g., CD114, CD117, CD11a, CD11b, CD14, CD15, CD16, CD182, CD19, CD20, CD22, CD24, CD25, CD3, CD30, CD31, CD34, CD38, CD4, CD45, CD56, CD61, CD8, CD91, Foxp3, and the like. Accordingly, in some instances, a detected cell may be further characterized as having or lacking expression, or having expression above or below a predetermined threshold, of one or more immune cell markers, e.g., as detected using an antibody to an immune cell marker including e.g., those listed above.

In some instances, a cell may be assayed in the herein described methods for expression of a combination of immune cell markers including but not limited to e.g., any combination of the here described markers.

In some instances, a cell may be detected based on expressing PD-L1 above a predetermined threshold and labeling with an immune cell specific binding member added to the cell suspension. Accordingly, in some instances, the method may include determining that the identified cell is an immune cell, e.g., by contacting the cell suspension with one or more labeled specific binding members for immune cells.

Accordingly, in some instances, a cell and/or a population of cells may be identified as being positive for a particular immune cell marker or having a level of expression of an immune cell marker that is above a predetermined threshold indicative that the cell is, in fact, an immune cell or a particular type of immune cell. Useful immune cell markers, e.g., for identifying a PD-L1 expressing cell as an immune cell include but are not limited to e.g., CD114, CD117, CD11a, CD11b, CD14, CD15, CD16, CD182, CD19, CD20, CD22, CD24, CD25, CD3, CD30, CD31, CD34, CD38, CD4, CD45, CD56, CD61, CD8, CD91, Foxp3, and the like. Accordingly, in some instances, a detected cell may be further characterized as having expression of or having expression of above a predetermined threshold of one or more immune cell markers, e.g., as detected using an antibody to an immune cell marker including e.g., those listed above.

In some instances, a cell may be detected based on expressing PD-L1 above a predetermined threshold and labeling with an epithelial cell specific binding member added to the cell suspension. Accordingly, in some instances, the method may include determining that the identified cell is an epithelial cell, e.g., by contacting the cell suspension with one or more labeled specific binding members for epithelial cells.

Accordingly, in some instances, a cell and/or a population of cells may be identified as being positive for a particular epithelial cell marker or having a level of expression of an epithelial cell marker that is above a predetermined threshold indicative that the cell is, in fact, an epithelial cell. Useful labeled binding members specific for epithelial cells include but are not limited to those described herein, including e.g., anti-pan-cytokeratin antibodies and the like. Accordingly, in some instances, a detected cell may be further characterized as having expression of or having expression of above a predetermined threshold of one or more epithelial cell markers, e.g., as detected using an antibody to an epithelial cell marker.

Methods of the instant disclosure include the detection of a cell expressing PD-L1 above a predetermined threshold. In some instances, the instant methods may encompass the detection of a plurality of cells expressing PD-L1 above the predetermined threshold. For example, in some instances, the size of a population of cells expressing PD-L1 above the predetermined threshold may be determined. Quantification of the size of a population of cells expressing PD-L1 above the predetermined threshold may be measured cytometrically. For example, in some instances, a flow cytometer may be used to count the number of cells that express PD-L1 above a predetermined threshold. In some instances, a cell cytometer may be used to count the number of cells that express PD-L1 above a predetermined threshold. By counting the number of cells, the size of the PD-L1 expressing population may be determined.

Samples

As summarized above, methods of the instant disclosure include detecting per cell PD-L1 expression, including detecting whether an immune cell that expresses PD-L1 above a predetermined threshold is present in a sample and/or detecting a PD-L1-aneuploid-to-PD-L1-epithelial ratio of a sample that is above a predetermined threshold. The herein described methods are applicable to various samples where useful samples include bladder-associated samples. Liquid samples may be assessed in the subject methods, including where the sample is a liquid, such as urine, cytology effluent (e.g., bladder-irrigation fluid), etc., or a liquid cellular sample derived from the solid or semi-solid sample, such as a biopsy sample.

Samples of the present disclosure include labeled bladder-associated samples. Such samples may include cells expressing PD-L1, e.g., an immune cell expressing PD-L1, an epithelial cell expressing PD-L1, etc., and a labeled binding member specific for PD-L1 noncovalently bound to the cell. In some instances, such samples may further include at least one labeled binding member specific for immune cells, including e.g., where the labeled binding member specific for immune cells is noncovalently bound to the immune cell. Various labeled binding members specific for immune cells may be employed, including but not limited to e.g., those described herein, including e.g., anti-CD45 antibodies. In some instances, such samples may further include at least one labeled binding member specific for epithelial cells, including e.g., where the labeled binding member specific for epithelial cells is noncovalently bound to the epithelial cell. Various labeled binding members specific for epithelial cells may be employed, including but not limited to e.g., those described herein, including e.g., anti-pan-cytokeratin antibodies.

Samples of the present disclosure may, in some instances, include further labeling reagents, i.e., labeling reagents in addition to PD-L1 specific binding members. Useful further labeling reagents that may be present in samples of the present disclosure include but are not limited to DNA labeling reagents, labeled binding members specific for epithelial cells, and the like. Useful DNA labeling reagents may include but are not limited to e.g., those described herein. Useful labeled binding members specific for epithelial cells include but are not limited to those described herein, including e.g., anti-pan-cytokeratin antibodies and the like.

Samples of the present disclosure, including samples employed in the herein described methods, may be a liquid sample obtained from a subject. Useful liquid samples obtained from subject include urine, cytology effluent (e.g., bladder irrigation fluid), and the like. Liquid samples may be tested for and/or determined to have hematuria, including e.g., microscopic hematuria. Liquid samples, including urine, cytology effluent (e.g., bladder-irrigation fluid), etc., may be collected from a subject by any convenient and appropriate means, including but not limited to e.g., where the sample is collected via micturition or catheterization.

Samples of the present disclosure, including samples employed in the herein described methods, may be a liquid sample prepared from a solid tissue sample obtained from a subject. Various solid tissue samples may be employed as appropriate, including but not limited to e.g., where the sample is a biopsy sample, including a biopsy sample taken from a subject's urinary tract. Useful biopsy samples include bladder biopsy samples and the like. Solid tissue samples may be rendered into a liquid form prior to analysis in a method of the present disclosure, including e.g., where the cells of the solid tissue sample are dissociated. As such, methods of the present disclosure may include dissociating the solid tissue sample to generate the liquid cellular sample.

Samples, including liquid samples obtained from a subject and dissociated solid tissue samples, may or may not be enriched for cells that are analyzed in the methods of the present disclosure. For example, in some instances, a sample may be analyzed directly without enriching for cells that may be present in the sample. In some instances, a sample may be processed, such as e.g., using centrifugation, filtration, or other appropriate method or combination thereof, to enrich the sample for cells. For example, a urine sample or, cytology effluent (e.g., bladder-irrigation fluid) may be centrifuged to produce a cell pellet. The supernatant may be aspirated, and the cell pellet may be resuspended in a volume of liquid less than the aspirate order to enrich for the cells present in the sample.

As described in more detail below, cells of the sample may be fixed, including where fixation is performed at one or more various points in the procedure. For example, cells may be fixed before, during or after the cells are contacted with one or more labeling reagents. In some instances, cells are fixed before, during, or after enrichment. Various fixation reagents, as described below, may be employed.

As summarized above, cells analyzed in the methods of the present disclosure may be labeled. Accordingly, methods of the present disclosure may be contacted with one or more labeling reagents, including but not limited to e.g., the labeling reagents described herein. As an example, the instant methods may include contacting a bladder-associated sample with a labeled binding member specific for PD-L1 to generate a labeled cell suspension. In some instances, the sample may be contacted with further labeling reagents, including but not limited to e.g., DNA labeling reagents, labeled binding members specific for epithelial cells, labeled binding members specific for immune cells, and the like and/or a combination thereof.

Methods of the present disclosure include identifying whether a subject from which a sample is obtained, based on the analysis of the sample, has a neoplasm. Neoplasms include benign neoplasms, in situ neoplasms, malignant neoplasms, and neoplasms of uncertain or unknown behavior. Malignant neoplasms include cancer and accordingly the subject methods may include detecting whether a cancer cell is present in a sample and/or identifying whether a cancer is present in a subject from which the sample is derived.

As summarized above, in some instances, a bladder-associated sample obtained from a subject may be obtained as a liquid from the subject, including e.g., urine, cytology effluent (e.g., bladder-irrigation fluid), etc. Such liquids may not be expected to contain a high number of neoplastic cells of a subject's neoplasm. In contrast, bladder-associated samples obtained directly from a tissue known or suspected of being a neoplasm, e.g., from a biopsy, may contain a high number of neoplastic cells.

Samples that contain or may contain neoplasia cells may be obtained using any convenient sample collection method, including but not limited to those biopsy methods for obtaining solid tissue biopsies and biopsy aspirates. In some instances, a sample containing neoplastic cells may be obtained as part of a separate medical procedure performed for a purpose other than obtaining the sample, including but not limited to a surgical procedure. In other instances, a sample containing neoplastic cells may be obtained independently, e.g., not as part of a separate medical procedure. Sample collection methods will vary and will depend upon, e.g., whether the collection is or is not performed as part of an additional medical procedure, the particular type of sample to be obtained, the primary purpose for obtaining the sample and/or the method by which the sample is to be processed and/or analyzed.

Samples used in the methods of the present disclosure may be collected by any convenient means. In some instances, a neoplasia sample is prepared from a biopsy. Depending on the type of cancer and/or the type of biopsy performed the sample may be prepared from a solid tissue biopsy or a liquid biopsy.

In some instances, a sample may be prepared from a surgical biopsy. Any convenient and appropriate technique for surgical biopsy may be utilized for collection of a sample to be assessed according to the methods described herein including but not limited to, e.g., excisional biopsy, incisional biopsy, wire localization biopsy, and the like. In some instances, a surgical biopsy may be obtained as a part of a surgical procedure which has a primary purpose other than obtaining the sample, e.g., including but not limited to tumor resection, and the like.

In some instances, a sample may be obtained by a needle biopsy. Any convenient and appropriate technique for needle biopsy may be utilized for collection of a sample to be analyzed according to the methods described herein including but not limited to, e.g., fine needle aspiration (FNA), core needle biopsy, stereotactic core biopsy, vacuum assisted biopsy, and the like.

According to the particular biopsy method employed and depending on the specifics of a particular subject and/or a subject's particular lesion one biopsy or multiple biopsies may be performed. For example, in some instances, a single biopsy, e.g., a single FNA biopsy or a single core needle biopsy, may be performed to sufficiently sample a particular subject or a particular subject's lesion. In other instances, multiple biopsies, e.g., multiple FNA biopsies or multiple core needle biopsies, may be performed for the collection of a single sample or multiple samples from a subject or a subject's lesion. In instances where multiple biopsies are collected the actual number of biopsies will vary depending on the particular subject and/or the particular lesion or lesions of the subject and, as such, may range from 2 to 10 or more biopsies, including but not limited to, e.g., 2 biopsies, 3 biopsies, 4 biopsies, 5 biopsies, 6 biopsies, 7 biopsies, 8 biopsies, 9 biopsies, 10 biopsies, etc. Multiple biopsies may be collected in a co-timely manner or may be collected over a predetermined period of time, e.g., as part of a surveillance protocol.

Samples collected according to the methods described herein may be solid, semi-solid, or liquid samples. For example, in some instances, by nature of the collection technique utilized, e.g., techniques that cause the dissociation or aspiration of cells, the collected sample may be a liquid sample upon collection. In other instances, by nature of the collection technique utilized, e.g., surgical collection or core sample collection, the collected sample may be a solid or semi-solid sample upon collection. In embodiments where the collected sample is a solid or semi-solid sample the cells of the sample may be dissociated to form a liquid sample following collection. Methods of dissociating solid and semi-solid tissue samples include but are not limited to mechanical dissociation, chemical dissociation, enzymatic dissociation, and combinations thereof.

In some instances, solid samples may be subjected to mechanical homogenization. Any convenient method of mechanical homogenization may find use preparing a solid tissue sample for downstream steps including but not limited homogenization performed using a commercially available homogenization device including e.g., those available from IncellDx (Menlo Park, Calif.), such as e.g., those provided with the incelPREP (IncellDx, Inc) kit, Claremont BioSolutions (Upland, Calif.) including e.g., the microHomogenizer (Claremont BioSolutions), the microDisruptor (Claremont BioSolutions), and the like. Mechanical homogenization may be performed in any suitable solution, including e.g., a buffer. In some instances, mechanical homogenization may be combined with chemical or enzymatic homogenization. In some instances, a fixation reagent is added during homogenization. In some instances, a fixation reagent is added following, including immediately following, homogenization. Fixation reagents, described in more detail below, that may be added following homogenization include but are not limited to e.g., the incelPREP (IncellDx, Inc). In some instances, a fixation solution may be a combination fixation/permeabilization reagent.

In some instances, the fixative used in preparing the labeled cell suspension sample provides for the ability to cytometrically separate PD-L1 expressing cell from PD-L1 non-expressing cells, including but not limited to e.g., to effectively cytometrically separate cells having a per cell PD-L1 expression level above a predetermined threshold from those having a per cell PD-L1 expression level below the predetermined threshold.

Upon collection or preparation of the sample, e.g., enrichment, dissociation and/or homogenization, the cells of the resultant liquid cell suspension of may be fixed and/or permeabilized as desired. As such, aspects of the methods may include fixing the cells of the suspension by contacting the sample with a suitable fixation reagent. Fixation reagents of interest are those that fix the cells at a desired time-point. Any convenient fixation reagent may be employed, where suitable fixation reagents include, but are not limited to mildly cross-linking agents. In some instances, a mildly cross-linking agent may be a formaldehyde-based fixative including but not limited to e.g., formaldehyde, paraformaldehyde, formaldehyde/acetone, IncelIMAX (IncelIDx, Inc), etc. In some instances, an alcohol-based fixative may be employed including but not limited to e.g., methanol/acetone, ethanol, etc. In some instances, formaldehyde-based fixatives may be used at a final concentration of about 1 to 2%.

In some instances, the cells in the sample are permeabilized by contacting the cells with a permeabilizing reagent. Permeabilizing reagents of interest are reagents that allow the labeled biomarker probes, e.g., as described in greater detail below, to access to the intracellular environment. Any convenient permeabilizing reagent may be employed, where suitable reagents include, but are not limited to: mild detergents, such as Triton X-100, NP-40, saponin, etc.; methanol, and the like.

Samples used in the methods of the present disclosure are assayed cytometrically. Accordingly, in some instances, a sample may be a liquid sample and/or processed to generate a cell suspension suitable for cytometric assays. Processing to generate sample suitable for cytometric assays may include e.g., any individual step or combination of the steps described above including e.g., cell enrichment, homogenization, dissociation, fixation, permeabilization, etc. The amount of processing required will depend on various factors including the source of the sample where solid tissue samples will generally require more processing that a liquid sample. For example, processing of a liquid sample, e.g., a urine sample or a cytology effluent (e.g., bladder-irrigation fluid) sample, may not require homogenization or dissociation and thus may only require fixation and/or permeabilization and/or cell enrichment/collection, etc., as desired.

The cells of a cell suspension sample will generally be labeled with one or more labeled specific binding members. For example, methods of the present disclosure will generally include contacting a cell suspension sample with a labeled specific binding member specific for PD-L1. Other specific binding members and other labeling reagents may find use in the subject methods for labeling various aspects of a cell or cells of a population as described herein including but not limited to e.g., a maker (e.g., an immune cell marker, an epithelial cell marker, etc.), the nucleus of the cell, etc. Such regents are described in more detail below.

Contacting, e.g., contacting a cell of a cell suspension with a specific binding member may be carried out by any convenient and appropriate means. In some instances, a cell of a cell suspension may be contacted with a specific binding member by adding an aliquot of the specific binding member to the cell suspension. A contacted cell suspension may be incubated and/or post-fixed as desired.

Reagents

A summarized above, the instant methods include detecting per cell PD-L1 expression in bladder-associated samples. In some instances, the methods include the detection of an immune cell expressing PD-L1 in a bladder-associated sample and/or detecting a PD-L1-aneuploid-to-PD-L1-epithelial ratio of a sample that is above a predetermined threshold. In some instances, the methods may include quantifying PD-L1 expression and/or detecting a cell, where the cell expresses a per cell level of PD-L1 above a predetermined threshold. Thus, the methods may include various reagents useful in practicing such methods. For example, the instant methods may generally include a reagent for quantifying PD-L1 expression and/or detecting a cell expressing PD-L1 above a predetermined threshold, including where such a reagent includes a labeled specific binding member reagent suitable for cytometric assays to be performed.

In order to effectively cytometrically quantify the per cell level of a particular polypeptide a direct correlation between the amount of fluorescence measured from the labeled specific binding member and the number of polypeptides bound by the specific binding member may be desired. In some instances, the amount of fluorescence emitted by the labeled specific binding member is linearly correlated to the number of polypeptides bound by the labeled specific binding member. Generally, but not exclusively, a labeled specific binding member will bind one molecule of the target polypeptide. As such, in some instances, there may be a one-to-one correlation between the amount of fluorescence detected from a plurality of labeled specific binding members bound to polypeptides on the surface of a cell and the number of the polypeptides expressed by the cell. Accordingly, in instances where per cell expression of a polypeptide is quantified cytometrically, the labeled specific binding members used may all uniformly have the same amount of attached label such that each specific binding member emits essentially the same amount of fluorescence. For example, a labeled specific binding member may have a single attached label or a single fluorescent moiety. Alternatively, a labeled specific binding member may have a plurality of attached label (e.g., 2 attached labels, 3 attached labels, 4 attached labels, etc.) or a plurality of fluorescent moieties (e.g., 2 moieties, 3 moieties, 4 moieties, etc.) provided the plurality is the same for each molecule of labeled specific binding member.

Specific binding agents of interest include antibody binding agents, proteins, peptides, haptens, nucleic acids, etc. The term “antibody binding agent” as used herein includes polyclonal or monoclonal antibodies or fragments that are sufficient to bind to an analyte of interest. The antibody fragments can be, for example, monomeric Fab fragments, monomeric Fab′ fragments, or dimeric F(ab)′2 fragments. Also within the scope of the term “antibody binding agent” are molecules produced by antibody engineering, such as single-chain antibody molecules (scFv) or humanized or chimeric antibodies produced from monoclonal antibodies by replacement of the constant regions of the heavy and light chains to produce chimeric antibodies or replacement of both the constant regions and the framework portions of the variable regions to produce humanized antibodies. Nucleic acid binding agents of interest are nucleic acids that specifically bind or specifically hybridize to biomarker nucleic acids in a cell. The length of these nucleic acids may vary, so long as it is sufficient for the oligonucleotide to serve as a specific binding agent, and in some instances ranges from 13 to 100 nt, such as 14 to 50 nt, e.g., 15 to 25 nt, including but not limited to, e.g., 15 nt, 16 nt, 17 nt, 18 nt, 19 nt, 20 nt, 21 nt, 22 nt, 23 nt, 24 nt, and 25 nt. The oligonucleotides that make up these nucleic acid binding agents may be DNA or RNA, or a synthetic analogue thereof, as desired.

As described above, the specific binding members described herein will generally be detectably labeled (i.e., have an attached detectable label, be bound by a detectable label, etc.). Therefore, in addition to a specific binding domain that specifically binds or specifically hybridizes to the biomarker of interest, the specific binding agent may further include or may be bound by or attached to a detectable label. Of interest as detectable labels are fluorescent dyes. Fluorescent dyes (fluorophores) can be selected from any of the many dyes suitable for use in imaging applications (e.g., fluorescent microscopy) and cytometry applications. A large number of dyes are commercially available from a variety of sources, such as, for example, Molecular Probes (Eugene, Oreg.) and Exciton (Dayton, Ohio). Examples of fluorophores of interest include, but are not limited to, 4-acetamido-4′-isothiocyanatostilbene-2,2′disulfonic acid; acridine and derivatives such as acridine, acridine orange, acridine yellow, acridine red, and acridine isothiocyanate; 5-(2′-aminoethyl)aminonaphthalene-1-sulfonic acid (EDANS); 4-amino-N-[3-vinylsulfonyl)phenyl]naphthalimide-3,5 disulfonate (Lucifer Yellow VS); N-(4-anilino-1-naphthyl)maleimide; anthranilamide; Brilliant Yellow; coumarin and derivatives such as coumarin, 7-amino-4-methylcoumarin (AMC, Coumarin 120), 7-amino-4-trifluoromethylcouluarin (Coumarin 151); cyanine and derivatives such as cyanosine, Cy3, Cy5, Cy5.5, and Cy7; 4′,6-diaminidino-2-phenylindole (DAPI); 5′, 5″-dibromopyrogallol-sulfonephthalein (Bromopyrogallol Red); 7-diethylamino-3-(4′-isothiocyanatophenyl)-4-methylcoumarin; diethylaminocoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansyl chloride); 4-(4′-dimethylaminophenylazo)benzoic acid (DABCYL); 4-dimethylaminophenylazophenyl-4′-isothiocyanate (DABITC); eosin and derivatives such as eosin and eosin isothiocyanate; erythrosin and derivatives such as erythrosin B and erythrosin isothiocyanate; ethidium; fluorescein and derivatives such as 5-carboxyfluorescein (FAM), 5-(4,6-dichlorotriazin-2-yl)aminofluorescein (DTAF), 2′7′-dimethoxy-4′5′-dichloro-6-carboxyfluorescein (JOE), fluorescein isothiocyanate (FITC), fluorescein chlorotriazinyl, naphthofluorescein, and QFITC (XRITC); fluorescamine; IR144; IR1446; Green Fluorescent Protein (GFP); Reef Coral Fluorescent Protein (RCFP); Lissamine™; Lissamine rhodamine, Lucifer yellow; Malachite Green isothiocyanate; 4-methylumbelliferone; ortho cresolphthalein; nitrotyrosine; pararosaniline; Nile Red; Oregon Green; Phenol Red; B-phycoerythrin; o-phthaldialdehyde; pyrene and derivatives such as pyrene, pyrene butyrate and succinimidyl 1-pyrene butyrate; Reactive Red 4 (Cibacron™ Brilliant Red 3B-A); rhodamine and derivatives such as 6-carboxy-X-rhodamine (ROX), 6-carboxyrhodamine (R6G), 4,7-dichlororhodamine lissamine, rhodamine B sulfonyl chloride, rhodamine (Rhod), rhodamine B, rhodamine 123, rhodamine X isothiocyanate, sulforhodamine B, sulforhodamine 101, sulfonyl chloride derivative of sulforhodamine 101 (Texas Red), N,N,N′,N′-tetramethyl-6-carboxyrhodamine (TAMRA), tetramethyl rhodamine, and tetramethyl rhodamine isothiocyanate (TRITC); riboflavin; rosolic acid and terbium chelate derivatives; xanthene; or combinations thereof. Other fluorophores or combinations thereof known to those skilled in the art may also be used, for example those available from Molecular Probes (Eugene, Oreg.) and Exciton (Dayton, Ohio).

The methods of the present disclosure generally include the use of a labeled binding member specific of PD-L1 (i.e., a labeled PD-L1 specific binding member) to label cells of the cell suspension thus generating a labeled cell suspension that may be cytometrically assayed. As described above, depending on the context, a labeled binding member specific for PD-L1 may specifically bind PD-L1 protein or may specifically bind PD-L1 transcript.

In some instances, a labeled binding member specific for PD-L1 protein may specifically bind PD-L1 protein expressed on the surface of a cell. Human PD-L1 protein is a 290 amino acid polypeptide having a signal peptide domain from residue 1 to about residue 18, an extracellular topological domain from about residue 19 to about residue 238, a transmembrane domain from about residue 239 to about residue 259, and a cytoplasmic topological domain from about residue 260 to 290. The primary isoform of human PD-L1 (programmed cell death 1 ligand 1 isoform a precursor NP_054862.1) has the following amino acid sequence: MRIFAVFIFMTYWHLLNAFTVTVPKDLYVVEYGSNMTIECKFPVEKQLDLAALIVYWEMEDK NI IQFVHGEEDLKVQHSSYRQRARLLKDQLSLGNAALQITDVKLQDAGVYRCMISYGGADY KRITVKVNAPYNKINQRILVVDPVTSEHELTCQAEGYPKAEVIWTSSDHQVLSGKTTTTNSK REEKLFNVTSTLRINTTTNEIFYCTFRRLDPEENHTAELVIPELPLAHPPNERTHLVILGAILLCL GVALTFIFRLRKGRMMDVKKCGIQDTNSKKQSDTHLEET (SEQ ID NO:1). Human PD-L1 also has a minor alternatively spliced isoform (programmed cell death 1 ligand 1 isoform b precursor NP_001254635.1) having the following amino acid sequence: MRIFAVFIFMTYWHLLNAPYNKINQRILVVDPVTSEHELTCQAEGYPKAEVIWTSSDHQVLSG KTTTTNSKREEKLFNVTSTLRINTTTNEIFYCTFRRLDPEENHTAELVIPELPLAHPPNERTHL VILGAILLCLGVALTFIFRLRKGRMMDVKKCGIQDTNSKKQSDTHLEET (SEQ ID NO:2).

Useful specific binding members specific for PD-L1 protein include but are not limited to antibodies, including but not limited to antibodies that bind one or both of the above human PD-L1 isoforms, the sequences of which are provided. In some instances, anti-PD-L1 (i.e., anti-CD274) antibodies that are directly conjugated to a fluorophore may find use in the described methods. Useful commercially available directly conjugated anti-PD-L1 antibodies include but are not limited to e.g., those listed in Table 1 below.

TABLE 1

| Commercial | | |
|---|---|---|
| Supplier | Antibody | Fluor. Conjugation |
| Acris Antibodies | Anti-human CD274/PDL1 | FITC, PE |
| GmbH | | |
| Acris Antibodies | Anti-mouse CD274/PDL1 | FITC |
| GmbH | | |
| Novus Biologicals | Goat Polyclonal Anti-Mouse B7- | Allophycocyanin, |
| | H1/PD-L1/CD274 Antibody | Fluorescein |
| Novus Biologicals | Mouse Monoclonal Anti-Human | Phycoerythrin, Alexa Fluor |
| | B7-H1/PD-L1/CD274 Antibody | 700, Alexa Fluor 647, Alexa |
| | | Fluor 594, Alexa Fluor 405, |
| | | Allophycocyanin, PerCP, |
| | | Phycoerythrin, Alexa Fluor |
| | | 488, Alexa Fluor 350, Alexa |
| | | Fluor 750 |
| Novus Biologicals | Rat Monoclonal Anti- | PerCP, Alexa Fluor 647, |
| | Human/Mouse B7-H1/PD- | Alexa Fluor 594, DyLight |
| | L1/CD274 Antibody | 488, Alexa Fluor 405, Alexa |
| | | Fluor 488, Alexa Fluor 647, |
| | | Alexa Fluor 700, DyLight |
| | | 755, DyLight 350, PE, |
| | | DyLight 680, |
| | | Allophycocyanin, DyLight |
| | | 405, DyLight 405LS |
| R&D Systems | Anti-Mouse B7-H1/PD-L1 | Allophycocyanin, |
| | Antibody | Fluorescein, Alexa Fluor |
| | | 594, Alexa Fluor 647 |
| R&D Systems | Anti-Human B7-H1/PD-L1 | Allophycocyanin, Alexa |
| | Antibody | Fluor 405, Alexa Fluor 488, |
| | | Alexa Fluor 594, Alexa Fluor |
| | | 647, Alexa Fluor 700, |
| | | Phycoerythrin, PerCP |
| Bio-Rad (Formerly | Rat anti-mouse CD274 Antibody | FITC, Alexa Fluor 488, |
| AbD Serotec) | | Alexa Fluor 647 |
| Bio-Rad (Formerly | Mouse anti-human CD274 | Alexa Fluor 488, Alexa Fluor |
| AbD Serotec) | Antibody | 647, FITC, RPE |
| GeneTex | Anti-human PD-L1 antibody | FITC, Phycoerythrin (PE) |
| GeneTex | Anti-mouse PD-L1 antibody | FITC |
| Tonbo | Anti-Mouse CD274 (PD-L1, B7- | PE |
| Biotechnologies | H1) (10F.9G2) | |
| LifeSpan BioSciences | Anti-human CD274/B7-H1/PD- | FITC, PE |
| | L1 Antibody | |
| LifeSpan BioSciences | Anti-human CD274/B7-H1/PD- | FITC, RPE |
| | L1 Antibody (clone MIH2) | |
| LifeSpan BioSciences | Anti-human CD274/B7-H1/PD- | FITC |
| | L1 Antibody (clone MIH6) | |
| LifeSpan BioSciences | Anti-human CD274/B7-H1/PD- | APC |
| | L1 Antibody (aa19-238, clone | |
| | 12K56) | |
| LifeSpan BioSciences | Anti-human CD274/B7-H1/PD- | PE |
| | L1 Antibody (clone 27A2) | |
| LifeSpan BioSciences | Anti-human CD274/B7-H1/PD- | FITC, RPE |
| | L1 Antibody (clone ANC6H1) | |
| LifeSpan BioSciences | Anti-mouse CD274/B7-H1/PD- | PE |
| | L1 Antibody (clone MIH5) | |
| LifeSpan BioSciences | Anti-mouse CD274/B7-H1/PD- | APC, FITC |
| | L1 Antibody (clone 10F.9G2) | |
| LifeSpan BioSciences | Anti-mouse CD274/B7-H1/PD- | APC, PE |
| | L1 Antibody (clone 29E.2A3) | |
| BioLegend | Anti-mouse CD274 (B7-H1, PD- | APC, Brilliant Violet 421, |
| | L1) Antibody | PE/DZL594, Brilliant Violet |
| | | 711, PE, PE/Cy7, Brilliant |
| | | Violet 605 |
| BioLegend | Anti-human CD274 (B7-H1, PD- | APC, Brilliant Violet 421, |
| | L1) Antibody | Brilliant Violet 711, Brilliant |
| | | Violet 605, APC, PE, |
| | | PE/Cy7, PE/DZL594, |
| | | PerCP/Cy5.5 |
| GenWay Biotech, Inc. | Anti-human CD274 Antibody | FITC |
| GenWay Biotech, Inc. | Anti-mouse CD274 Antibody | FITC |
| Abcam | Anti-human PD-L1 antibody | Phycoerythrin |
| | (MIH2) | |
| Abcam | Anti-human PD-L1 antibody (28-8) | Alexa Fluor 647 |
| Abcam | Anti-mouse PD-L1 antibody | Phycoerythrin |
| | (10F.9G2) | |
| BD Biosciences | Rat anti-mouse CD274 Antibody | PE, APC, BV711 |
| BD Biosciences | Mouse anti-human CD274 | APC, BB515, BV421, |
| | Antibody | BV650, BV786, FITC, PE, |
| | | PE-CF594, PE-Cy7 |
| Cell Signaling | Anti-human PD-L1 (E1L3N) XP | Alexa Fluor 488, Alexa Fluor |
| Technology | Rabbit mAb | 647, PE |

In some instances, an unconjugated anti-PD-L1 antibody may find use in the herein described methods, including but not limited to e.g., those unconjugated anti-PD-L1 antibodies available from commercial suppliers including e.g., those commercial suppliers listed above in Table 1. In some instances, an unconjugated anti-PD-L1 antibody may be conjugated prior to use including but not limited to e.g., where the unconjugated antibody is conjugated to a fluorophore.

Anti-PD-L1 protein specific binding members are not limited to antibodies and may also, in some instances, include e.g., anti-PD-L1 aptamers, anti-PD-L1 haptens, etc. Additionally, synthetic specific binding members specific for PD-L1 protein may also be derived from the PD-L1 binding portion of PD-1. For example, in some instances, a PD-L1 specific binding member may be rationally designed to include a PD-1-derived PD-L1 binding domain e.g., based on the binding interaction of PD-1 and PD-L1, e.g., as shown in RCSB Protein Data Bank (PDB) structure 4ZQK and described in Zak et al., (2015) Structure 23:2341-2348; the disclosure of which is incorporated herein by reference in its entirety.

In some instances, the methods described herein may make use of a labeled binding member specific for PD-L1 transcript. A labeled binding member specific for PD-L1 transcript may specifically bind PD-L1 mRNA expressed in a cell. Useful specific binding members specific for PD-L1 transcript include but are not limited to oligonucleotides having complementary sequence to all or a portion of the PD-L1 mRNA sequence. In some instances, a useful oligonucleotide probe may include a sequence complementary to a human PD-L1 mRNA transcript including but not limited to e.g.:

Homo sapiens CD274 molecule (CD274), transcript
variant 1, mRNA (NM_014143.3):
(SEQ ID NO: 3)
GGCGCAACGCTGAGCAGCTGGCGCGTCCCGCGCGGCCCCAGTTCTGCGCA
GCTTCCCGAGGCTCCGCACCAGCCGCGCTTCTGTCCGCCTGCAGGGCATT
CCAGAAAGATGAGGATATTTGCTGTCTTTATATTCATGACCTACTGGCAT
TTGCTGAACGCATTTACTGTCACGGTTCCCAAGGACCTATATGTGGTAGA
GTATGGTAGCAATATGACAATTGAATGCAAATTCCCAGTAGAAAAACAAT
TAGACCTGGCTGCACTAATTGTCTATTGGGAAATGGAGGATAAGAACATT
ATTCAATTTGTGCATGGAGAGGAAGACCTGAAGGTTCAGCATAGTAGCTA
CAGACAGAGGGCCCGGCTGTTGAAGGACCAGCTCTCCCTGGGAAATGCTG
CACTTCAGATCACAGATGTGAAATTGCAGGATGCAGGGGTGTACCGCTGC
ATGATCAGCTATGGTGGTGCCGACTACAAGCGAATTACTGTGAAAGTCAA
TGCCCCATACAACAAAATCAACCAAAGAATTTTGGTTGTGGATCCAGTCA
CCTCTGAACATGAACTGACATGTCAGGCTGAGGGCTACCCCAAGGCCGAA
GTCATCTGGACAAGCAGTGACCATCAAGTCCTGAGTGGTAAGACCACCAC
CACCAATTCCAAGAGAGAGGAGAAGCTTTTCAATGTGACCAGCACACTGA
GAATCAACACAACAACTAATGAGATTTTCTACTGCACTTTTAGGAGATTA
GATCCTGAGGAAAACCATACAGCTGAATTGGTCATCCCAGAACTACCTCT
GGCACATCCTCCAAATGAAAGGACTCACTTGGTAATTCTGGGAGCCATCT
TATTATGCCTTGGTGTAGCACTGACATTCATCTTCCGTTTAAGAAAAGGG
AGAATGATGGATGTGAAAAAATGTGGCATCCAAGATACAAACTCAAAGAA
GCAAAGTGATACACATTTGGAGGAGACGTAATCCAGCATTGGAACTTCTG
ATCTTCAAGCAGGGATTCTCAACCTGTGGTTTAGGGGTTCATCGGGGCTG
AGCGTGACAAGAGGAAGGAATGGGCCCGTGGGATGCAGGCAATGTGGGAC
TTAAAAGGCCCAAGCACTGAAAATGGAACCTGGCGAAAGCAGAGGAGGAG
AATGAAGAAAGATGGAGTCAAACAGGGAGCCTGGAGGGAGACCTTGATAC
TTTCAAATGCCTGAGGGGCTCATCGACGCCTGTGACAGGGAGAAAGGATA
CTTCTGAACAAGGAGCCTCCAAGCAAATCATCCATTGCTCATCCTAGGAA
GACGGGTTGAGAATCCCTAATTTGAGGGTCAGTTCCTGCAGAAGTGCCCT
TTGCCTCCACTCAATGCCTCAATTTGTTTTCTGCATGACTGAGAGTCTCA
GTGTTGGAACGGGACAGTATTTATGTATGAGTTTTTCCTATTTATTTTGA
GTCTGTGAGGTCTTCTTGTCATGTGAGTGTGGTTGTGAATGATTTCTTTT
GAAGATATATTGTAGTAGATGTTACAATTTTGTCGCCAAACTAAACTTGC
TGCTTAATGATTTGCTCACATCTAGTAAAACATGGAGTATTTGTAAGGTG
CTTGGTCTCCTCTATAACTACAAGTATACATTGGAAGCATAAAGATCAAA
CCGTTGGTTGCATAGGATGTCACCTTTATTTAACCCATTAATACTCTGGT
TGACCTAATCTTATTCTCAGACCTCAAGTGTCTGTGCAGTATCTGTTCCA
TTTAAATATCAGCTTTACAATTATGTGGTAGCCTACACACATAATCTCAT
TTCATCGCTGTAACCACCCTGTTGTGATAACCACTATTATTTTACCCATC
GTACAGCTGAGGAAGCAAACAGATTAAGTAACTTGCCCAAACCAGTAAAT
AGCAGACCTCAGACTGCCACCCACTGTCCTTTTATAATACAATTTACAGC
TATATTTTACTTTAAGCAATTCTTTTATTCAAAAACCATTTATTAAGTGC
CCTTGCAATATCAATCGCTGTGCCAGGCATTGAATCTACAGATGTGAGCA
AGACAAAGTACCTGTCCTCAAGGAGCTCATAGTATAATGAGGAGATTAAC
AAGAAAATGTATTATTACAATTTAGTCCAGTGTCATAGCATAAGGATGAT
GCGAGGGGAAAACCCGAGCAGTGTTGCCAAGAGGAGGAAATAGGCCAATG
TGGTCTGGGACGGTTGGATATACTTAAACATCTTAATAATCAGAGTAATT
TTCATTTACAAAGAGAGGTCGGTACTTAAAATAACCCTGAAAAATAACAC
TGGAATTCCTTTTCTAGCATTATATTTATTCCTGATTTGCCTTTGCCATA
TAATCTAATGCTTGTTTATATAGTGTCTGGTATTGTTTAACAGTTCTGTC
TTTTCTATTTAAATGCCACTAAATTTTAAATTCATACCTTTCCATGATTC
AAAATTCAAAAGATCCCATGGGAGATGGTTGGAAAATCTCCACTTCATCC
TCCAAGCCATTCAAGTTTCCTTTCCAGAAGCAACTGCTACTGCCTTTCAT
TCATATGTTCTTCTAAAGATAGTCTACATTTGGAAATGTATGTTAAAAGC
ACGTATTTTTAAAATTTTTTTCCTAAATAGTAACACATTGTATGTCTGCT
GTGTACTTTGCTATTTTTATTTATTTTAGTGTTTCTTATATAGCAGATGG
AATGAATTTGAAGTTCCCAGGGCTGAGGATCCATGCCTTCTTTGTTTCTA
AGTTATCTTTCCCATAGCTTTTCATTATCTTTCATATGATCCAGTATATG
TTAAATATGTCCTACATATACATTTAGACAACCACCATTTGTTAAGTATT
TGCTCTAGGACAGAGTTTGGATTTGTTTATGTTTGCTCAAAAGGAGACCC
ATGGGCTCTCCAGGGTGCACTGAGTCAATCTAGTCCTAAAAAGCAATCTT
ATTATTAACTCTGTATGACAGAATCATGTCTGGAACTTTTGTTTTCTGCT
TTCTGTCAAGTATAAACTTCACTTTGATGCTGTACTTGCAAAATCACATT
TTCTTTCTGGAAATTCCGGCAGTGTACCTTGACTGCTAGCTACCCTGTGC
CAGAAAAGCCTCATTCGTTGTGCTTGAACCCTTGAATGCCACCAGCTGTC
ATCACTACACAGCCCTCCTAAGAGGCTTCCTGGAGGTTTCGAGATTCAGA
TGCCCTGGGAGATCCCAGAGTTTCCTTTCCCTCTTGGCCATATTCTGGTG
TCAATGACAAGGAGTACCTTGGCTTTGCCACATGTCAAGGCTGAAGAAAC
AGTGTCTCCAACAGAGCTCCTTGTGTTATCTGTTTGTACATGTGCATTTG
TACAGTAATTGGTGTGACAGTGTTCTTTGTGTGAATTACAGGCAAGAATT
GTGGCTGAGCAAGGCACATAGTCTACTCAGTCTATTCCTAAGTCCTAACT
CCTCCTTGTGGTGTTGGATTTGTAAGGCACTTTATCCCTTTTGTCTCATG
TTTCATCGTAAATGGCATAGGCAGAGATGATACCTAATTCTGCATTTGAT
TGTCACTTTTTGTACCTGCATTAATTTAATAAAATATTCTTATTTATTTT
GTTACTTGGTACACCAGCATGTCCATTTTCTTGTTTATTTTGTGTTTAAT
AAAATGTTCAGTTTAACATCCCAGTGGAGAAAGTTAAAAAA
or
Homo sapiens CD274 molecule (CD274), transcript
variant 2, mRNA (NM_001267706.1):
(SEQ ID NO: 4)
GGCGCAACGCTGAGCAGCTGGCGCGTCCCGCGCGGCCCCAGTTCTGCGCA
GCTTCCCGAGGCTCCGCACCAGCCGCGCTTCTGTCCGCCTGCAGGGCATT
CCAGAAAGATGAGGATATTTGCTGTCTTTATATTCATGACCTACTGGCAT
TTGCTGAACGCCCCATACAACAAAATCAACCAAAGAATTTTGGTTGTGGA
TCCAGTCACCTCTGAACATGAACTGACATGTCAGGCTGAGGGCTACCCCA
AGGCCGAAGTCATCTGGACAAGCAGTGACCATCAAGTCCTGAGTGGTAAG
ACCACCACCACCAATTCCAAGAGAGAGGAGAAGCTTTTCAATGTGACCAG
CACACTGAGAATCAACACAACAACTAATGAGATTTTCTACTGCACTTTTA
GGAGATTAGATCCTGAGGAAAACCATACAGCTGAATTGGTCATCCCAGAA
CTACCTCTGGCACATCCTCCAAATGAAAGGACTCACTTGGTAATTCTGGG
AGCCATCTTATTATGCCTTGGTGTAGCACTGACATTCATCTTCCGTTTAA
GAAAAGGGAGAATGATGGATGTGAAAAAATGTGGCATCCAAGATACAAAC
TCAAAGAAGCAAAGTGATACACATTTGGAGGAGACGTAATCCAGCATTGG
AACTTCTGATCTTCAAGCAGGGATTCTCAACCTGTGGTTTAGGGGTTCAT
CGGGGCTGAGCGTGACAAGAGGAAGGAATGGGCCCGTGGGATGCAGGCAA
TGTGGGACTTAAAAGGCCCAAGCACTGAAAATGGAACCTGGCGAAAGCAG
AGGAGGAGAATGAAGAAAGATGGAGTCAAACAGGGAGCCTGGAGGGAGAC
CTTGATACTTTCAAATGCCTGAGGGGCTCATCGACGCCTGTGACAGGGAG
AAAGGATACTTCTGAACAAGGAGCCTCCAAGCAAATCATCCATTGCTCAT
CCTAGGAAGACGGGTTGAGAATCCCTAATTTGAGGGTCAGTTCCTGCAGA
AGTGCCCTTTGCCTCCACTCAATGCCTCAATTTGTTTTCTGCATGACTGA
GAGTCTCAGTGTTGGAACGGGACAGTATTTATGTATGAGTTTTTCCTATT
TATTTTGAGTCTGTGAGGTCTTCTTGTCATGTGAGTGTGGTTGTGAATGA
TTTCTTTTGAAGATATATTGTAGTAGATGTTACAATTTTGTCGCCAAACT
AAACTTGCTGCTTAATGATTTGCTCACATCTAGTAAAACATGGAGTATTT
GTAAGGTGCTTGGTCTCCTCTATAACTACAAGTATACATTGGAAGCATAA
AGATCAAACCGTTGGTTGCATAGGATGTCACCTTTATTTAACCCATTAAT
ACTCTGGTTGACCTAATCTTATTCTCAGACCTCAAGTGTCTGTGCAGTAT
CTGTTCCATTTAAATATCAGCTTTACAATTATGTGGTAGCCTACACACAT
AATCTCATTTCATCGCTGTAACCACCCTGTTGTGATAACCACTATTATTT
TACCCATCGTACAGCTGAGGAAGCAAACAGATTAAGTAACTTGCCCAAAC
CAGTAAATAGCAGACCTCAGACTGCCACCCACTGTCCTTTTATAATACAA
TTTACAGCTATATTTTACTTTAAGCAATTCTTTTATTCAAAAACCATTTA
TTAAGTGCCCTTGCAATATCAATCGCTGTGCCAGGCATTGAATCTACAGA
TGTGAGCAAGACAAAGTACCTGTCCTCAAGGAGCTCATAGTATAATGAGG
AGATTAACAAGAAAATGTATTATTACAATTTAGTCCAGTGTCATAGCATA
AGGATGATGCGAGGGGAAAACCCGAGCAGTGTTGCCAAGAGGAGGAAATA
GGCCAATGTGGTCTGGGACGGTTGGATATACTTAAACATCTTAATAATCA
GAGTAATTTTCATTTACAAAGAGAGGTCGGTACTTAAAATAACCCTGAAA
AATAACACTGGAATTCCTTTTCTAGCATTATATTTATTCCTGATTTGCCT
TTGCCATATAATCTAATGCTTGTTTATATAGTGTCTGGTATTGTTTAACA
GTTCTGTCTTTTCTATTTAAATGCCACTAAATTTTAAATTCATACCTTTC
CATGATTCAAAATTCAAAAGATCCCATGGGAGATGGTTGGAAAATCTCCA
CTTCATCCTCCAAGCCATTCAAGTTTCCTTTCCAGAAGCAACTGCTACTG
CCTTTCATTCATATGTTCTTCTAAAGATAGTCTACATTTGGAAATGTATG
TTAAAAGCACGTATTTTTAAAATTTTTTTCCTAAATAGTAACACATTGTA
TGTCTGCTGTGTACTTTGCTATTTTTATTTATTTTAGTGTTTCTTATATA
GCAGATGGAATGAATTTGAAGTTCCCAGGGCTGAGGATCCATGCCTTCTT
TGTTTCTAAGTTATCTTTCCCATAGCTTTTCATTATCTTTCATATGATCC
AGTATATGTTAAATATGTCCTACATATACATTTAGACAACCACCATTTGT
TAAGTATTTGCTCTAGGACAGAGTTTGGATTTGTTTATGTTTGCTCAAAA
GGAGACCCATGGGCTCTCCAGGGTGCACTGAGTCAATCTAGTCCTAAAAA
GCAATCTTATTATTAACTCTGTATGACAGAATCATGTCTGGAACTTTTGT
TTTCTGCTTTCTGTCAAGTATAAACTTCACTTTGATGCTGTACTTGCAAA
ATCACATTTTCTTTCTGGAAATTCCGGCAGTGTACCTTGACTGCTAGCTA
CCCTGTGCCAGAAAAGCCTCATTCGTTGTGCTTGAACCCTTGAATGCCAC
CAGCTGTCATCACTACACAGCCCTCCTAAGAGGCTTCCTGGAGGTTTCGA
GATTCAGATGCCCTGGGAGATCCCAGAGTTTCCTTTCCCTCTTGGCCATA
TTCTGGTGTCAATGACAAGGAGTACCTTGGCTTTGCCACATGTCAAGGCT
GAAGAAACAGTGTCTCCAACAGAGCTCCTTGTGTTATCTGTTTGTACATG
TGCATTTGTACAGTAATTGGTGTGACAGTGTTCTTTGTGTGAATTACAGG
CAAGAATTGTGGCTGAGCAAGGCACATAGTCTACTCAGTCTATTCCTAAG
TCCTAACTCCTCCTTGTGGTGTTGGATTTGTAAGGCACTTTATCCCTTTT
GTCTCATGTTTCATCGTAAATGGCATAGGCAGAGATGATACCTAATTCTG
CATTTGATTGTCACTTTTTGTACCTGCATTAATTTAATAAAATATTCTTA
TTTATTTTGTTACTTGGTACACCAGCATGTCCATTTTCTTGTTTATTTTG
TGTTTAATAAAATGTTCAGTTTAACATCCCAGTGGAGAAAGTTAAAAAA.

Useful oligonucleotide probes may be DNA or RNA based and may include but are not limited to those probes used in in situ hybridization, including e.g., DNA in situ hybridization probes, RNA in situ hybridization probes (e.g., riboprobes), as well as anti-sense probes having one or more synthetic components including e.g., one or more synthetic nucleoside bases (such as e.g., a locked nucleic acid (LNA) and the like). Such probes may vary in length, ranging in some instances from 13 to 100 nt, such as 14 to 50 nt, e.g., 15 to 25 nt, etc. Oligonucleotide probes may be directly conjugated with a fluorophore, including e.g., those fluorophores described herein. In some instances, oligonucleotide probes may be conjugated with a moiety that allows for binding of a label once the oligonucleotide is hybridized. For example, an oligonucleotide may be conjugated to one or more biotin molecules allowing the oligonucleotide to be labeled after hybridization e.g., by introducing fluorescently labeled streptavidin.

Reagents useful in the herein described methods may also include labeled specific binding members specific for immune cells. Such specific binding members may allow for the identification of immune cells within a cell population of the instant disclosure and/or identification of a cell as not being an immune cell as described above.

Any convenient labeled specific binding member for an immune cell may find use in the herein described methods including but not limited to e.g., antibodies specific for individual immune cell markers including but not limited to e.g., an anti-CD114 antibody, an anti-CD117 antibody, an anti-CD11a antibody, an anti-CD11b antibody, an anti-CD14 antibody, an anti-CD15 antibody, an anti-CD16 antibody, an anti-CD182 antibody, an anti-CD19 antibody, an anti-CD20 antibody, an anti-CD22 antibody, an anti-CD24 antibody, an anti-CD25 antibody, an anti-CD3 antibody, an anti-CD30 antibody, an anti-CD31 antibody, an anti-CD34 antibody, an anti-CD38 antibody, an anti-CD4 antibody, an anti-CD45 antibody, an anti-CD56 antibody, an anti-CD61 antibody, an anti-CD8 antibody, an anti-CD91 antibody, an anti-Foxp3 antibody, and the like.

In some instances, useful specific binding members for an immune cell may include anti-CD45 antibodies. Non-limiting examples of useful anti-CD45 antibodies include but are not limited to e.g., CD45-PC7 Conjugated Antibody (Beckman Coulter, Inc.); CD45/PTPRC Antibody, Mouse Mab (Sino Biological, Inc.); Anti-CD45 antibody [EP322Y] (Abcam); CD45/LCA Monoclonal Antibody (LifeSpan BioSciences); APC Anti-Human CD45 (H130) (Tonbo Biosciences); CD45 Allophycocyanin Antibody (Rockland Immunochemicals, Inc.); CD45 Antibody (FITC/APC) (BioLegend); CD45 Antibody (2B11) [Allophycocyanin] (Novus Biologicals); CD45 Antibody (2B11) [PE] (Novus Biologicals); Anti-CD45, clone F10-89.4, PE Conjugate (MilliporeSigma); Anti-CD45 (human) Antibody, AlexaFluor®488, clone H130: MABF323 (MilliporeSigma); Anti CD45 Antibody (PE-Vio770) (Miltenyi Biotec); and the like.

As described above, e.g., regarding the detection of DNA content, the herein described methods may include detection of DNA using one or more DNA labeling reagents. Various DNA labeling reagents may find use in the herein described methods including but not limited to: Hoechst 33342 (2′-(4-Ethoxyphenyl)-5-(4-methyl-1-piperazinyl)-1H,1′H-2,5′-bibenzimidazole trihydrochloride) and Hoechst 33258 (4-[6-(4-Methyl-1-piperazinyl)-1′,3′-dihydro-1H,2′H-2,5′-bibenzimidazol-2′-ylidene]-2,5-cyclohexadien-1-one trihydrochloride) and others of the Hoechst series; SYTO 40, SYTO 11, 12, 13, 14, 15, 16, 20, 21, 22, 23, 24, 25 (green); SYTO 17, 59 (red), DAPI, DRAQS™ (an anthraquinone dye with high affinity for double stranded DNA), YOYO-1, propidium iodide, YO-PRO-3, TO-PRO-3, YOYO-3 and TOTO-3, SYTOX Green, SYTOX, methyl green, acridine homodimer, 7-aminoactinomycin D, 9-amino-6-chloro-2-methoxyactridine.

The above-described markers include intracellular markers. As used herein, the term “intracellular markers” refers to components of the cell that are within the cell beyond the outer surface of the plasma membrane. Such components may be or may be within any interior component of the cell including but not limited to the inner surface of the plasma membrane, the cytoplasm, the nucleus, mitochondria, endoplasmic reticulum, etc. As such, labeling or detection of intracellular markers requires transport of a specific label or specific binding agent of the intracellular marker across at least the outer surface of the plasma membrane. In some instances, a label or specific binding agent for an intracellular marker may be membrane permeable thus not requiring modulation of membrane permeability for labeling of the intracellular marker. In some embodiments, a label or specific binding agent for an intracellular marker may be membrane impermeable thus requiring modulation of membrane permeability for labeling of the intracellular marker, including, e.g., preparation and or treatment of the cells with one or more permeabilizing reagents as described herein.

The above-described markers include cell surface markers. As used herein, the term “cell surface markers” refers to components of the cell that are at least exposed, partially or completely, on the outer surface of the plasma membrane of cell and thus may be accessed without modulating cell permeability, e.g., without the use of one or more permeabilizing reagents as described herein. In some instances, cell surface markers include components of the cell that have a portion exposed on the outer surface of the cell membrane but also contain an intracellular portion and/or a transmembrane portion.

As described herein and as will be readily apparent to one or ordinary skill in the art, any combination of the agents and labels described herein may be employed in the methods described provided the combination is appropriate and the components do not physically or optically interfere. For example, where alterations or substitutions of particular labels can and/or should be employed in order to allow for the combination of two or more desired components is within the skill of the ordinary artisan. As a non-limiting example, where a particular fluorescent label of a biomarker interferes optically (e.g., has an overlapping emission spectra) with a desired DNA labeling agent of a particular emission wavelength, the fluorescent label of the biomarker may be substituted with a different fluorescent label having no or less emission spectra overlap with the desired DNA labeling agent.

Methods of Treating

As summarized above, the present disclosure includes methods of treating a subject for a malignant bladder-associated neoplasia. The terms “subject,” “individual,” “host,” and “patient,” are used interchangeably herein and refer to any mammalian subject for whom diagnosis, treatment, or therapy is desired, particularly humans.

Aspects of the subject methods generally include identifying a malignant bladder-associated neoplasia in a subject and treating the subject by administering to the subject an anti-PD-1/PD-L1 immunotherapy. Accordingly, in some instances, a subject may be identified as having an anti-PD-1/PD-L1 immunotherapy responsive malignant bladder-associated neoplasia. Such identification may be performed according to any of the methods described herein, including methods where per cell PD-L1 expression is detected. In some instances, such methods may include cytometrically detecting whether an immune cell that expresses PD-L1, e.g., above a predetermined threshold, is present in a bladder-associated sample from the subject. In some instances, such methods may include detecting a PD-L1-aneuploid-to-PD-L1-epithelial ratio of a sample that is above a predetermined threshold. In some instances, the ratio may be employed to differentiate between high grade (HG) and low grade (LG) transitional cell carcinoma of the bladder (BC).

In some instances, such detecting will further include cytometrically assaying the labeled cell suspension to quantify one or more of the percentage of cells that are immune cells, per cell DNA index, epithelial cell aneuploidy, the percentage of cells that are epithelial cells, the percentage of cells that are PD-L1 positive epithelial cells, the percentage of epithelial cells that are aneuploid, the percent of PD-L1 positive epithelial cells that are aneuploid, the mean fluorescence of PD-L1 positive epithelial cells, the mean fluorescence of PD-L1 negative epithelial cells, the mean fluorescence of PD-L1 positive aneuploid cells, the mean fluorescence of PD-L1 negative aneuploid cells, or a combination thereof, to derive a further cytometric parameter. Identifying that the malignant bladder-associated neoplasia is present may include when an immune cell that expresses PD-L1 is present and the further cytometric parameter is above a threshold. Identifying that the malignant bladder-associated neoplasia is absent may include when an immune cell that expresses PD-L1 is absent and/or the further cytometric parameter is below the threshold. Identifying that a malignant bladder-associated neoplasia is present may include generating a PD-L1-aneuploid-to-PD-L1-epithelial ratio and identifying that the ratio is above a threshold. Identifying that a malignant bladder-associated neoplasia is absent may include generating a PD-L1-aneuploid-to-PD-L1-epithelial ratio and identifying that the ratio is below a threshold. In some instances, a combination of such analyses may be combined to identify whether a malignant bladder-associated neoplasia is present or absent. In some instances, the ratio may be employed to differentiate between high grade (HG) and low grade (LG) transitional cell carcinoma of the bladder (BC).

Subjects tested and/or treated according to the herein described methods include subjects having a bladder-associated neoplasia, subjects having a malignant bladder-associated neoplasia, subjects suspected of having a bladder-associated neoplasia, subjects suspected of having a malignant bladder-associated neoplasia, subjects at an increased risk of having a bladder-associated neoplasia, subjects at an increased risk of having a malignant bladder-associated neoplasia, subject that have been previously treated for a bladder-associated neoplasia, subject that have been previously treated for a malignant bladder-associated neoplasia, subjects that have not been previously treated for a bladder-associated neoplasia, and the like.

Subjects treated in the present methods may or may not have been previously treated. For example, in some instances, the subject has been previously treated with chemotherapy. In some instances, the subject has not been previously treated with chemotherapy. In some instances, the subject has been previously treated with radiation therapy. In some instances, the subject has not been previously treated with radiation therapy. In some instances, the subject has been previously treated with immunotherapy. In some instances, the subject has not been previously treated with immunotherapy.

Accordingly, in some instances, a subject treated in the present methods may have previously undergone treatment, where e.g., the previous treatment was not successful, and/or the subject has a new, recurrent, or re-growing neoplasia. Such previous treatments may include but are not limited to e.g., chemotherapy with or without neoadjuvant and/or adjuvant and immunotherapy, intravesical therapy (including e.g., BCG, Mitomycin, Gemcitabine, or the like), and the like.

In some instances, subjects treated according to the herein described methods may be subjects having a neoplasia suspected of or showing symptoms of immune evasion. Various indicators may suggest immune evasion by a neoplasia including but not limited to e.g., tumor growth or progression, immunosuppression, unresponsiveness to immunotherapy, and the like.

Various factors and events present in the tumor microenvironment may be directly indicative of immune evasion or tumor progression, which indirectly indicates immune evasion, including but not limited to e.g., presence of activated T cells in the absence of appropriate costimulation, tumor cell expression of T cell-inhibitory molecules (e.g., HLA-G, HLA-E, etc.), tumor antigen loss, downregulation of MHC molecules, regulatory T cells (Tregs) (e.g., CD4+CD25+ Tregs, CD4+CD25+ FoxP3+, etc.), presence of CD1d-restricted T cells, immunosuppressive factors and tumor-derived cytokines (e.g., transforming growth factor (TGF)-β, tumor necrosis factor (TNF)-α, VEGF, IL-1, IL-1β, IL-6, IL-8, IL-10, GM-CSF, type I IFNs, gangliosides, receptor-binding cancer-associated surface antigen (RCAS1), etc.), presence of immunosuppressive myeloid cell populations (immature myeloid cell populations e.g., those expressing iNOS (also known as NOS2) or arginase 1 (ARG1), myeloid-derived suppressor cells (MDSCs), modulated dendritic cells (DCs), alternatively-activated M1 and M2 macrophages, CD11b+Gr1+ MDSCs, etc.), TCR ζ-chain downregulation, upregulation of immunosuppressive enzymes (e.g., indoleamine 2,3-dioxygenase (IDO), arginase, inhibitor of nuclear factor kappa-B kinase (IKK)2, etc.), and the like. In some instances, particular tumor characteristics may also be indicative of immune evasion, including but not limited to e.g., tumor resistance to cytotoxic pathways (e.g., as seen in tumors with FAS mutations), mutations in the gene encoding the TRAIL receptor death receptor 5 (DR5), overexpression of the anti-apoptotic molecules (e.g., FLIP, BCL-XL, etc.), and the like.

In some instances, a subject with a bladder-associated neoplasia may be tested to determine whether the bladder-associated neoplasia is malignant and, if so, the subject may be subsequently treated with an appropriate therapy, such as e.g., with an anti-PD-1/PD-L1 immunotherapy. Any bladder-associated sample may be assayed to assess the likelihood that the subject's neoplasia is anti-PD-1/PD-L1 immunotherapy responsive, including whether or not the subject's neoplasia has previously shown indicators of metastasis and/or immune evasion.

Neoplasia therapies that may be administered to a subject before, during or after a subject is assessed will vary depending on numerous factors including e.g., the type of neoplasia, the subject's medical history, general state of health and/or any co-morbidities, and the like. Useful neoplasia therapies include but are not limited to e.g., radiation therapy, chemotherapy, immunotherapy, and the like. Neoplasia therapies, such as but not limited to radiation therapy, chemotherapy, immunotherapy, and the like, may be administered locally or systemically.

In some instances, a subject may be assessed before a course of therapy is begun including but not limited to e.g., immunotherapy. For example, in some instances, a medical professional may assay a subject to whether the subject has a malignant bladder-associated neoplasia prior to administering an anti-PD-1/PD-L1 immunotherapy and the medical professional may administer the therapy only if the subject's neoplasia is identified as likely to be a malignant bladder-associated neoplasia (i.e., a malignant bladder-associated neoplasia is likely to be present).

The amount of time before starting a course of treatment that a subject may be assessed may vary and may range from 1 day or less to a month or more including but not limited to e.g., 1 day, 2 days, 3 days, 4 days, 5 days, 6 days, 1 week, 2 weeks, 3 weeks, 1 month, etc. In some instances, a course of treatment may be begun the same day that an assessment for malignant bladder-associated neoplasia is performed.

In some instances, a subject may be assessed after a course of treatment has already been administered. For example, in some instances, a sample from a subject may be assayed after treatment has begun or after a failed course of therapy, including but not limited to e.g., chemotherapy, radiation therapy, immunotherapy, etc. In some instances, if the assessment identifies the presences of malignant bladder-associated neoplasia then an anti-PD-1/PD-L1 immunotherapy may be attempted, including where such an attempt is the first attempt or a subsequent attempt, such as e.g., a second attempt. In some instances, if the assessment does not identify or identifies the absence of malignant bladder-associated neoplasia then the medical professional may not attempt anti-PD-1/PD-L1 immunotherapy and no therapy or some other course of therapy (e.g., non-PD-L1 immunotherapy, chemotherapy, radiation therapy, etc.) may be warranted.

The amount of time after a course of treatment has ended that a subject may be assessed to determine whether malignant bladder-associated neoplasia is present may vary and may range from 1 day or less to a month or more including but not limited to e.g., 1 day, 2 days, 3 days, 4 days, 5 days, 6 days, 1 week, 2 weeks, 3 weeks, 1 month, etc. In some instances, an assessment is performed the same day on which the course of therapy is ended. In some instances, an assessment may be performed during a follow-up assessment, such as e.g., a long-term follow-up assessment, of a subject. The length of time after a course of treatment at which point long-term follow-up is performed will vary and may range from 3 months or less to 10 years or more including but not limited to e.g., 3 months, 4 months, 5 months, 6 months, 7 months, 8 months, 9 months, 10 months, 11 months, one year, 1.5 years, 2 years, 3 years, 4 years, 5 years, 6 years, 7 years, 8 years, 9 years, 10 years, etc.

In some instances, an assessment may be performed during a course of therapy targeted to treat the subject for the neoplasia. For example, a course of therapy to treat a subject for a neoplasia may be begun and during the course of therapy one or more assessments may be performed, e.g., to monitor the therapy. Such assessments may be performed for a variety of reasons including but not limited to e.g., the assess whether to continue the therapy, to assess whether to alter the course of therapy (e.g., change the drug being administered, change the dose of drug being administered, change the frequency of administration, etc.). For example, based on the outcome of an assessment during a course of therapy the subject may be switched to a different therapy or the subject's dose of may be increased or decreased or the frequency of administering the therapy may be increased or decreased or the therapy may be terminated.

Assessments made during a course of treatment may also be referred to herein as monitoring. For example, in some instances, a subject undergoing radiation therapy may be monitored and, if a malignant bladder-associated neoplasia is identified then the original course of radiation therapy may be altered or terminated and a new course of therapy, e.g., anti-PD-1/PD-L1 immunotherapy, may be initiated, in conjunction with or instead of the radiation therapy. In some instances, a subject undergoing chemotherapy may be monitored and, if a malignant bladder-associated neoplasia is identified then the original course of chemotherapy may be altered or terminated and a new course of therapy, e.g., anti-PD-1/PD-L1 immunotherapy, may be initiated, in conjunction with or instead of the chemotherapy. In some instances, a subject undergoing immunotherapy may be monitored and, if a malignant bladder-associated neoplasia is identified then the original course of immunotherapy may be altered or terminated and a new course of therapy, e.g., anti-PD-1/PD-L1 immunotherapy, may be initiated, in conjunction with or instead of the immunotherapy.

Where indicated any anti-PD-1/PD-L1 immunotherapy may find use in the subject methods including but not limited to e.g., those therapies that include administering to a subject an effective amount of one or more anti-PD-1/PD-L1 therapeutic antagonists where such antagonists include but are not limited to e.g., OPDIVO® (nivolumab), KEYTRUDA® (pembrolizumab), Tecentriq™ (atezolizumab), durvalumab (MED14736), avelumab (MSB0010718C), BMS-936559 (MDX-1105), CA-170, BMS-202, BMS-8, BMS-37, BMS-242 and the like.

Nivolumab (OPDIVO®) is a humanized IgG4 anti-PD-1 monoclonal antibody used to treat cancer. Pembrolizumab (KEYTRUDA®), formerly known as MK-3475, lambrolizumab, etc., is a humanized antibody used in cancer immunotherapy targeting the PD-1 receptor. Atezolizumab (Tecentriq™) is a fully humanized, engineered monoclonal antibody of IgG1 isotype against the PD-L1 protein. Durvalumab (Medlmmune) is a therapeutic monoclonal antibody that targets PD-L1. Avelumab (also known as MSB0010718C; Merck KGaA, Darmstadt, Germany & Pfizer) is a fully human monoclonal PD-L1 antibody of isotype IgG1. BMS-936559 (also known as MDX-1105; Bristol-Myers Squibb) is a blocking antibody that has been shown to bind to PD-L1 and prevent its binding to PD-1 (see e.g., U.S. NIH Clinical Trial No. NCT00729664). CA-170 (Curis, Inc.) is a small molecule PD-L1 antagonist. BMS-202, BMS-8, BMS-37, BMS-242 are small molecule PD-1/PD-L1 complex antagonists that bind PD-1 (see e.g., Kaz et al., (2016) Oncotarget 7(21); the disclosure of which is incorporated herein by reference in its entirety).

Anti-PD-L1 antagonists, including e.g., antibodies, useful in the methods described herein include but are not limited to e.g., those described in U.S. Pat. Nos. 7,722,868; 7,794,710; 7,892,540; 7,943,743; 8,168,179; 8,217,149; 8,354,509; 8,383,796; 8,460,927; 8,552,154; 8,741,295; 8,747,833; 8,779,108; 8,952,136; 8,981,063; 9,045,545; 9,102,725; 9,109,034; 9,175,082; 9,212,224; 9,273,135 and 9,402,888; the disclosures of which are incorporated herein by reference in their entirety.

Anti-PD-1 antagonists, including e.g., antibodies, useful in the methods described herein include but are not limited to e.g., those described in U.S. Pat. Nos. 6,808,710; 7,029,674; 7,101,550; 7,488,802; 7,521,051; 8,008,449; 8,088,905; 8,168,757; 8,460,886; 8,709,416; 8,951,518; 8,952,136; 8,993,731; 9,067,998; 9,084,776; 9,102,725; 9,102,727; 9,102,728; 9,109,034; 9,181,342; 9,205,148; 9,217,034; 9,220,776; 9,308,253; 9,358,289; 9,387,247 and 9,402,899; the disclosures of which are incorporated herein by reference in their entirety.

Compositions that include one or more of the subject anti-PD-1/PD-L1 antagonists may be administered once per day, a few or several times per day, or even multiple times per day, depending upon, among other things, the indication being treated and the judgment of the prescribing physician.

Methods of administration may be chosen depending on the condition being treated and the pharmaceutical composition being administered, such as e.g., the anti-PD-1/PD-L1 pharmaceutical composition being administered. Administration of the subject agent(s) can be done in a variety of ways, including, but not limited to, local administration of the agent, including e.g., subcutaneously, intravenously, intraperitoneally, intramuscularly, and possibly direct injection to specified organs or tumors, although systemic administration may also be used. Administration of the pharmaceutical compositions may be through a single route or concurrently by several routes.

In some instances, direct delivery to the urinary tract, or bladder specifically, may be employed, including but not limited to e.g., where such delivery methods involve intravesical delivery. Various methods and systems for direct urinary tract delivery may be employed, and/or adapted for use, in the herein described methods, including but not limited to e.g., those described in U.S. Pat. Nos. 10,029,012; 9,962,373; 9,943,576; 9,636,488; 9,295,666; 8,648,108; 7,320,963; 6,524,608; 6,207,180; 6,183,461; 6,171,298; and 6,039,967; the disclosures of which are incorporated herein by reference in their entirety.

By “effective amount” is meant an amount sufficient to have a therapeutic effect. An effective amount that will treat a neoplasia will modulate the symptoms and/or the size of the neoplasia typically by at least about 1%, including but not limited to e.g., at least about 10%; at least about 20%; at least about 30%; at least about 50%. Such will result in, e.g., statistically significant and quantifiable changes in the numbers of cells being affected. This may be a decrease in the size of the primary tumor, a decrease in the numbers of micrometastases in distant organs, a decrease in recurrent metastatic disease, etc.

The methods of the present disclosure may, in some embodiments, provide for certain advantages such as but not limited to e.g., improved detection of a particular cell type, improved treatment decisions and/or improved treatment outcomes, and the like. Improved detection may include detection with improved specificity, detection with improved sensitivity, and detection with improved sensitivity and specificity. Advantages of improved detection may also include, in some embodiments, detection that is more efficient, more convenient, and/or more cost-effective. Improved convenience may include but is not limited to e.g., where the sample analyzed in the assay is more conveniently acquired as compared to the acquisition of a sample using in a comparable assay (e.g., a urine sample may be more conveniently acquired as compared to a bladder biopsy sample). Improved treatment decisions include not only those decisions resulting in improved treatment outcomes, but also, in some instances, improved decisions as to whether or not to treat a subject, decisions preventing needless administration of an agent, treatments that are more cost-effective, efficient, and/or convenient, and the like. Other advantages, in some embodiments, may include but are not limited to reducing the number and/or different types of samples that need to be collected from a subject and/or analyzed, improved adherence with a specified treatment regimen, improved patient confidence in a prescribed treatment regimen, and the like.

In some instances, methods of the present disclosure provide the advantage of a test that can differentiate between benign and malignant microscopic hematuria to achieve one or more of the following benefits: decreased patient anxiety, reduced invasive testing (cystoscopy), decreased requirements for imaging and/or the intensity thereof, reduced repeat testing, and the like. In some embodiments, the methods of the present disclosure reduce the frequency and cost of follow up. In some embodiments, the methods of the present disclosure predict response to immunotherapy in superficial high-grade disease. In some embodiments, the methods of the present disclosure predict response to immunotherapy and/or chemotherapy in muscle invasive bladder cancer.

In some instances, the herein described methods of detecting anti-PD-1/PD-L1 immunotherapy responsive cells in a subject may serve to limit the administration of an anti-PD-1/PD-L1 immunotherapy to a subject having an immune-related disorder or a subject that is at increased risk of developing an immune-related disorder. By virtue of PD1 being naturally expressed on immune cells, PD-1/PD-L1 targeted therapies can negatively impact immune cells of a subject. In some instances, the methods of the present disclosure may be used to screen subjects prior to anti-PD-1/PD-L1 immunotherapy, including e.g., those subjects most likely to be negatively impacted by anti-PD-1/PD-L1 immunotherapy or have adverse events due to anti-PD-1/PD-L1 immunotherapy, including e.g., those subjects with immune-related disorders.

Non-limiting examples of immune-related disorders include but are not limited to e.g., autoimmune disorders such as e.g., Acute Disseminated Encephalomyelitis (ADEM), Acute necrotizing hemorrhagic leukoencephalitis, Addison's disease, Agammaglobulinemia, Alopecia areata, Amyloidosis, Ankylosing spondylitis, Anti-GBM/Anti-TBM nephritis, Antiphospholipid syndrome (APS), Autoimmune angioedema, Autoimmune aplastic anemia, Autoimmune dysautonomia, Autoimmune hepatitis, Autoimmune hyperlipidemia, Autoimmune immunodeficiency, Autoimmune inner ear disease (AIED), Autoimmune myocarditis, Autoimmune oophoritis, Autoimmune pancreatitis, Autoimmune retinopathy, Autoimmune thrombocytopenic purpura (ATP), Autoimmune thyroid disease, Autoimmune urticaria, Axonal & neuronal neuropathies, Balo disease, Behcet's disease, Bullous pemphigoid, Cardiomyopathy, Castleman disease, Celiac disease, Chagas disease, Chronic fatigue syndrome, Chronic inflammatory demyelinating polyneuropathy (CIDP), Chronic recurrent multifocal ostomyelitis (CRMO), Churg-Strauss syndrome, Cicatricial pemphigoid/benign mucosal pemphigoid, Crohn's disease, Cogans syndrome, Cold agglutinin disease, Congenital heart block, Coxsackie myocarditis, CREST disease, Essential mixed cryoglobulinemia, Demyelinating neuropathies, Dermatitis herpetiformis, Dermatomyositis, Devic's disease (neuromyelitis optica), Discoid lupus, Dressler's syndrome, Endometriosis, Eosinophilic esophagitis, Eosinophilic fasciitis, Erythema nodosum, Experimental allergic encephalomyelitis, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis (GPA) (formerly called Wegener's Granulomatosis), Graves' disease, Guillain-Barre syndrome, Hashimoto's encephalitis, Hashimoto's thyroiditis, Hemolytic anemia, Henoch-Schonlein purpura, Herpes gestationis, Hypogammaglobulinemia, Idiopathic thrombocytopenic purpura (ITP), IgA nephropathy, IgG4-related sclerosing disease, Immunoregulatory lipoproteins, Inclusion body myositis, Interstitial cystitis, Juvenile arthritis, Juvenile diabetes (Type 1 diabetes), Juvenile myositis, Kawasaki syndrome, Lambert-Eaton syndrome, Leukocytoclastic vasculitis, Lichen planus, Lichen sclerosus, Ligneous conjunctivitis, Linear IgA disease (LAD), Lupus (SLE), Lyme disease, chronic, Meniere's disease, Microscopic polyangiitis, Mixed connective tissue disease (MCTD), Mooren's ulcer, Mucha-Habermann disease, Multiple sclerosis, Myasthenia gravis, Myositis, Narcolepsy, Neuromyelitis optica (Devic's), Neutropenia, Ocular cicatricial pemphigoid, Optic neuritis, Palindromic rheumatism, PANDAS (Pediatric Autoimmune Neuropsychiatric Disorders Associated with Streptococcus), Paraneoplastic cerebellar degeneration, Paroxysmal nocturnal hemoglobinuria (PNH), Parry Romberg syndrome, Parsonnage-Turner syndrome, Pars planitis (peripheral uveitis), Pemphigus, Peripheral neuropathy, Perivenous encephalomyelitis, Pernicious anemia, POEMS syndrome, Polyarteritis nodosa, Type I, II, & III autoimmune polyglandular syndromes, Polymyalgia rheumatica, Polymyositis, Postmyocardial infarction syndrome, Postpericardiotomy syndrome, Progesterone dermatitis, Primary biliary cirrhosis, Primary sclerosing cholangitis, Psoriasis, Psoriatic arthritis, Idiopathic pulmonary fibrosis, Pyoderma gangrenosum, Pure red cell aplasia, Raynauds phenomenon, Reactive Arthritis, Reflex sympathetic dystrophy, Reiter's syndrome, Relapsing polychondritis, Restless legs syndrome, Retroperitoneal fibrosis, Rheumatic fever, Rheumatoid arthritis, Sarcoidosis, Schmidt syndrome, Scleritis, Scleroderma, Sjogren's syndrome, Sperm & testicular autoimmunity, Stiff person syndrome, Subacute bacterial endocarditis (SBE), Susac's syndrome, Sympathetic ophthalmia, Takayasu's arteritis, Temporal arteritis/Giant cell arteritis, Thrombocytopenic purpura (TTP), Tolosa-Hunt syndrome, Transverse myelitis, Type 1 diabetes, Ulcerative colitis, Undifferentiated connective tissue disease (UCTD), Uveitis, Vasculitis, Vesiculobullous dermatosis, Vitiligo, Wegener's granulomatosis (now termed Granulomatosis with Polyangiitis (GPA), and the like.

In some instances, immune-related disorders may also include an activated immune system, e.g., as present in a subject fighting an infection or other immune stimulating condition.

Kits

Also provided are kits for practicing one or more of the above-described methods. The subject kits may vary greatly. Reagents and devices included in the subject kits include those mentioned above with respect to the methods of detecting per cell PD-L1 expression in a bladder-associated sample, and also method detecting whether an immune cell that expresses PD-L1 above a predetermined threshold is present in a bladder-associated sample, detecting a PD-L1-aneuploid-to-PD-L1-epithelial ratio of a sample, and/or identifying whether a malignant bladder-associated neoplasia is present in a subject.

These would include, for example, specific binding members for PD-L1, including, for example, a labeled binding member specific for PD-L1, an antibody specific for PD-L1, and the like. Subject kits may further include one or more sample preparation reagents including but not limited to, e.g., cell fixatives, cell permeabilizing reagents, cell labeling reagents, buffers, diluents, etc. The above components may be present in separate containers or one or more components may be combined into a single container, e.g., a glass or plastic vial. In some instances, kits of the instant disclosure may further include a sample preparation device such as e.g., a homogenizer.

Kits may further include sample obtainment devices. Useful sample obtainment devices may vary and may include but are not limited to e.g., bladder-associated sample collection containers, including those configured to collect liquid samples from a subject, such as urine, cytology effluent (e.g., bladder-irrigation fluid), etc. Useful bladder-associated sample collection containers for collecting urine or cytology effluent (e.g., bladder-irrigation fluid) include but are not limited to e.g., a urine sample collection cup, urinary drainage bags, and the like. In some instances, a urinary catheter may be a useful obtainment device that may be included in a subject kit.

Useful sample obtainment devices may also include those devices configured for the obtainment of solid and/or semi-solid samples, such as biopsies. Accordingly, useful sample obtainment devices may also include biopsy collection devices, such as but not limited to e.g., bladder biopsy collection devices, urinary tract biopsy collection devices, cystoscope components, and the like. In some instances, a kit of the present disclosure may include a cystoscope, or components thereof, that includes a bladder biopsy collection device configured to operate in conjunction with the cystoscope.

Further non-limiting examples of biopsy collection devices include but are not limited to e.g., needle biopsy devices, core biopsy devices, punch biopsy devices, surgical biopsy devices, vacuum assisted biopsy devices, etc.

In some instances, kits may further include one or more reagents and/or devices for cell dissociation including but not limited to e.g., enzymes, enzyme inhibitors, detergents, cell dissociation media or buffer, vortex devices, nutating devices, rocking devices, etc.

Subject kits may further include control reagents and samples including but not limited to, e.g., control cell samples (e.g., positive control cellular samples, negative control cellular samples, etc.), calibration reagents (e.g., fluorescent beads, pre-labeled cells, etc.), and the like.

In addition to the above components, the subject kits may further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address which may be used via the internet to access the information at a removed site. Any convenient means may be present in the kits.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference. Reagents, cloning vectors, and kits for genetic manipulation referred to in this disclosure are available from commercial vendors such as BioRad, Stratagene, Invitrogen, Sigma-Aldrich, and ClonTech.

### Example 1

Patient urine samples (or samples of urine in ThinPrep or LiquiPrep solutions) were centrifuged to form cell pellets. Samples were aspirated without disturbing cell pellets and the cell pellets were resuspended in PBS. Resuspended cells were mixed with 1X IncelIMAX (IncellDx, Inc.; San Carlos, Calif.) fixative at 1:1 ratio. Samples were incubated in fixative for 1 hour and, following incubation, samples were centrifuged and the supernatant was discarded.

Labeling master mixes were prepared containing DAPI, anti-CD45 antibody, anti-PD-L1 antibody and anti-pan cytokeratin antibody. Aliquots of labeling master mix were added to each sample and labeling was performed for about 30 minutes after which time additional BSA-containing buffer was added to the samples. Samples were centrifuged, supernatant was aspirated, and samples were washed an additional time with BSA-containing buffer.

Prepared sample was loaded into a CytoFLEX (Beckman Coulter; Indianapolis, Ind.) instrument that had been recently undergone standard quality control procedures and been calibrated with 8 Peak Beads and configured with appropriate compensation. Data for 50,000 events was recorded (over about 5-10 min. collection time) under medium flow rate. Collected data parameters included: percent (%) single nucleated cells; percent (%) CD45+ cells; percent (%) Pan Ck(+) CD45(−) cells; percent (%) PD-L1(+) CD45(−) cells; percent (%) PD-L1(+) CD45(+) cells; percent (%) Pan Ck(+) PD-L1 (−) cells; percent (%) Pan Ck(+) PD-L1 (+) cells; and DNA content (DNA index).

Results

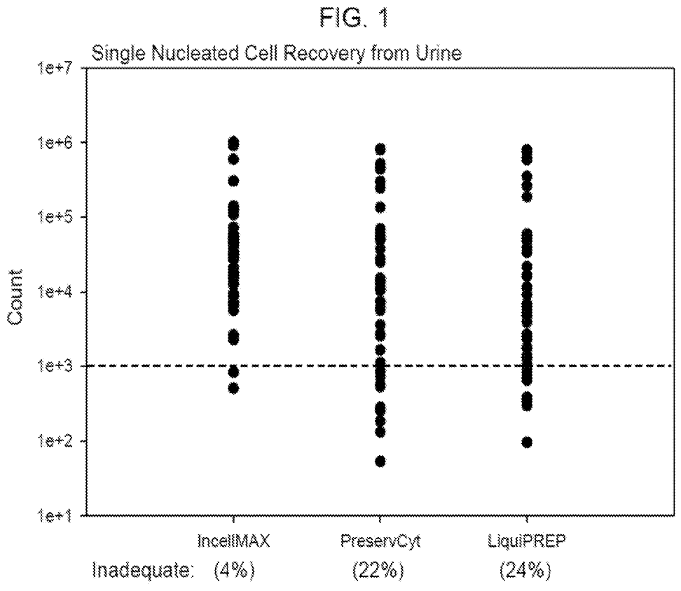

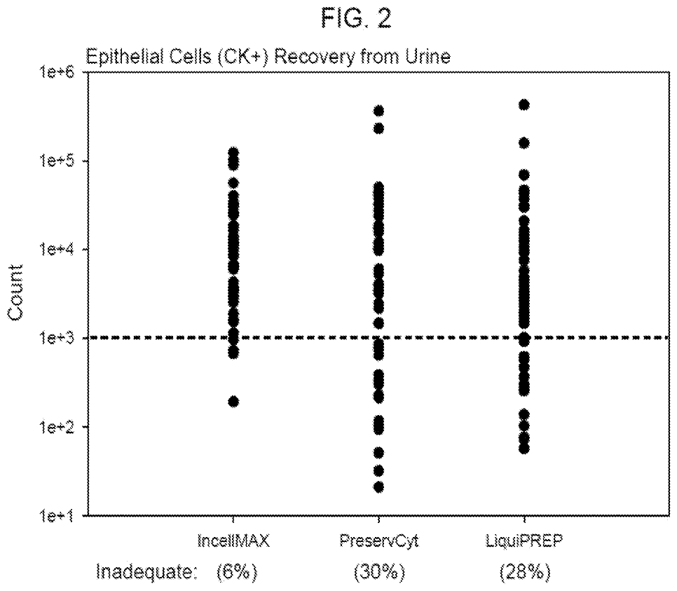

Sample adequacy and cell recovery was initially assessed and the performance of InceMax (IncelIDx, San Carlos, Calif.), LiquiPREP (LGM International Inc.; Melbourne, Fla.), and PerservCyt (Hologic Inc.; Marlborough, Mass.) was compared. IncelIMAX demonstrated better cell recovery, including for single nucleated cells (FIG. 1) and epithelial cells (CK+) (FIG. 2), from urine samples and improves sample adequacy compared to other reagents (dotted lines indicate the threshold for sample adequacy). However, these results also demonstrate that various cell preparation reagents are capable of generating samples that are adequate for analysis by the methods described herein.

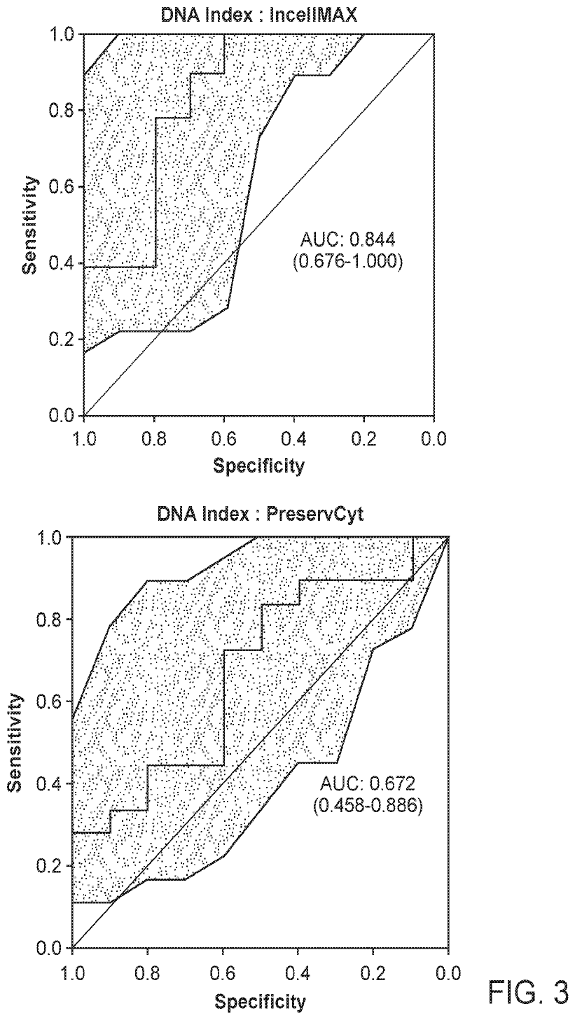

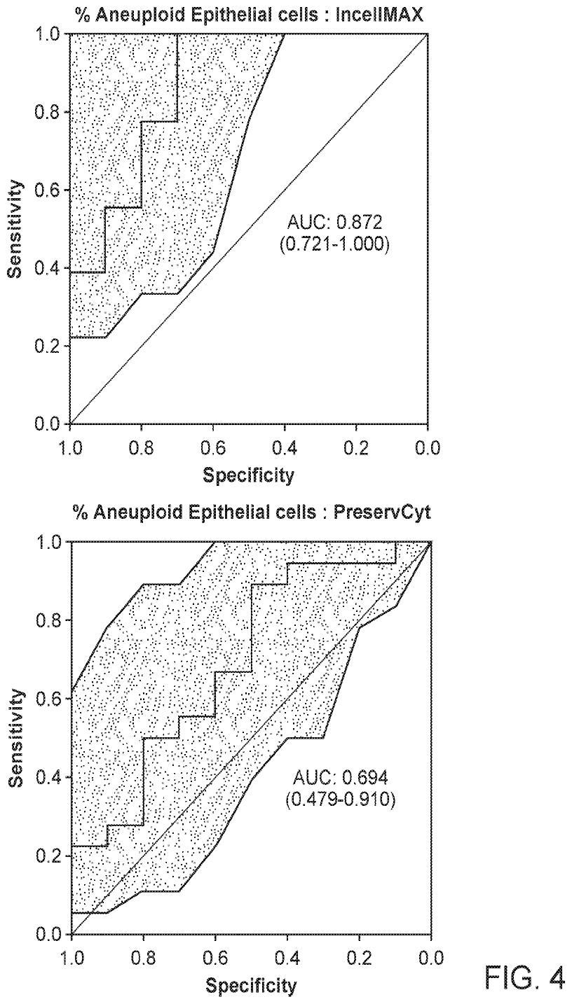

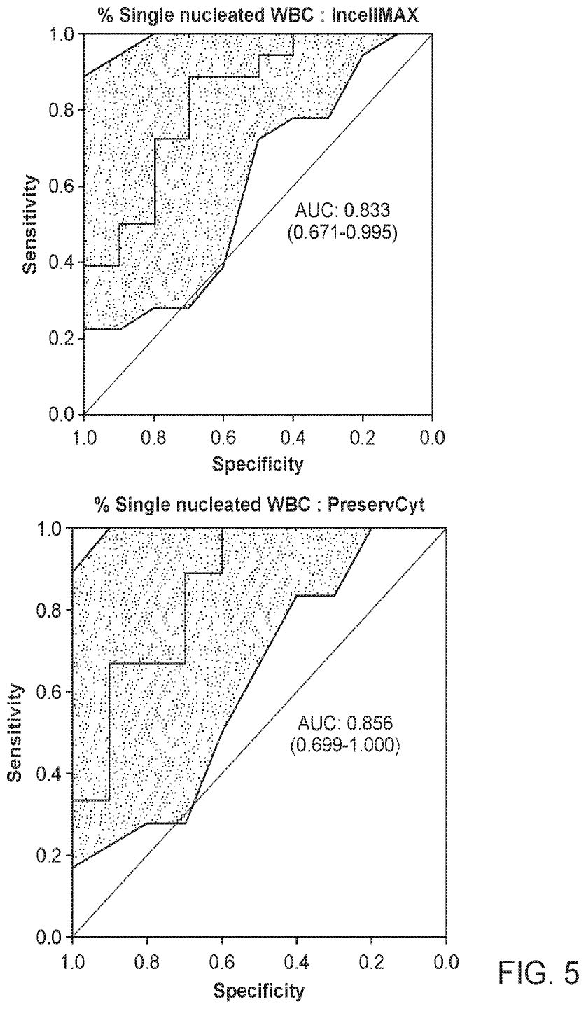

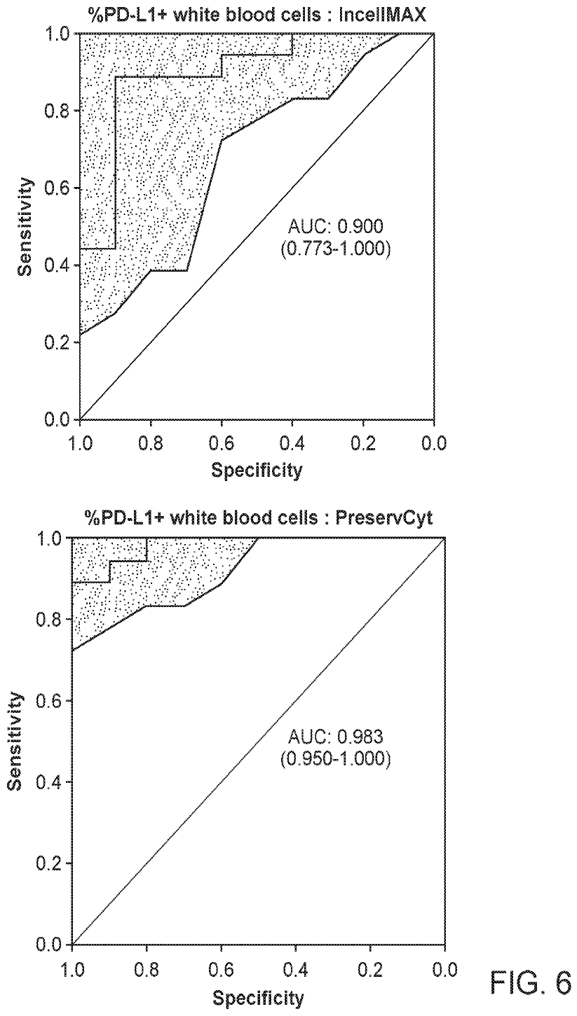

Individual ROC curves for significant parameters, including DNA index (FIG. 3), percent (%) aneuploid epithelial cells (FIG. 4), percent (%) single nucleated white blood cells (WBC) (FIG. 5), and percent (%) PD-L1(+) WBC (FIG. 6), are provided for samples prepared using IncelIMAX and PerservCyt. These curves demonstrate the predictive power of each individual classifier and, particularly, demonstrate the high predictive power of percent (%) PD-L1(+) WBC as a classifier for bladder cancer samples.

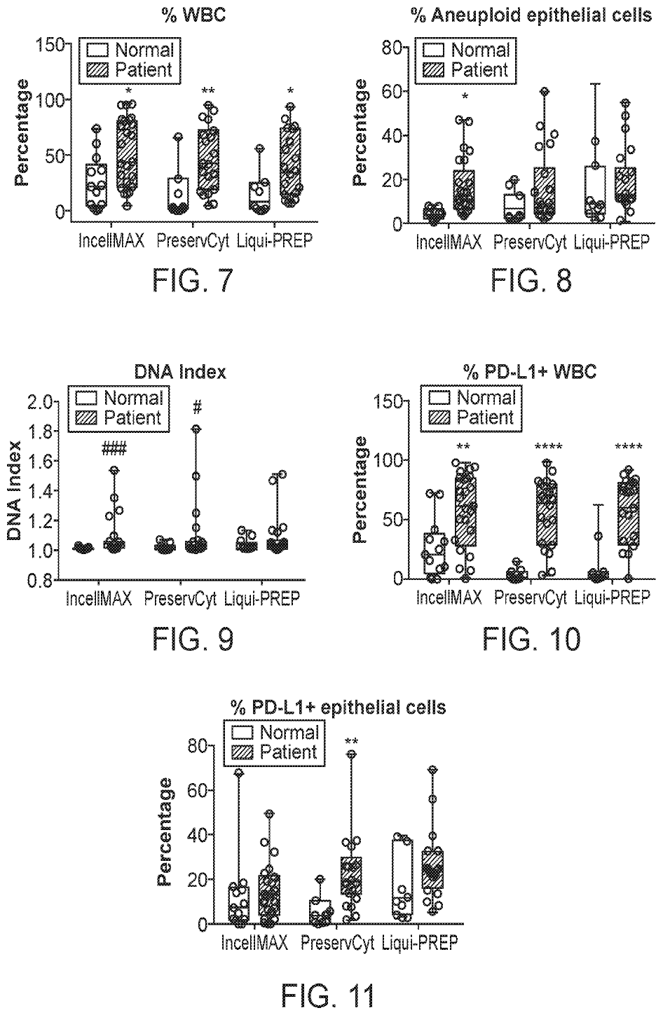

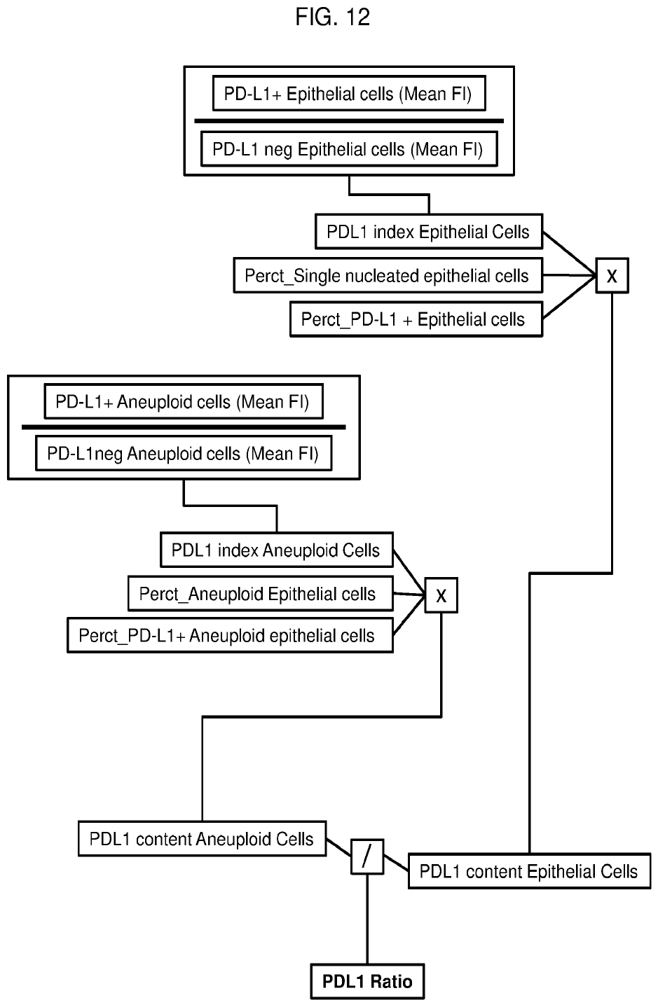

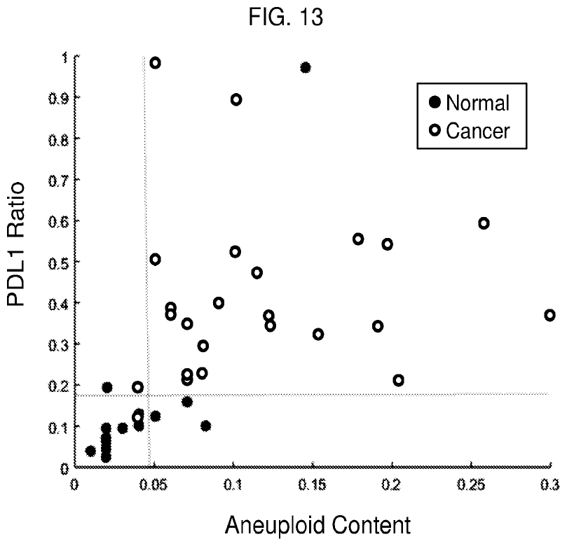

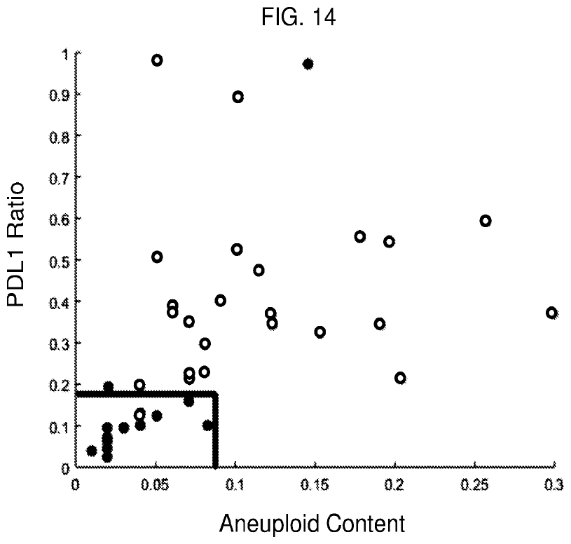

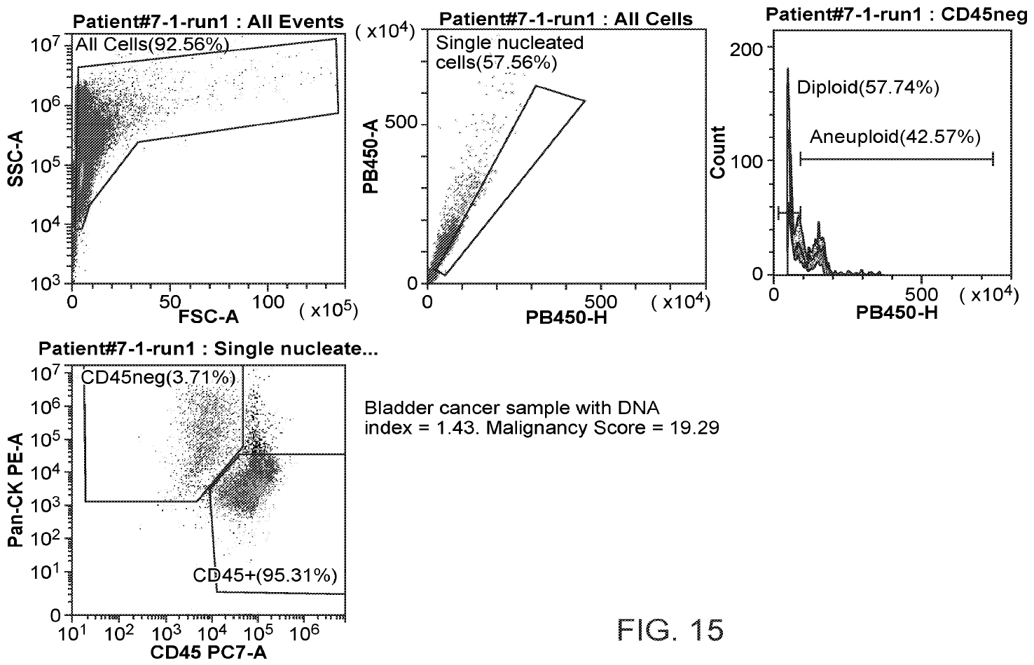

Box-and-whisker plots summarizing the statistical findings for: percent single nucleated white blood cells (% WBC) (FIG. 7), percent (%) aneuploid epithelial cells (FIG. 8), DNA index (FIG. 9), percent (%) PD-L1(+) WBC (FIG. 10), percent (%) PD-L1(+) epithelial cells (FIG. 11), are also provided. Box-and-whisker plots represent values within the interquartile range (boxes) and the minimum to maximum (whiskers). The line within the box shows the median. Each data point is shown as an open dot. The “Normal” group included n=11-13. The “Patient” group included n=22-25. Samples with fewer than 1000 single nucleated cells were excluded from the analysis. Statistical thresholds as determined by 2-way ANOVA, Bonferroni's post-test are depicted in the figures as: *P<0.05, **P<0.005, ****P<0.0001 vs. normal group. Statistical thresholds as determined by Mann-Whitney test are depicted in the figures as: # P<0.05, ### P<0.0005 vs. normal group.

Analysis that included the use of percent (%) PD-L1(+) WBC as a classifier for bladder cancer (“patient”) samples versus “normal” samples demonstrated 97% sensitivity and 92% specificity. The statistical associations between parameters and disease status are provided in Table 2 below. These analyses demonstrate a consistent statistical association between PD-L1 positivity and disease status across all fixation methods.

TABLE 2

| Variable | IncellMAX | LiquiPREP | PreservCyt |
|---|---|---|---|
| % Aneuploid Epithelial cells | 0.000739 | 0.207851 | 0.098664 |
| DNA Index | 0.002081 | 0.132742 | 0.145850 |
| % PD-L1 + Aneuploid | 0.723860 | 0.008354 | 0.001398 |
| epithelial cells | | | |
| % PD-L1 + Epithelial cells | 0.493672 | 0.064177 | 0.003038 |
| % PD-L1 + white blood cells | 0.000225 | 0.000021 | 0.000001 |
| % Single nucleated WBC | 0.003038 | 0.002520 | 0.001398 |

Overall, the statistical analyses performed demonstrate that the analyzed parameters may be effectively employed in methods of identifying subjects having bladder cancer, and/or metastatic bladder cancer, and, in particular, PD-L1 positivity (alone or in combination with other parameters) may be used to identify such subjects with high sensitivity and specificity. In addition, while IncelIMAX was shown to most effectively recover cells from urine samples for analysis, other sample preparation reagents were capable of generating adequate samples and PD-L1 positivity correlated with disease status regardless of the sample preparation reagent employed.

Materials

The following stock reagents were employed in this example: 1×DPBS, 10% BSA, DPBS+2% BSA, Anti PD-L1 (AF647), Anti CD45 (PC7), Anti Pan Cytokeratin (PE), Cell Cycle Dye (1 mg bottle), Cell Cycle Dye (1 mg/mL in water), OncoTect iO Control Cells, Lyophilized OncoTect iO Control Cells Buffer, and 8-Peak Beads. OncoTect iO Control cells and buffer were warmed to RT and cells were reconstituted with buffer. PBS+2% BSA solution was prepared from a 1:5 dilution of 10% BSA in DPBS. A 1 mg/mL solution of DAPI in DPBS was also prepared.

### Example 2

Patient urine samples were prepared, labeled and analyzed essentially as described above in Example 1. The following features were measured:
Perct_Single nucleated epithelial cells (corresponding to the percent of single cells in the sample that are nucleated and labeled with a labeled binding member specific for epithelial cells),
Perct_PD-L1+Epithelial cells (corresponding to the percent of cells labeled with a labeled binding member specific for epithelial cells that are also labeled with a labeled binding member specific for PD-L1),
Perct_Single nucleated WBC (corresponding to the percent of single cells in the sample that are nucleated and labeled with a labeled binding member specific for white blood cells),
Perct_PD-L1+white blood cells (corresponding to the percent of cells labeled with a labeled binding member specific for WBCs that are also labeled with a labeled binding member specific for PD-L1),
Perct_Aneuploid Epithelial cells (corresponding to the percent of cells labeled with a labeled binding member specific for epithelial cells that are also aneuploid),
Perct_PD-L1+Aneuploid epithelial cells (corresponding to the percent of aneuploid cells that are also labeled with a labeled binding member specific for PD-L1),
DNA Index (corresponding to the ratio of the amount of DNA labeling reagent fluorescence per cell of the sample to the amount expected of a normal diploid cell),
PD-L1+Epithelial cells (Mean FI) (corresponding to the mean fluorescence attributable to cells labeled with a labeled binding member specific for epithelial cells and the labeled binding member specific for PD-L1),
PD-L1 neg Epithelial cells (Mean FI) (corresponding to the mean fluorescence attributable to cells labeled with a labeled binding member specific for epithelial cells and not with the labeled binding member specific for PD-L1),
PD-L1+Aneuploid cells (Mean FI) (corresponding to the mean fluorescence attributable to aneuploid cells labeled with the labeled binding member specific for PD-L1),
PD-L1neg Aneuploid cells (Mean FI) (corresponding to the mean fluorescence attributable to aneuploid cells not with the labeled binding member specific for PD-L1),
PD-L1+WBC cells (Mean FI) (corresponding to the mean fluorescence attributable to cells labeled with a labeled binding member specific for WBCs and labeled with the labeled binding member specific for PD-L1), and
PD-L1neg WBC cells (Mean FI) (corresponding to the mean fluorescence attributable to cells labeled with a labeled binding member specific for WBCs and not with the labeled binding member specific for PD-L1)

From the above measured features new derived index features were developed that included the following:

PDL1 index Epithelial Cells=(PD-L1+Epithelial cells (Mean FI)/PD-L1 neg Epithelial cells (Mean FI)),

PDL1 index Aneuploid Cells=(PD-L1+Aneuploid cells (Mean FI)/PD-L1neg Aneuploid cells (Mean FI)), and

PDL1 index WBC Cells=(PD-L1+WBC cells (Mean FI)/PD-L1neg WBC cells (Mean FI)).

From the above measured and derived index features new derived contents features were developed that included the following:

PDL1 content Epithelial Cells=(Perct_Single nucleated epithelial cells)*(Perct_PD-L1+Epithelial cells)*(PDL1 index Epithelial Cells),

PDL1 content WBC Cells=(Perct_Single nucleated WBC)*(Perct_PD-L1+white blood cells)*(PDL1 index WBC Cells),

PDL1 content Aneuploid Cells=(Perct_Aneuploid Epithelial cells)*(Perct_PD-L1+Aneuploid epithelial cells)*(PDL1 index Aneuploid Cells),

Aneuploid Content=(DNA Index*Perct_Aneuploid Epithelial cells),

Aneuploid Content PDL1 positive cells=(DNA Index)*(Perct_Aneuploid Epithelial cells)*(Perct_PD-L1+Aneuploid epithelial cells), and

Aneuploid PDL1 content=(Perct_Aneuploid Epithelial cells)*(Perct_PD-L1+Aneuploid epithelial cells)*(PDL1 index Aneuploid Cells).

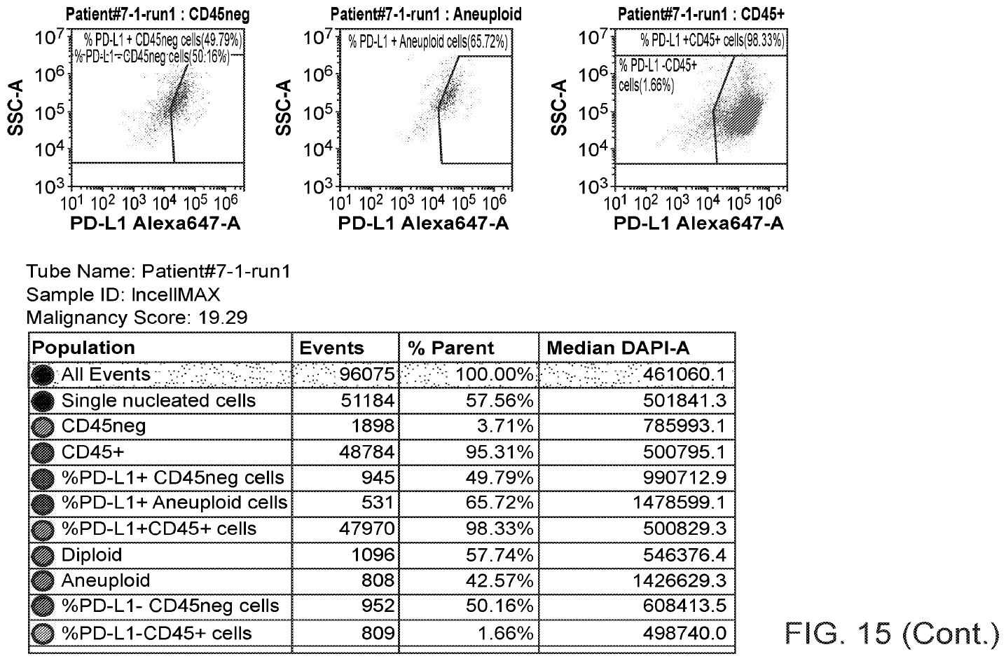

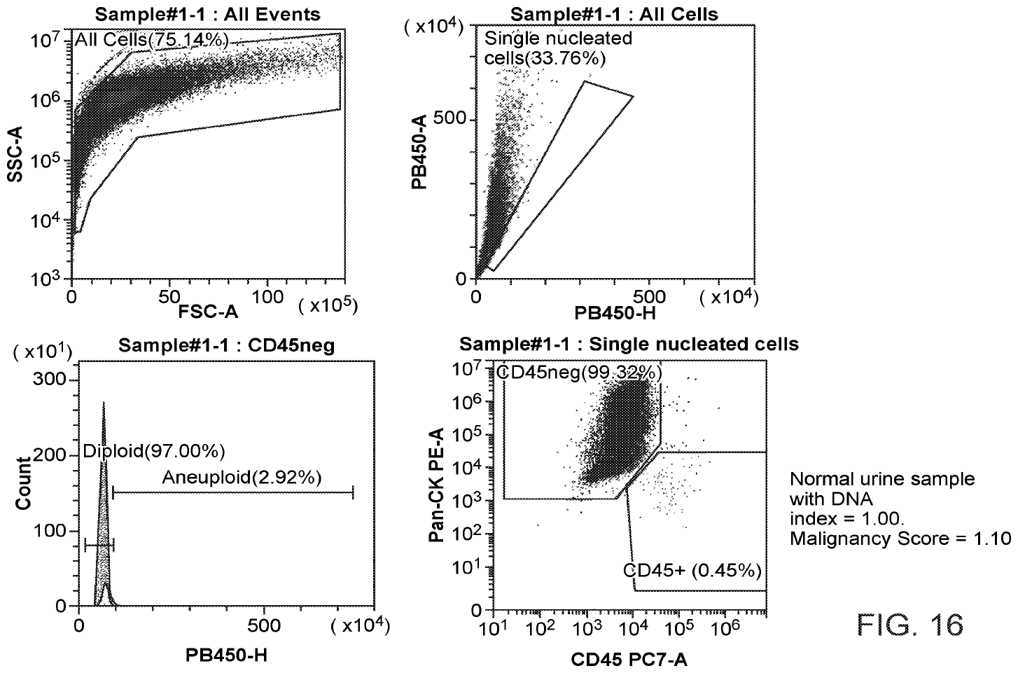

Next a PD-L1 Ratio parameter, corresponding to the ratio of PDL1 content Aneuploid Cells to PDL1 content Epithelial Cells (i.e., a PD-L1-aneuploid-to-PD-L1-epithelial ratio), for bladder-associated samples was created employing the measured and derived features described above. As depicted in the flowchart of FIG. 12, the PD-L1 Ratio=[(Perct_Aneuploid Epithelial cells)*(Perct_PD-L1+Aneuploid epithelial cells)*(PD-L1+Aneuploid cells (Mean FI)/(PD-L1neg Aneuploid cells (Mean FI)]/[(Perct_Single nucleated epithelial cells)*(Perct_PD-L1+Epithelial cells)*(PD-L1+Epithelial cells (Mean FI)/PD-L1 neg Epithelial cells (Mean FI))]. A plot of normal (i.e., unaffected) patient samples (closed circles) and cancerous (i.e., affected) patient samples according to PD-L1 Ratio and Aneuploid Content is provided in FIG. 13, showing clustering of normal samples as compared to the distribution of cancer samples.

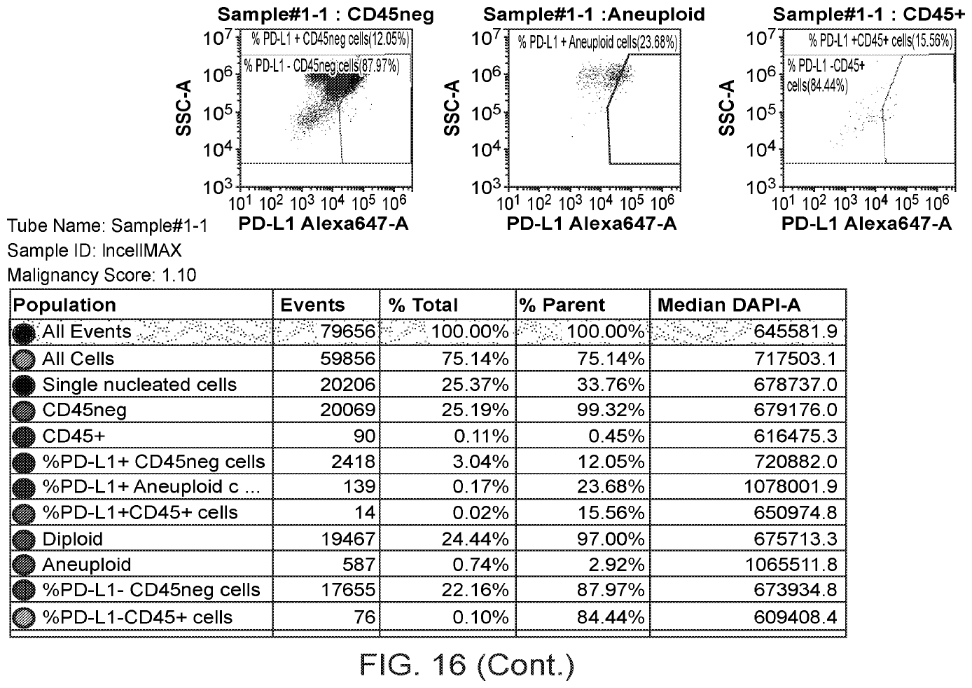

Using dual thresholds of either less than 0.18 for PD-L1 Ratio and 0.09 for Aneuploid Content (as shown in FIG. 14) produced only 1 of 50 false negatives and 2 of 15 false positives, corresponding to a sensitivity of 98% and specificity of 87%. This level of sensitivity and specificity may be achieved employing the PD-L1 Ratio alone. However, in some instances, employing the rule that a test sample is positive if PD-L1 ratio is greater than 0.18 OR Aneuploid Content is greater than 0.09 may increase robustness of the assay. This example demonstrates the derivation of a PD-L1 Ratio feature and the usefulness of employing a detected PD-L1 Ratio, with or without an Aneuploid Content feature, to detect the presence or absence of malignant bladder-associated neoplasia with high sensitivity and specificity.

### Example 3-Adaptive Genetic Algorithms Combined with High Sensitivity Single Cell-Based Technology Derived Urine-Based Score is Potentially Capable of Differentiating Between High Grade and Low Grade Transitional Cell Carcinoma of the Bladder

Objective:

Above we show that adaptive genetic algorithms (AGA), in combination with single-cell flow cytometry technology, can be used to develop a noninvasive urine-based score to detect bladder cancer with high sensitivity. Our aim in this analysis was to investigate if that same score can differentiate between high grade (HG) and low grade (LG) transitional cell carcinoma of the bladder (BC).

Materials and Methods:

We collected urine samples from cystoscopy confirmed HG and LG superficial bladder cancer patients and healthy donors in an optimized urine collection media. We then examined these samples using an assay developed from AGA in combination with single-cell flow cytometry technology.

Results:

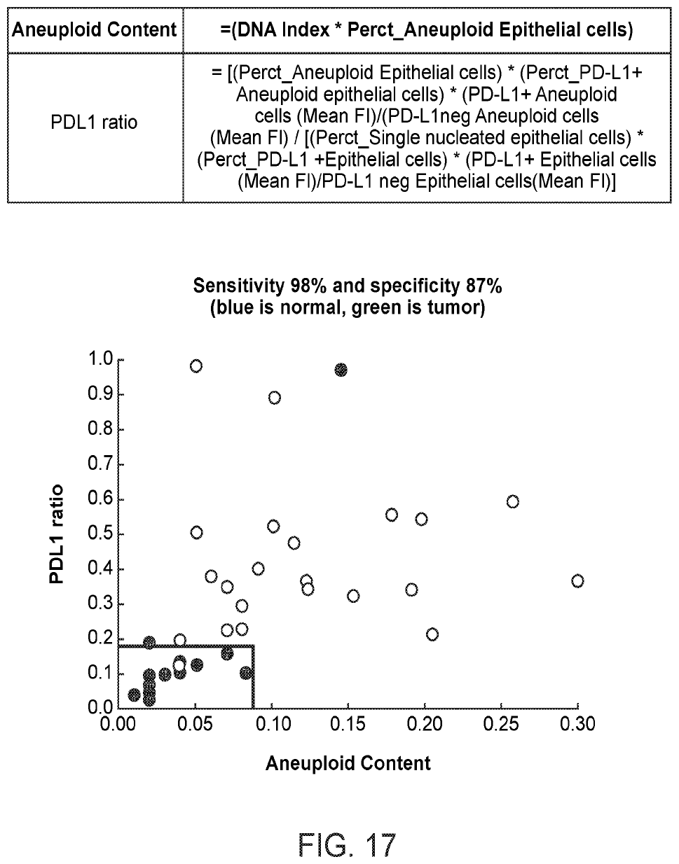

We examined 50 BC and 15 healthy donor urine samples. Patients were majorly White (59.2%), males (61.2%), and had HG BC (66.7%). AGA derived score of 1.1 differentiated between BCa and healthy patients with high precision (AUC 0.92). The median score was 2.8 for LG BC and 6 for HG BC. Mann-Whitney Rank Sum Test indicated that the difference between the median score of HG and LG BC was significant at P value=0.003. The score performed well independent of patients' sex or smoking history. FIGS. 15 to 17 provide further details.

### Conclusions

Using single-cell technology and machine learning, we developed a new urine-based score that can differentiate between HG and LG bladder cancer.

Notwithstanding the appended claims, the disclosure is also defined by the following clauses:

1. A method comprising:
contacting a bladder-associated sample from a subject with a labeled binding member specific for programmed-death ligand 1 (PD-L1) to generate a labeled cell suspension; and
cytometrically assaying the labeled cell suspension to quantify per cell PD-L1 expression.
   2. The method according to Clause 1, wherein the bladder-associated sample is prepared from a urine or cytology effluent sample.
   3. The method according to Clause 1 or 2, wherein the method further comprises contacting the bladder-associated sample with a DNA labelling reagent, at least one labeled binding member specific for immune cells, at least one labeled binding member specific for epithelial cells, or a combination thereof to generate the labeled cell suspension.
   4. The method according to Clause 3, wherein the at least one labeled binding member specific for immune cells comprises an anti-CD45 antibody.
   5. The method according to Clause 3 or 4, wherein the at least one labeled binding member specific for epithelial cells comprises an anti-pan-cytokeratin antibody.
   6. The method according to any of Clauses 2 to 5, wherein the method comprises enriching the urine or cytology effluent sample for cells.
   7. The method according to Clause 6, wherein the enriching comprises centrifugation, filtration, or a combination thereof.
   8. The method according to any of the preceding clauses, wherein the cells of the labeled cell suspension sample are fixed.
   9. The method according to Clause 8, wherein the method further comprises fixing cells present in the bladder-associated sample before, during or after the contacting.
   10. The method according to Clause 9, wherein fixing the cells comprises contacting the cells of the bladder-associated sample with a mildly crosslinking agent.
   11. The method according to Clause 10, wherein the mildly crosslinking agent comprises a formaldehyde-based fixative.
   12. The method according to any of the preceding clauses, wherein the method comprises detecting whether an immune cell that expresses PD-L1 above a predetermined threshold is present in the bladder-associated sample.
   13. The method according to Clause 12, wherein the predetermined threshold is 100 or more PD-L1 molecules per cell.
   14. The method according to Clause 13, wherein the predetermined threshold is 500 or more PD-L1 molecules per cell.
   15. The method according to Clause 14, wherein the predetermined threshold is 1000 or more PD-L1 molecules per cell.
   16. The method according to any of the preceding clauses, wherein the bladder-associated sample is obtained from a subject having or suspected of having microscopic hematuria.
   17. The method according to any of the preceding clauses, wherein the method comprises detecting a PD-L1-aneuploid-to-PD-L1-epithelial ratio of a bladder-associated sample.
   18. The method according to Clause 17, wherein the PD-L1-aneuploid-to-PD-L1-epithelial ratio comprises the ratio of a PD-L1 content parameter for aneuploid cells of the bladder-associated sample to a PD-L1 content parameter for epithelial cells of the bladder-associated sample.
   19. The method according to Clause 18, wherein the PD-L1 content parameter for aneuploid cells comprises the product of: (i) an aneuploid-epithelial population size value, (ii) a PD-L1 positive aneuploid-epithelial population size value, and (iii) a PD-L1 content index for aneuploid cells.
   20. The method according to Clause 19, wherein the PD-L1 content index for aneuploid cells comprises the ratio of PD-L1 positive aneuploid cells to PD-L1 negative aneuploid cells.
   21. The method according to any of Clauses 18 to 20, wherein the PD-L1 content parameter for epithelial cells comprises the product of: (i) an epithelial population size value, (ii) a PD-L1 positive epithelial population size value, and (iii) a PD-L1 content index for epithelial cells.
   22. The method according to Clause 21, wherein PD-L1 content index for epithelial cells comprises the ratio of PD-L1 positive epithelial cells to PD-L1 negative epithelial cells.
   23. The method according to any of the preceding clauses, wherein the method further comprises determining an epithelial-aneuploid content value of the bladder-associated sample.
   24. The method according to Clause 23, wherein the epithelial-aneuploid content value comprises the product of a DNA index of the sample and the aneuploid-epithelial population size value of the sample.
   25. The method according to any of the preceding clauses, further comprising identifying whether a malignant bladder-associated neoplasia is present in the subject based on:
detecting whether an immune cell that expresses PD-L1 is present in the bladder-associated sample;
detecting the PD-L1-aneuploid-to-PD-L1-epithelial ratio; or
a combination thereof.
   26. The method according to Clause 25, wherein the identifying comprises identifying the presence of the malignant bladder-associated neoplasia when the PD-L1-aneuploid-to-PD-L1-epithelial ratio is above a threshold and identifying the absence of the malignant bladder-associated neoplasia when the PD-L1-aneuploid-to-PD-L1-epithelial ratio is below the threshold.
   27. The method according to Clause 26, wherein the threshold is 0.2 or less.
   28. The method according to any of Clauses 25 to 27, wherein the identifying is further based on detecting the epithelial-aneuploid content value.
   29. The method according to Clause 28, wherein the presence of the malignant bladder-associated neoplasia is identified when the PD-L1-aneuploid-to-PD-L1-epithelial ratio and the epithelial-aneuploid content value are both above predetermined threshold values and the absence of the malignant bladder-associated neoplasia is identified when the PD-L1-aneuploid-to-PD-L1-epithelial ratio and the epithelial-aneuploid content value are both below predetermined threshold values.
   30. The method according to Clause 29, wherein the predetermined threshold value for the PD-L1-aneuploid-to-PD-L1-epithelial ratio is 0.2 or less and the predetermined threshold value for the epithelial-aneuploid content value is 0.1 or less.
   31. The method according to any of Clauses 25 to 30, wherein the identifying is further based on a further cytometric parameter produced from quantifying:
the percentage of cells that are immune cells,
per cell DNA index,
epithelial cell aneuploidy, or
a combination thereof.
   32. The method according to Clause 31, wherein the further cytometric parameter comprises the percentage of cells that are immune cells and the threshold is an immune cell percentage of 5% or greater.
   33. The method according to Clause 31 or 32, wherein the further cytometric parameter comprises per cell DNA index and the threshold is a per cell DNA index of 1.1 or greater.
   34. The method according to any of Clauses 31 to 33, wherein the further cytometric parameter comprises epithelial cell aneuploidy and the threshold is a percentage of aneuploid epithelial cells of 5% or greater.
   35. A method of treating a subject for a malignant bladder-associated neoplasia, the method comprising:
administering an anti-PD-1/PD-L1 immunotherapy to a subject comprising a malignant bladder-associated neoplasia identified according to the method of any of Clauses 25 to 34.
   36. The method according to Clause 35, wherein the subject has been previously treated with chemotherapy.
   37. The method according to Clauses 35 or 36, wherein the subject has been previously treated with radiation therapy.
   38. The method according to any of Clauses 35 to 37, wherein the subject has been previously treated with immunotherapy.
   39. The method according to any of Clauses 35 to 38, wherein the subject has an immune-related disorder or is at increased risk of developing an immune-related disorder.
   40. The method according to any of Clauses 35 to 39, wherein the anti-PD-1/PD-L1 immunotherapy comprises administering to the subject one or more anti-PD-1/PD-L1 therapeutic antagonists selected from the group consisting of: OPDIVO® (nivolumab), KEYTRUDA® (pembrolizumab), Tecentriq™ (atezolizumab), durvalumab (MEDI4736), avelumab (MSB0010718C), BMS-936559 (MDX-1105), CA-170, BMS-202, BMS-8, BMS-37 and BMS-242.
   41. The method according to any of Clauses 35 to 40, wherein the method further comprises monitoring the malignant bladder-associated neoplasia for anti-PD-1/PD-L1 immunotherapy responsiveness during the therapy and continuing the therapy only when the neoplasia is identified as anti-PD-1/PD-L1 immunotherapy responsive.
   42. A method of detecting whether an immune cell that expresses programmed-death ligand 1 (PD-L1) above a predetermined threshold is present in a bladder-associated sample, the method comprising:
contacting the bladder-associated sample with a labeled binding member specific for PD-L1 to generate a labeled cell suspension; and
cytometrically assaying the labeled cell suspension to quantify per cell PD-L1 expression to detect whether an immune cell that expresses PD-L1 above a predetermined threshold is present in the bladder-associated sample.
   43. The method according to Clause 42, wherein the method comprises contacting the bladder-associated sample with at least one labeled binding member specific for immune cells.
   44. The method according to Clause 43, wherein the at least one labeled binding member specific for immune cells comprises an anti-CD45 antibody.
   45. The method according to any of Clauses 42 to 44, wherein the bladder-associated sample is obtained from a subject having or suspected of having microscopic hematuria.
   46. The method according to any of Clauses 42 to 45, wherein the bladder-associated sample is a liquid sample.
   47. The method according to Clause 46, wherein the liquid sample is urine.
   48. The method according to Clause 47, wherein the urine is collected via micturition or catheterization.
   49. The method according to any of Clauses 42 to 46, wherein the liquid sample is cytology effluent.
   50. The method according to any of Clauses 46 to 49, wherein the method comprises enriching the liquid sample for cells.
   51. The method according to Clause 50, wherein the enriching comprises centrifugation, filtration, or a combination thereof.
   52. The method according to any of Clauses 42 to 45, wherein the bladder-associated sample is a liquid cellular sample prepared from a solid tissue sample.
   53. The method according to Clause 52, wherein the solid tissue sample is a bladder biopsy.
   54. The method according to Clauses 52 or 53, wherein the method comprises dissociating the solid tissue sample to generate the liquid cellular sample.
   55. The method according to any of Clauses 42 to 54, wherein the cells of the labeled cell suspension sample are fixed.
   56. The method according to Clause 55, wherein the method further comprises fixing cells present in the bladder-associated sample before, during or after the contacting.
   57. The method according to Clause 56, wherein fixing the cells comprises contacting the cells of the bladder-associated sample with a mildly crosslinking agent.
   58. The method according to Clause 57, wherein the mildly crosslinking agent comprises a formaldehyde-based fixative.
   59. The method according to any of Clauses 42 to 58, wherein the predetermined threshold is 100 or more PD-L1 molecules per cell.
   60. The method according to Clause 59, wherein the predetermined threshold is 500 or more PD-L1 molecules per cell.
   61. The method according to Clause 60, wherein the predetermined threshold is 1000 or more PD-L1 molecules.
   62. A method of identifying whether a malignant bladder-associated neoplasia is present in a subject, the method comprising:
detecting whether an immune cell that expresses programmed-death ligand 1 (PD-L1) is present in a bladder-associated sample from the subject according to any of Clauses 42 to 61;
further cytometrically assaying the labeled cell suspension to quantify:
   the percentage of cells that are immune cells,
   per cell DNA index,
   epithelial cell aneuploidy, or
   a combination thereof, to produce a further cytometric parameter; and identifying:
   the presence of the malignant bladder-associated neoplasia when the immune cell that expresses PD-L1 is present and the further cytometric parameter is above a threshold, or
   the absence of the malignant bladder-associated neoplasia when the immune cell that expresses PD-L1 is absent and/or the further cytometric parameter is below the threshold.
      63. The method according to Clause 62, wherein the method further comprises contacting the bladder-associated sample with a DNA labeling reagent, at least one labeled binding member specific for epithelial cells, at least one labeled binding member specific for immune cells, or a combination thereof.
      64. The method according to Clause 63, wherein the at least one labeled binding member specific for immune cells comprises an anti-CD45 antibody.
      65. The method according to Clauses 63 or 64, wherein the at least one labeled binding member specific for epithelial cells comprises an anti-pan-cytokeratin antibody. 66. The method according to any of Clauses 62 to 65, wherein the further cytometric parameter comprises the percentage of cells that are immune cells and the threshold is an immune cell percentage of 5% or greater.
      67. The method according to any of Clauses 62 to 66, wherein the further cytometric parameter comprises per cell DNA index and the threshold is a per cell DNA index of 1.1 or greater.
      68. The method according to any of Clauses 62 to 67, wherein the further cytometric parameter comprises epithelial cell aneuploidy and the threshold is a percentage of aneuploid epithelial cells of 5% or greater.
      69. A method of treating a subject for a malignant bladder-associated neoplasia, the method comprising:
administering an anti-PD-1/PD-L1 immunotherapy to a subject comprising a malignant bladder-associated neoplasia identified according to the method of any of Clauses 62 to 68.
   70. The method according to Clause 69, wherein the subject has been previously treated with chemotherapy.
   71. The method according to Clauses 69 or 70, wherein the subject has been previously treated with radiation therapy.
   72. The method according to any of Clauses 69 to 71, wherein the subject has been previously treated with immunotherapy.
   73. The method according to any of Clauses 69 to 72, wherein the subject has an immune-related disorder or is at increased risk of developing an immune-related disorder.
   74. The method according to any of Clauses 69 to 73, wherein the anti-PD-1/PD-L1 immunotherapy comprises administering to the subject one or more anti-PD-1/PD-L1 therapeutic antagonists selected from the group consisting of: OPDIVO® (nivolumab), KEYTRUDA® (pembrolizumab), Tecentriq™ (atezolizumab), durvalumab (MEDI4736), avelumab (MSB0010718C), BMS-936559 (MDX-1105), CA-170, BMS-202, BMS-8, BMS-37 and BMS-242.
   75. The method according to any of Clauses 69 to 74, wherein the method further comprises monitoring the malignant bladder-associated neoplasia for anti-PD-1/PD-L1 immunotherapy responsiveness during the therapy and continuing the therapy only when the neoplasia is identified as anti-PD-1/PD-L1 immunotherapy responsive.
   76. A kit comprising:
a bladder-associated sample collection container; and
a labeled binding member specific for PD-L1.
   77. The kit according to Clause 76, wherein the bladder-associated sample collection container comprises a urine sample collection cup or urinary drainage bag.
   78. The kit according to Clauses 76 or 77, wherein the kit further comprises a urinary catheter.
   79. The kit according to any of Clause 76 to 78, wherein the kit further comprises a bladder biopsy collection device.
   80. The kit according to any of Clause 76 to 79, wherein the kit comprises a cystoscope.
   81. The kit according to Clause 80, wherein the cystoscope comprises the bladder biopsy collection device.
   82. The kit according to any of Clauses 76 to 81, wherein the kit further comprises a fixation reagent.
   83. The kit according to Clause 82, wherein the fixation reagent is a mildly crosslinking agent.
   84. The kit according to Clause 83, wherein the mildly crosslinking agent comprises a formaldehyde-based fixative.
   85. The kit according to any of Clauses 76 to 84, further comprising a DNA labeling reagent.
   86. The kit according to any of Clauses 76 to 85, further comprising at least one labeled binding member specific for immune cells.
   87. The kit according to Clause 86, wherein the at least one labeled binding member specific for immune cells comprises a labeled binding member specific for lymphocyte marker CD45.
   88. The kit according to any of Clauses 76 to 87, further comprising at least one labeled binding member specific for epithelial cells.
   89. The kit according to Clause 88, wherein the at least one labeled binding member specific for epithelial cells comprises a labeled binding member specific for cytokeratin.
   90. A labeled bladder-associated sample, the sample comprising an immune cell expressing PD-L1 and a labeled binding member specific for PD-L1 noncovalently bound to the immune cell.
   91. The sample according to Clause 90, further comprising at least one labeled binding member specific for immune cells noncovalently bound to the immune cell.
   92. The sample according to Clause 91, wherein the at least one labeled binding member specific for immune cells comprises an anti-CD45 antibody.
93. The sample according to any of Clauses 90 to 92, wherein the bladder-associated sample is obtained from a subject having or suspected of having microscopic hematuria.
   94. The sample according to any of Clauses 90 to 93, wherein the bladder-associated sample is a liquid sample.
   95. The sample according to Clause 94, wherein the liquid sample is urine.
   96. The sample according to Clause 94, wherein the liquid sample is cytology effluent.
   97. The sample according to Clause 94, wherein the liquid cellular sample is a dissociated solid tissue sample.
   98. The sample according to Clause 97, wherein the dissociated solid tissue sample is a dissociated bladder biopsy.
   99. The sample according to any of Clauses 90 to 98, wherein the sample is enriched for cells.
   100. The sample according to any of Clauses 90 to 99, wherein the cells of the sample are fixed using a fixation reagent.
   101. The sample according to Clause 100, wherein the fixation reagent is a mildly crosslinking agent.
   102. The sample according to Clause 101, wherein the mildly crosslinking agent comprises a formaldehyde-based fixative.
   103. The sample according to any of Clauses 90 to 102, wherein the sample further comprises a DNA labeling reagent, at least one labeled binding member specific for epithelial cells, or a combination thereof.
   104. The sample according to Clause 103, wherein the at least one labeled binding member specific for epithelial cells comprises an anti-pan-cytokeratin antibody.
   105. A cytometric device comprising the labeled bladder-associated sample according to any of Clauses 90 to 104.
   106. The cytometric device according to Clause 105, wherein the cytometric device is a flow cytometer.
   107. The cytometric device according to Clause 105, wherein the cytometric device is a cell cytometer.
   108. A method of identifying whether a malignant bladder-associated neoplasia is present in a subject, the method comprising:
detecting a PD-L1-aneuploid-to-PD-L1-epithelial ratio of a bladder-associated sample from the subject, the detecting comprising:
   contacting the bladder-associated sample with a labeled binding member specific for PD-L1 and a DNA labelling reagent to generate a labeled cell suspension; and
   cytometrically assaying the labeled cell suspension to quantify per cell PD-L1 expression and DNA content to generate the PD-L1-aneuploid-to-PD-L1-epithelial ratio; and
identifying:
   the presence of the malignant bladder-associated neoplasia when the ratio is above a predetermined threshold, or
   the absence of the malignant bladder-associated neoplasia when the ratio is below the predetermined threshold.
      109. The method according to Clause 108, wherein the PD-L1-aneuploid-to-PD-L1-epithelial ratio comprises the ratio of a PD-L1 content parameter for aneuploid cells of the bladder-associated sample to a PD-L1 content parameter for epithelial cells of the bladder-associated sample.
      110. The method according to Clause 109, wherein the PD-L1 content parameter for aneuploid cells comprises the product of: (i) an aneuploid-epithelial population size value, (ii) a PD-L1 positive aneuploid-epithelial population size value, and (iii) a PD-L1 content index for aneuploid cells.
      111. The method according to Clause 110, wherein the PD-L1 content index for aneuploid cells comprises the ratio of PD-L1 positive aneuploid cells to PD-L1 negative aneuploid cells.
      112. The method according to any of Clauses 109 to 111, wherein the PD-L1 content parameter for epithelial cells comprises the product of: (i) an epithelial population size value, (ii) a PD-L1 positive epithelial population size value, and (iii) a PD-L1 content index for epithelial cells.
      113. The method according to Clause 112, wherein PD-L1 content index for epithelial cells comprises the ratio of PD-L1 positive epithelial cells to PD-L1 negative epithelial cells.
      114. The method according to any of Clauses 108 to 113, wherein the predetermined threshold is 0.2 or less.
      115. The method according to any of Clauses 108 to 114, wherein the method further comprises determining an epithelial-aneuploid content value of the bladder-associated sample.
      116. The method according to Clause 115, wherein the presence of the malignant bladder-associated neoplasia is identified when the PD-L1-aneuploid-to-PD-L1-epithelial ratio and the epithelial-aneuploid content value are both above predetermined threshold values and the absence of the malignant bladder-associated neoplasia is identified when the PD-L1-aneuploid-to-PD-L1-epithelial ratio and the epithelial-aneuploid content value are both below predetermined threshold values.
      117. The method according to Clauses 115 or 116, wherein the epithelial-aneuploid content value comprises the product of a DNA index of the sample and the aneuploid-epithelial population size value of the sample.
      118. The method according to Clause 117, wherein the predetermined threshold value for the PD-L1-aneuploid-to-PD-L1-epithelial ratio is 0.2 or less and the predetermined threshold value for the epithelial-aneuploid content value is 0.1 or less.
      119. The method according to any of Clauses 108 to 118, wherein the method further comprises contacting the bladder-associated sample with at least one labeled binding member specific for immune cells.
      120. The method according to Clause 119, wherein the method further comprises detecting whether an immune cell that expresses PD-L1 is present in a bladder-associated sample.
      121. A method of treating a subject for a malignant bladder-associated neoplasia, the method comprising:
administering an anti-PD-1/PD-L1 immunotherapy to a subject comprising a malignant bladder-associated neoplasia identified according to the method of any of Clauses 108 to 120.
   122. The method according to Clause 121, wherein the subject has been previously treated with chemotherapy.
   123. The method according to Clauses 121 or 122, wherein the subject has been previously treated with radiation therapy.
   124. The method according to any of Clauses 121 to 123, wherein the subject has been previously treated with immunotherapy.
   125. The method according to any of Clauses 121 to 124, wherein the subject has an immune-related disorder or is at increased risk of developing an immune-related disorder.
   126. The method according to any of Clauses 121 to 125, wherein the anti-PD-1/PD-L1 immunotherapy comprises administering to the subject one or more anti-PD-1/PD-L1 therapeutic antagonists selected from the group consisting of: OPDIVO® (nivolumab), KEYTRUDA® (pembrolizumab), Tecentriq™ (atezolizumab), durvalumab (MEDI4736), avelumab (MSB0010718C), BMS-936559 (MDX-1105), CA-170, BMS-202, BMS-8, BMS-37 and BMS-242.
   127. The method according to any of Clauses 121 to 126, wherein the method further comprises monitoring the malignant bladder-associated neoplasia for anti-PD-1/PD-L1 immunotherapy responsiveness during the therapy and continuing the therapy only when the neoplasia is identified as anti-PD-1/PD-L1 immunotherapy responsive.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112(6) is not invoked.

What is claimed is:

1. A method comprising:
contacting a bladder-associated sample from a subject with a labeled binding member specific for programmed-death ligand 1 (PD-L1) to generate a labeled cell suspension; and
cytometrically assaying the labeled cell suspension to quantify per cell PD-L1 expression.

2. The method according to claim 1, wherein the bladder-associated sample is prepared from a urine or cytology effluent sample.

3. The method according to claim 1 or 2, wherein the method further comprises contacting the bladder-associated sample with a DNA labelling reagent, at least one labeled binding member specific for immune cells, at least one labeled binding member specific for epithelial cells, or a combination thereof to generate the labeled cell suspension.

4. The method according to any of the preceding claims, wherein the cells of the labeled cell suspension sample are fixed.

5. The method according to claim 4, wherein the method further comprises fixing cells present in the bladder-associated sample before, during or after the contacting.

6. The method according to any of the preceding claims, wherein the method comprises detecting whether an immune cell that expresses PD-L1 above a predetermined threshold is present in the bladder-associated sample.

7. The method according to any of the preceding claims, wherein the method comprises detecting a PD-L1-aneuploid-to-PD-L1-epithelial ratio of a bladder-associated sample.

8. The method according to any of the preceding claims, further comprising identifying whether a malignant bladder-associated neoplasia is present in the subject based on:
detecting whether an immune cell that expresses PD-L1 is present in the bladder-associated sample;
detecting the PD-L1-aneuploid-to-PD-L1-epithelial ratio; or
a combination thereof.

9. A method of treating a subject for a malignant bladder-associated neoplasia, the method comprising:
administering an anti-PD-1/PD-L1 immunotherapy to a subject comprising a malignant bladder-associated neoplasia identified according to the method of any of claims 1 to 8.

10. A method of detecting whether an immune cell that expresses programmed-death ligand 1 (PD-L1) above a predetermined threshold is present in a bladder-associated sample, the method comprising:
contacting the bladder-associated sample with a labeled binding member specific for PD-L1 to generate a labeled cell suspension; and
cytometrically assaying the labeled cell suspension to quantify per cell PD-L1 expression to detect whether an immune cell that expresses PD-L1 above a predetermined threshold is present in the bladder-associated sample.

11. A method of identifying whether a malignant bladder-associated neoplasia is present in a subject, the method comprising:
detecting whether an immune cell that expresses programmed-death ligand 1 (PD-L1) is present in a bladder-associated sample from the subject according to 10;
further cytometrically assaying the labeled cell suspension to quantify:
the percentage of cells that are immune cells,
per cell DNA index,
epithelial cell aneuploidy, or
a combination thereof, to produce a further cytometric parameter; and
identifying:
the presence of the malignant bladder-associated neoplasia when the immune cell that expresses PD-L1 is present and the further cytometric parameter is above a threshold, or
the absence of the malignant bladder-associated neoplasia when the immune cell that expresses PD-L1 is absent and/or the further cytometric parameter is below the threshold.

12. A method of treating a subject for a malignant bladder-associated neoplasia, the method comprising:
administering an anti-PD-1/PD-L1 immunotherapy to a subject comprising a malignant bladder-associated neoplasia identified according to the method of claim 11.

13. A kit comprising:
a bladder-associated sample collection container; and
a labeled binding member specific for PD-L1.

14. A labeled bladder-associated sample, the sample comprising an immune cell expressing PD-L1 and a labeled binding member specific for PD-L1 noncovalently bound to the immune cell.
